(19) United States
(12) Patent Application Publication
(10) Pub. No.: US 2015/0133512 A1
(43) Pub. Date: May 14, 2015

(54) N-ARYLTRIAZOLE COMPOUNDS AS LPAR ANTAGONISTS

(71) Applicant: Hoffmann-La Roche Inc,, Nutley, NJ (US)

(72) Inventors: Stephen Deems Grabriel, Morristown, NJ (US); Matthew Michael Hamilton, Hackettstown, NJ (US); Yimin Qian, Plainsboro, NJ (US); Achyutharao Sidduri, Newark, NJ (US)

(21) Appl. No.: 14/403,261

(22) PCT Filed: Jun. 17, 2013
(86) PCT No.: PCT/EP2013/062463

§ 371 Date: Nov. 24, 2014

Related U.S. Application Data
(60) Provisional application No. 61/661,953, filed on Jun. 20, 2012.

(51) Int. Cl. C07D 249/06

(52) U.S. Cl. CPC C07D 249/06
USPC 514/359; 548/255

(57) ABSTRACT

Provided herein are compounds of the formula (I): as well as pharmaceutically acceptable salts thereof, wherein the substituents are as those disclosed in the specification. These compounds, and the pharmaceutical compositions containing them, are useful for the treatment of inflammatory diseases and disorders such as, for example, pulmonary fibrosis.

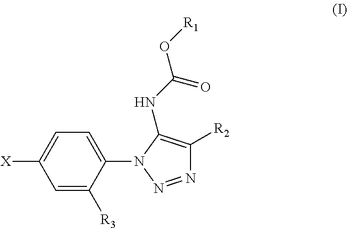

The present invention relates to organic compounds useful for therapy and/or prophylaxis in a mammal of an inflammatory disease or disorder, and in particular to N-aryltriazole compounds, their manufacture, pharmaceutical compositions containing them and their use as lysophosphatidic acid (LPA) antagonists.

LPA is a family of bioactive phosphate lipids which function like a growth factor mediator by interacting with LPA receptors, a family of G-protein-coupled receptors (GPCRs). The lipid family has long chain saturated (such as C18:0 or C16:0) or unsaturated (C18:1 or C20:4) carbon chains attached to the glycerol through an ester linkage. In biological systems, LPA is produced by multi-step enzymatic pathways through the de-esterification of membrane phospholipids. Enzymes that contribute to LPA synthesis include lysophospholipase D (lysoPLD), autotaxin (ATX), phospholipase A1 (PLA1), phospholipase A2 (PLA2) and acylglycerol kinase (AGK) (British J. of Pharmacology 2012, 165, 829-844).

There are at least six LPA receptors identified (LPAR1-6). LPA signaling exerts a broad range of biological responses on many different cell types, which can lead to cell growth, cell proliferation, cell migration and cell contraction. Up regulation of the LPA pathway has been linked to multiple diseases, including cancer, allergic airway inflammation, and fibrosis of the kidney, lung and liver. Therefore, targeting LPA receptors or LPA metabolic enzymes could provide new approaches towards the treatment of medically important diseases that include neuropsychiatric disorders, neuropathic pain, infertility, cardiovascular disease, inflammation, fibrosis, and cancer (Annu. Rev. Pharmacol. Toxicol. 2010, 50, 157-186; J. Biochem. 2011, 150, 223-232).

Fibrosis is the result of an uncontrolled tissue healing process leading to excessive accumulation of extracellular matrix (ECM). Recently it was reported that the LPA1 receptor was over expressed in idiopathic pulmonary fibrosis (IPF) patients. Mice with LPA1 receptor knockout were protected from bleomycin-induced lung fibrosis (Nature Medicine 2008, 14, 45-54).

Thus, antagonizing LPA1 receptor may be useful for the treatment of fibrosis, such as renal fibrosis, pulmonary fibrosis, arterial fibrosis and systemic sclerosis.

In an embodiment of the present invention, provided are compounds of general formula (I):

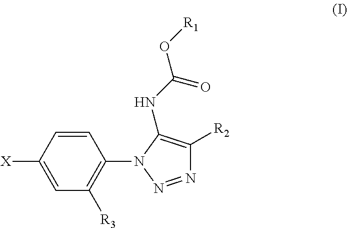

wherein:
R₁ is lower alkyl or indanyl, said lower alkyl being unsubstituted or substituted with cycloalkyl, unsubstituted phenyl or phenyl substituted with halogen or —CF₃;
R₂ is hydrogen or lower alkyl;
R₃ is hydrogen, fluorine or —OCH₃;
X is cycloalkyl acetic acid or

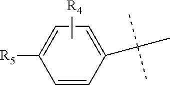

R₄ is hydrogen or halogen;
R₅ is hydrogen, cyano, tetrazole-cyclopropyl, methanesulfonylaminocarbonyl-cyclopropyl or

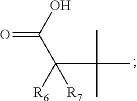

R₆ and R₇ are, independently of each other, hydrogen or lower alkyl; or
R₆ and R₇, together with the carbon to which they are attached, form a cycloalkyl group,
or a pharmaceutically acceptable salt thereof.

In another embodiment of the present invention, provided are compounds of general formula (I), (Ia), (Ib) or (Ic):

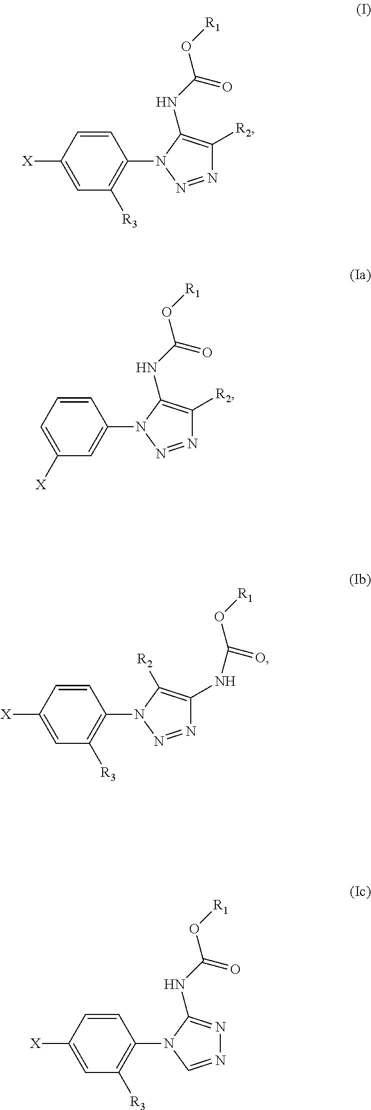

wherein:
R₁ is lower alkyl or indanyl, said lower alkyl being unsubstituted or substituted with cycloalkyl, unsubstituted phenyl or phenyl substituted with halogen or —CF₃;
R₂ is hydrogen or lower alkyl;
R₃ is hydrogen, fluorine or —OCH₃;
is cycloalkyl acetic acid or

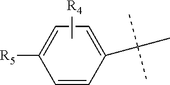

R₄ is hydrogen or halogen;
R₅ is hydrogen, cyano, tetrazole-cyclopropyl, methanesulfonylaminocarbonyl-cyclopropyl or

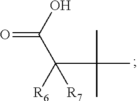

R₆ and R₇ are, independently of each other, hydrogen or lower alkyl; or
R₆ and R₇, together with the carbon to which they are attached, form a cycloalkyl group,
or a pharmaceutically acceptable salt thereof.

In another embodiment of the present invention, provided are compounds of general formula (I), (Ia), (Ib) or (Ic):

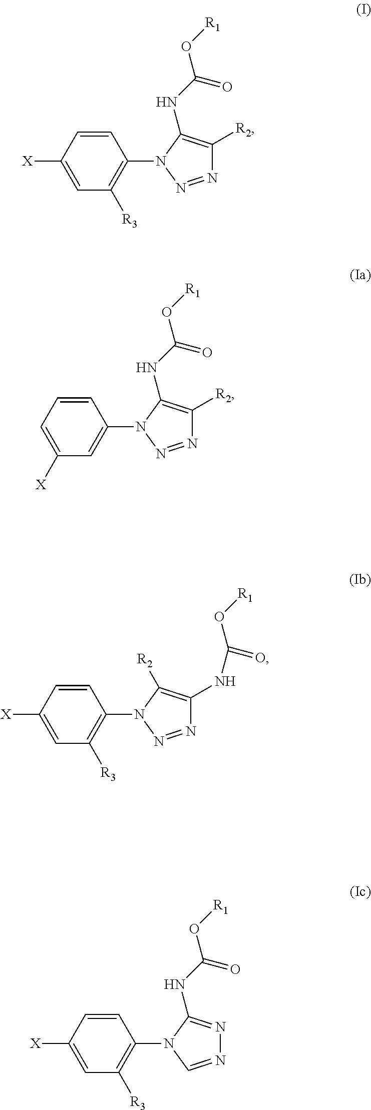

wherein:
R₁ is lower alkyl or indanyl, said lower alkyl being unsubstituted or substituted with cycloalkyl, unsubstituted phenyl or phenyl substituted with halogen or —CF₃;
R₂ is hydrogen or lower alkyl;
R₃ is hydrogen, fluorine or —OCH₃;
X is cycloalkyl acetic acid or

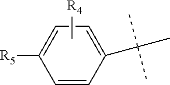

R₄ is hydrogen or halogen;
R₅ is hydrogen, cyano, tetrazole-cyclopropyl, methanesulfonylaminocarbonyl-cyclopropyl or

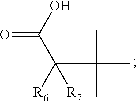

R₆ and R₇ are, independently of each other, hydrogen, lower alkyl or lower alkenyl; or
R₆ and R₇, together with the carbon to which they are attached, form a cycloalkyl group,
or a pharmaceutically acceptable salt thereof.

In a further embodiment of the invention, provided is a pharmaceutical composition comprising a therapeutically effective amount of a compound according to formula (I) and a therapeutically inert carrier.

In a still further embodiment of the invention, provided is a method for the treatment or prophylaxis of pulmonary fibrosis, which method comprises the step of administering a therapeutically effective amount of a compound according to formula (I) to a patient in need thereof.

All documents cited to or relied upon below are expressly incorporated herein by reference.

Unless otherwise indicated, the following specific As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “lower alkyl”, alone or in combination with other groups, refers to a branched or straight-chain alkyl radical of one to nine carbon atoms, preferably one to six carbon atoms, more preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, adamantyl and the like. In a preferred embodiment, the “cycloalkyl” moieties can optionally be substituted with one, two, three or four substituents, with the understanding that said substituents are not, in turn, substituted further. Each substituent can independently be, alkyl, alkoxy, halogen, amino, hydroxyl or oxygen (O═) unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate, with the understanding that said substituents are not, in turn, substituted further.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthalene, 1,2-dihydronaphthalene, indanyl, 1H-indenyl and the like.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. Examples of such groups include, but are not limited to, pyridine, thiazole and pyranyl.

The alkyl, lower alkyl, aryl and heteroaryl groups described above may be substituted independently with one, two, or three substituents, with the understanding that said substituents are not, in turn, substituted further. Substituents may include, for example, halogen, lower alkyl, —CF₃, —SO₂CH₃, alkoxy, —C(O)CH₃, —OH, —SCH₃ and —CH₂CH₂OH.

As used herein, the term “alkoxy” means alkyl-O—; and “alkoyl” means alkyl-CO—. Alkoxy substituent groups or alkoxy-containing substituent groups may be substituted by, for example, one or more alkyl groups, with the understanding that said substituents are not, in turn, substituted further.

As used herein, the term “halogen” means a fluorine, chlorine, bromine or iodine radical, preferably a fluorine, chlorine or bromine radical, and more preferably a fluorine or chlorine radical.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminum salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

In one embodiment of the present invention, provided is a compound of formula (I) wherein R₁ is dimethylpropyl, butyl or isopropyl.

In another embodiment of the present invention, provided is a compound of formula (I) wherein R₁ is lower alkyl substituted with cycloalkyl, unsubstituted phenyl or phenyl substituted with halogen or —CF₃.

In another embodiment of the present invention, provided is a compound of formula (I) wherein R₁ is —CH(CH₃)-phenyl, —CH(CH₃)-fluorophenyl, —CH(CH₃)-trifluoromethylphenyl, ethyl-cyclopropyl or ethyl-cyclobutyl.

In another embodiment of the present invention, provided is a compound of formula (I) wherein R₂ is lower alkyl.

In another embodiment of the present invention, provided is a compound of formula (I) wherein R₂ is methyl.

In another embodiment of the present invention, provided is a compound of formula (I) wherein R₃ is hydrogen.

In another embodiment of the present invention, provided is a compound of formula (I) wherein X is cyclohexyl acetic acid.

In another embodiment of the present invention, provided is a compound of formula (I) wherein X is

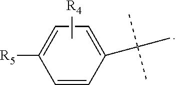

In another embodiment of the present invention, provided is a compound of formula (I) wherein R₄ is hydrogen or fluorine.

In another embodiment of the present invention, provided is a compound of formula (I) wherein R₅ is hydrogen, cyano, tetrazole-cyclopropyl or methanesulfonylaminocarbonyl-cyclopropyl.

In another embodiment of the present invention, provided is a compound of formula (I) wherein R₅ is

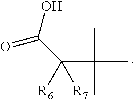

In another embodiment of the present invention, provided is a compound of formula (I) wherein R₆ and R₇ are, independently of each other, hydrogen or methyl.

In another embodiment of the present invention, provided is a compound of formula (I) wherein R₆ and R₇, together with the carbon to which they are attached, form a cyclopropyl group.

In another embodiment of the present invention, provided are compounds of general formula (I) wherein R₁ is lower alkyl or indanyl, said lower alkyl being unsubstituted or substituted with cycloalkyl or unsubstituted phenyl; R₂ is hydrogen or lower alkyl; R₃ is hydrogen, fluorine or —OCH₃; X is cycloalkyl acetic acid or

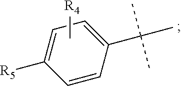

; wherein R₄ is hydrogen or halogen and R₅ is hydrogen, cyano, tetrazole-cyclopropyl, methanesulfonylaminocarbonyl-cyclopropyl or

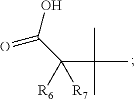

wherein R₆ and R₇ are, independently of each other, hydrogen or lower alkyl; or R₆ and R₇, together with the carbon to which they are attached, form a cycloalkyl group, or a pharmaceutically acceptable salt thereof.

In another embodiment of the present invention, provided are compounds of general formula (I) wherein R₁ is lower alkyl being substituted with unsubstituted phenyl; R₂ is hydrogen or lower alkyl; R₃ is hydrogen, fluorine or —OCH₃; X is cycloalkyl acetic acid or

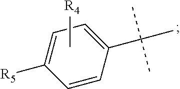

wherein R₄ is hydrogen or halogen and R₅ is hydrogen, cyano, tetrazole cyclopropyl, methanesulfonylaminocarbonyl-cyclopropyl or

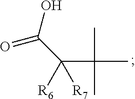

wherein R₆ and R₇ are, independently of each other, hydrogen or lower alkyl; or R₆ and R₇, together with the carbon to which they are attached, form a cycloalkyl group, or a pharmaceutically acceptable salt thereof.

In another embodiment of the present invention, provided are compounds of general formula (I) wherein R₁ is lower alkyl or indanyl, said lower alkyl being unsubstituted or substituted with cycloalkyl, unsubstituted phenyl or phenyl substituted with halogen or —CF₃; R₂ is ethyl; R₃ is hydrogen, fluorine or —OCH₃; X is cycloalkyl acetic acid or

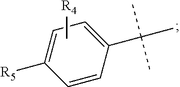

wherein R₄ is hydrogen or halogen and R₅ is hydrogen, cyano, tetrazole-cyclopropyl, methanesulfonylaminocarbonyl-cyclopropyl or

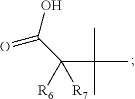

wherein R₆ and R₇ are, independently of each other, hydrogen or lower alkyl; or R₆ and R₇, together with the carbon to which they are attached, form a cycloalkyl group, or a pharmaceutically acceptable salt thereof.

In another embodiment of the present invention, provided are compounds of general formula (I) wherein R₁ is lower alkyl or indanyl, said lower alkyl being unsubstituted or substituted with cycloalkyl, unsubstituted phenyl or phenyl substituted with halogen or —CF₃; R₂ is hydrogen or lower alkyl; R₃ is hydrogen, fluorine or —OCH₃; X is cycloalkyl acetic acid or

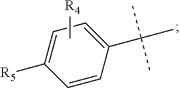

wherein R₄ is hydrogen or halogen and R₅ is

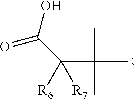

wherein R₆ and R₇, together with the carbon to which they are attached, form a cycloalkyl group, or a pharmaceutically acceptable salt thereof.

In another embodiment of the present invention, provided are compounds of general formula (I) wherein R₁ is lower alkyl being substituted with unsubstituted phenyl; R₂ is hydrogen or lower alkyl; R₃ is hydrogen, fluorine or —OCH₃; X is cycloalkyl acetic acid or

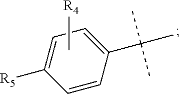

wherein R₄ is hydrogen or halogen and R₅ is

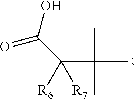

wherein R₆ and R₇, together with the carbon to which they are attached, form a cycloalkyl group, or a pharmaceutically acceptable salt thereof.

In another embodiment of the present invention, provided are compounds of general formula (I) wherein R₁ is lower alkyl or indanyl, said lower alkyl being unsubstituted or substituted with cycloalkyl, unsubstituted phenyl or phenyl substituted with halogen or —CF₃; R₂ is hydrogen or lower alkyl; R₃ is hydrogen, fluorine or —OCH₃; X is cycloalkyl acetic acid or

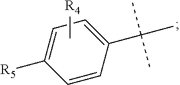

wherein R₄ is hydrogen or halogen and R₅ is methanesulfonylaminocarbonyl-cyclopropyl, or a pharmaceutically acceptable salt thereof.

In another embodiment of the present invention, provided are compounds of general formula (Ia) wherein R₁ is lower alkyl being substituted with unsubstituted phenyl; R₂ is lower alkyl; X is

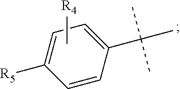

wherein R₄ is hydrogen and R₅ is

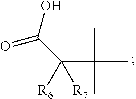

R₆ and R₇ are hydrogen or R₆ and R₇, together with the carbon to which they are attached, form a cycloalkyl group, or a pharmaceutically acceptable salt thereof.

In another embodiment of the present invention, provided are compounds of general formula (Ib) wherein R₁ is lower alkyl being substituted with unsubstituted phenyl; R₂ is lower alkyl; R₃ is hydrogen; X is

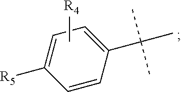

wherein R₄ is hydrogen and R₅ is

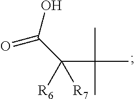

wherein R₆ and R₇ are hydrogen or R₆ and R₇, together with the carbon to which they are attached, form a cycloalkyl group, or a pharmaceutically acceptable salt thereof.

In another embodiment of the present invention, provided are compounds of general formula (Ic) wherein R₁ is lower alkyl being substituted with unsubstituted phenyl; R₃ is hydrogen; X is

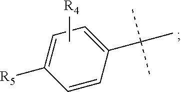

wherein R₄ is hydrogen and R₅ is

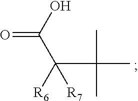

wherein R₆ and R₇, together with the carbon to which they are attached, form a cycloalkyl group, or a pharmaceutically acceptable salt thereof.

Particular compounds of formula (I) include the following:
1-{4′-[4-Methyl-5-((R)-1-phenyl-ethoxycarbonylamino)-[1,2,3]triazol-1-yl]-biphenyl-4-yl}-cyclopropanecarboxylic acid;
{4′-[4-Methyl-5-((R)-1-phenyl-ethoxycarbonylamino)-[1,2,3]triazol-1-yl]-biphenyl-4-yl}-acetic acid;
1-{4′-[5-Methyl-4-((R)-1-phenyl-ethoxycarbonylamino)-[1,2,3]triazol-1-yl]-biphenyl-4-yl}-cyclopropanecarboxylic acid;
{4′-[5-Methyl-4-((R)-1-phenyl-ethoxycarbonylamino)-[1,2,3]triazol-1-yl]-biphenyl-4-yl}-acetic acid;
1-(4′-{5-[(R)-1-(2-Fluoro-phenyl)-ethoxycarbonylamino]-4-methyl-[1,2,3]triazol-1-yl}-biphenyl-4-yl)-cyclopropanecarboxylic acid;
1-(4′-{4-Methyl-5-[(R)-1-(2-trifluoromethyl-phenyl)-ethoxycarbonylamino]-[1,2,3]triazol-1-yl}-biphenyl-4-yl)-cyclopropanecarboxylic acid;
1-(4′-{4-Methyl-5-[(R)-1-(3-trifluoromethyl-phenyl)-ethoxycarbonylamino]-[1,2,3]triazol-1-yl}-biphenyl-4-yl)-cyclopropanecarboxylic acid;
1-{4′-[5-((R)-Indan-1-yloxycarbonylamino)-4-methyl-[1,2,3]triazol-1-yl]-biphenyl-4-yl}-cyclopropanecarboxylic acid;
1-{4′-[5-((R)-1,2-Dimethyl-propoxycarbonylamino)-4-methyl-[1,2,3]triazol-1-yl]-biphenyl-4-yl}-cyclopropanecarboxylic acid;
1-{4′-[5-((R)-sec-Butoxycarbonylamino)-4-methyl-[1,2,3]triazol-1-yl]-biphenyl-4-yl}-cyclopropanecarboxylic acid;
1-[4′-(5-iso-Propoxycarbonylamino-4-methyl-[1,2,3]triazol-1-yl)-biphenyl-4-yl]-cyclopropanecarboxylic acid;
1-{4′-[5-(1-Cyclopropyl-ethoxycarbonylamino)-4-methyl-[1,2,3]triazol-1-yl]-biphenyl-4-yl}-cyclopropanecarboxylic acid;
1-{4′-[5-(1-Cyclobutyl-ethoxycarbonylamino)-4-methyl-[1,2,3]triazol-1-yl]-biphenyl-4-yl}-cyclopropanecarboxylic acid;
1-[4′-(5-tert-Butoxycarbonylamino-4-methyl-[1,2,3]triazol-1-yl)-biphenyl-4-yl]-cyclopropanecarboxylic acid;
1-{3-Fluoro-4′-[4-methyl-5-((R)-1-phenyl-ethoxycarbonylamino)-[1,2,3]triazol-1-yl]-biphenyl-4-yl}-cyclopropanecarboxylic acid;
1-{3′-Methoxy-4′-[4-methyl-5-((R)-1-phenyl-ethoxycarbonylamino)-[1,2,3]triazol-1-yl]-biphenyl-4-yl}-cyclopropanecarboxylic acid;
1-{4′-[4-Ethyl-5-((R)-1-phenyl-ethoxycarbonylamino)-[1,2,3]triazol-1-yl]-biphenyl-4-yl}-cyclopropanecarboxylic acid;
{4′-[4-Ethyl-5-((R)-1-phenyl-ethoxycarbonylamino)-[1,2,3]triazol-1-yl]-biphenyl-4-yl}-acetic acid;
1-(4′-{4-Ethyl-5-[(R)-1-(3-trifluoromethyl-phenyl)-ethoxycarbonylamino]-[1,2,3]triazol-1 yl}-biphenyl-4-yl)-cyclopropanecarboxylic acid;
{4′-[4-Ethyl-5-((R)-1-(3-trifluoromethyl-phenyl-ethoxycarbonylamino)-[1,2,3]triazol-1-yl]-biphenyl-4-yl}-acetic acid;
1-{4′-[5-((R)-1-Phenyl-ethoxycarbonylamino)-[1,2,3]triazol-1-yl]-biphenyl-4-yl}-cyclopropanecarboxylic acid;
{4′-[5-((R)-1-Phenyl-ethoxycarbonylamino)-[1,2,3]triazol-1-yl]-biphenyl-4-yl}-acetic acid;
2-Methyl-2-{4′-[4-methyl-5-((R)-1-phenyl-ethoxycarbonylamino)-[1,2,3]triazol-1-yl]-biphenyl-4-yl}-propionic acid;
(R)-1-(4′-(4-Methyl-5-((1-phenylethoxy)carbonylamino)-1H-1,2,3-triazol-1-yl)biphenyl-3-yl)cyclopropanecarboxylic acid;
1-{3′-[4-Methyl-5-((R)-1-phenyl-ethoxycarbonylamino)-[1,2,3]triazol-1-yl]-biphenyl-4-yl}-cyclopropanecarboxylic acid;
{3′-[4-Methyl-5-((R)-1-phenyl-ethoxycarbonylamino)-[1,2,3]triazol-1-yl]-biphenyl-4-yl}-acetic acid;
(3-Biphenyl-4-yl-5-methyl-3H-[1,2,3]triazol-4-yl)-carbamic acid (R)-1-phenyl-ethyl ester;
[3-(4′-Cyano-biphenyl-4-yl)-5-methyl-3H-[1,2,3]triazol-4-yl]-carbamic acid (R)-1-phenyl-ethyl ester;
(R)-1-Phenyl-ethyl-1-(4′-(1-(1H-tetrazol-5-yl)cyclopropyl)biphenyl-4-yl)-4-methyl-1H-1,2,3-triazol-5-ylcarbamate;
{3-[4′-(1-Methanesulfonylaminocarbonyl-cyclopropyl)-biphenyl-4-yl]-5-methyl-3H[1,2,3]triazol-4-yl}-carbamic acid (R)-1-phenyl-ethyl ester;
1-{4′-[3-((R)-1-Phenyl-ethoxycarbonylamino)-[1,2,4]triazol-4-yl]-biphenyl-4-yl}-cyclopropanecarboxylic acid;
(R)-1-(4′-(4-Methyl-5-((1-phenylethoxy)carbonylamino)-1H-1,2,3-triazol-1-yl)biphenyl-4-yl)cyclobutanecarboxylic acid;
(R)-2-{4′-[4-Methyl-5-(-1-phenylethoxycarbonylamino)-[1,2,3]triazol-1-yl]-biphenyl-4-yl}-pent-4-enoic acid;
(R)-2-(4-(4-(4-Methyl-5-((1-phenylethoxy)carbonylamino)-1H-1,2,3-triazol-1-yl)phenyl)cyclohexyl)acetic acid; or
{3-[4′-(1-Methanesulfonylaminocarbonyl-cyclopropyl)-biphenyl-4-yl]-5-methyl-3H[1,2,3]triazol-4-yl}-carbamic acid (R)-1-(3-trifluoromethyl-phenyl)-ethyl ester.

In another embodiment of the invention, provided is a compound of formula (I) for use as a therapeutically active substance.

In another embodiment of the invention, provided is pharmaceutical composition comprising a therapeutically effective amount of a compound of formula (I) and a therapeutically inert carrier.

In another embodiment of the invention, provided is a use of a compound according to formula (I) for the treatment or prophylaxis of pulmonary fibrosis.

In another embodiment of the invention, provided is a use of a compound according to formula (I) for the preparation of a medicament for the treatment or prophylaxis of pulmonary fibrosis.

In another embodiment of the invention, provided is a compound according to formula (I) for the treatment or prophylaxis of pulmonary fibrosis.

In another embodiment of the invention, provided is a compound according formula (I), when manufactured according to a process below.

In another embodiment of the invention, provided is a method for the treatment or prophylaxis of pulmonary fibrosis, which method comprises the step of administering a therapeutically effective amount of a compound of formula (I) to a patient in need thereof.

In another embodiment of the invention, provided is an invention as hereinbefore described.

It will be appreciated, that the compounds of general formula I in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds of the present invention can be prepared beginning with commercially available starting materials, or utilizing general synthetic techniques and procedures known to those skilled in the art. Chemicals may be purchased from companies such as for example Aldrich, Argonaut Technologies, VWR, Lancaster, Princeton, Alfa, Oakwood, TCI, Fluorochem, Apollo, Matrix, Maybridge or Meinoah. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are prepacked silica gel columns used in standard chromatography. Final compounds and intermediates were named using the AutoNom2000 feature in the MDL ISIS Draw application.

The present invention is also directed to the administration of a therapeutically effective amount of a compound of formula I in combination or association with other drugs or active agents for the treatment of inflammatory or allergic diseases and disorders. In one embodiment, the present invention relates to a method for the treatment and/or prevention of such diseases or disorders comprising administering to a human or animal simultaneously, sequentially, or separately, a therapeutically effective amount of a compound of formula I and another drug or active agent (such as another anti-inflammatory or anti-allergic drug or agent). These other drugs or active agents may have the same, similar, or a completely different mode of action. Suitable other drugs or active agents may include, but are not limited to: Beta2-adrenergic agonists such as albuterol or salmeterol; corticosteroids such as dexamethasone or fluticasone; antihistamines such as loratidine; leukotriene antagonists such as montelukast or zafirlukast; anti-IgE antibody therapies such as omalizumab; antiinfectives such as fusidic acid (particularly for the treatment of atopic dermatitis); antifungals such as clotrimazole (particularly for the treatment of atopic dermatitis); immunosuppressants such as tacrolimus and pimecrolimus; other antagonists of PGD2 acting at other receptors such as DP antagonists; inhibitors of phosphodiesterase type 4 such as cilomilast; drugs that modulate cytokine production such as inhibitors of TNF-alpha converting enzyme (TACE); drugs that modulate the activity of Th2 cytokines IL-4 and IL-5 such as blocking monoclonal antibodies and soluble receptors; PPAR-gamma agonists such as rosiglitazone; and 5-lipoxygenase inhibitors such as zileuton.

The compounds of the present invention can be prepared by any conventional means. Suitable processes for synthesizing these compounds are provided in the examples. Generally, compounds of formula I can be prepared according to the schemes illustrated below. For example, certain compounds of the invention may be made using the approach outlined in Scheme 1.

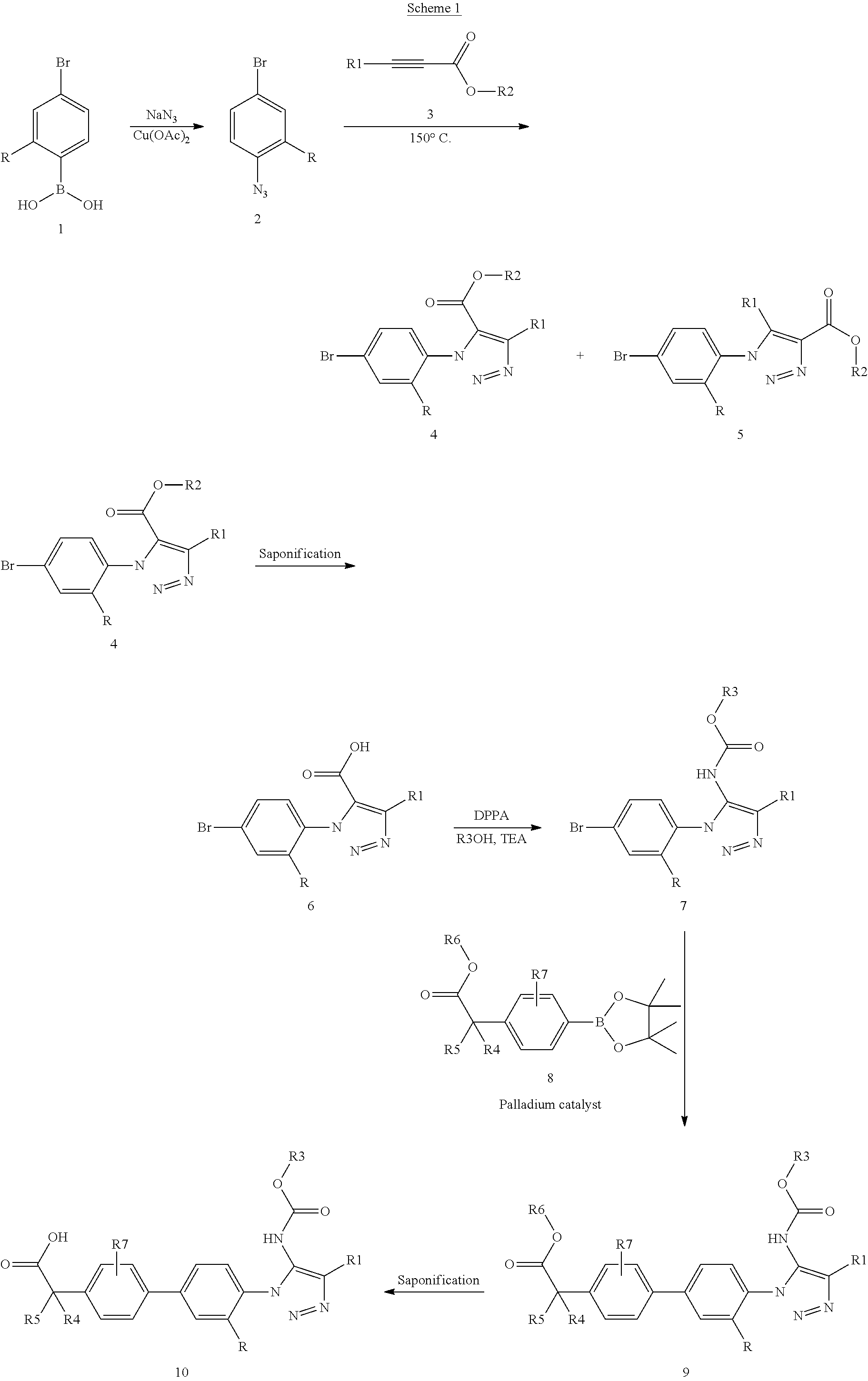

The compounds of the present invention of formula 10 can be prepared according to Scheme 1. Starting with 4-bromophenylboronic acid 1, the coupling reaction can be carried out with sodium azide in the presence of copper acetate to provide the azide intermediate 2 in protic solvents such as methanol at room temperature. The best yields can be obtained when the reaction mixture opened to the atmosphere. This azide intermediate is stable under cold conditions, but ideally it should be used immediately in the cycloaddition reaction.

The crucial 3+2 cycloaddition reaction between the azide intermediate 2 and the alkynoate 3 can be performed in toluene at higher temperature, preferably at 150° C. for 2-15 h. The reaction times can depend on the R1 groups of alkynoate, which can be hydrogen, lower alkyl, preferably methyl and ethyl groups. The ratio of both triazole regioisomers 4 and 5 depend on the R1 group and when the R1 group is methyl or ethyl the ratio generally should be 1:1.2 and when the R1 is hydrogen the ratio would be 1:4, the wrong isomer can form predominantly. The reaction temperature can be lowered if the reaction performed in the presence of a copper catalyst.

The two regioisomers can be converted to the final compounds separately. Hydrolysis of ester 4 to the corresponding acid 6 can be accomplished in the presence of a base such as lithium hydroxide in an inert solvent such as tetrahydrofuran and water at room temperature for several hours.

The acid 6 can be converted to a carbamate 7 using the Curtis rearrangement conditions such as diphenylphosphorylazide (DPPA) and a base such as triethylamine in the presence of an alcohol R3OH in an inert solvent such as toluene at 65-80° C. for several hours. The R3 can be a simple alkyl, cycloalkyl, or aryl-substituted alkyl.

The cross-coupling reaction between compounds 7 and 8 to provide the biaryl intermediate 9 can be accomplished in the presence of a palladium catalyst such as palladium(II) acetate and a phosphine ligand such as 2-dicyclohexylphosphino-2′,6′-dimethoxybiphenyl (S-Phos) in the presence of a base such as potassium phosphate tribasic in a mixture of solvents for example toluene and water. This reaction can be carried out at higher temperature, preferably at 100-105° C. for several hours.

The final compounds 10 of the invention can be obtained by hydrolysis of ester 9 in the presence of a base such as lithium hydroxide or sodium hydroxide in an inert solvent such as tetrahydrofuran, ethanol, and water at room temperature for several hours.

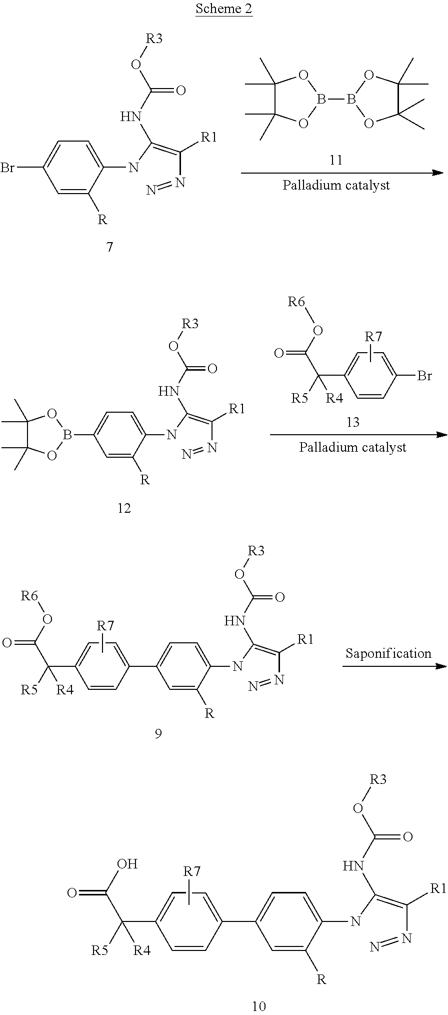

Alternatively, as described in Scheme 2, the bromo intermediate 7 can be converted to the corresponding pinacolatoboronate intermediate 12 using bispinacolatodiboron 11 in the presence of a palladium catalyst such as 1,1′-bis(diphenylphosphino)ferrocene dichloropalladium(II) in the presence of a suitable base such as potassium acetate. The preferred solvent for this reaction can be 1,4-doxane at 80° C. for several hours. The pinacolatoborane intermediate 12 then undergo a cross-coupling reaction with bromo intermediates such as 13 under palladium mediated coupling conditions to provide compound 9 which then can give the final compound 10 after treatment with regular hydrolysis conditions.

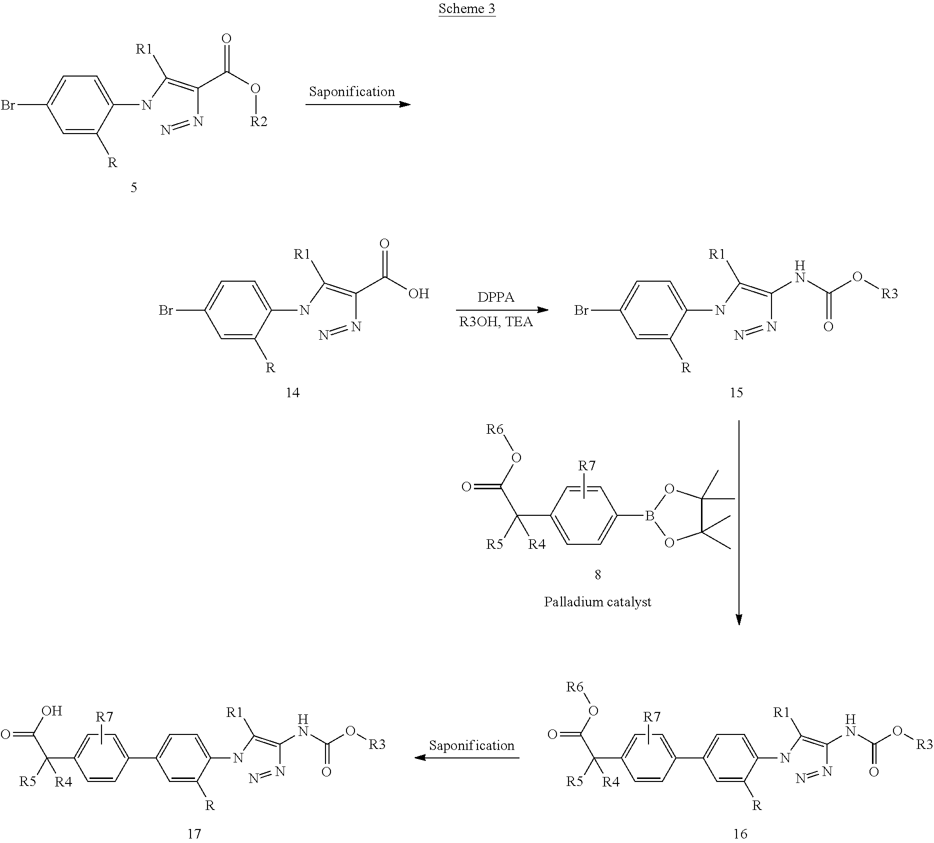

Scheme 3 described the conversion of other regioisomer 5 to the corresponding final compounds 17 following the same reaction conditions as mentioned above.

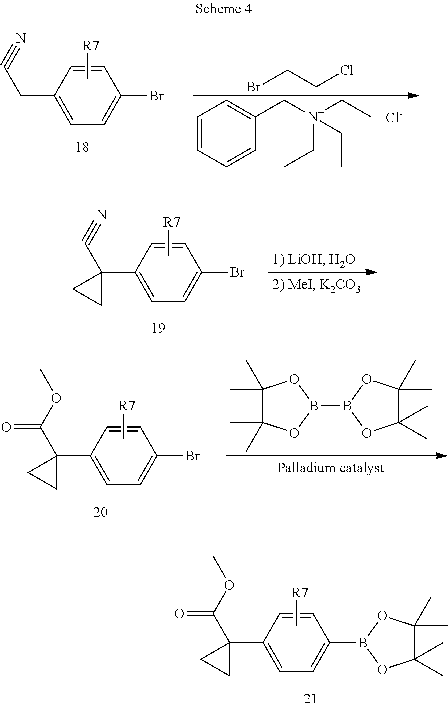

Scheme 4 described the synthesis of commercially unavailable substituted arylboronate intermediates. The 4-bromophenylacetonitrile 18 can be converted to compound 19 by treatment with 1-bromo-2-chloroethane and sodium hydroxide in the presence of a phase transfer catalyst such as benzyltriethylammonium chloride at 50° C. for several hours. Then, the cyano group of 19 can be hydrolyzed to the corresponding acid which can be treated with methyl iodide in the presence of a base such as potassium carbonate to obtain compound 20. The bromo intermediate 20 can be reacted with a bispinacolatodiboron using a palladium mediated reaction conditions to form the boronate intermediate 21.

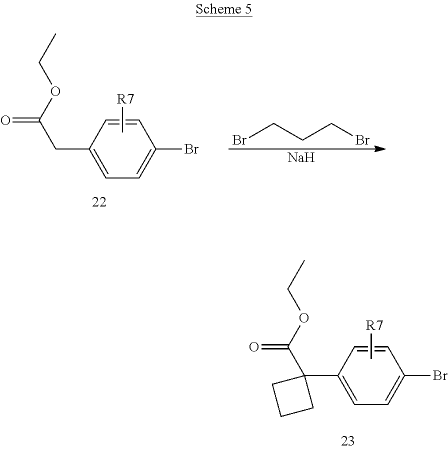

As shown in Scheme 5, the 1-(4-bromophenyl)cyclobutane or cyclopentane carboxylate intermediates such as 23 can be prepared from ethyl 2-(4-bromophenyl)acetate 22 and 1,3-dibromopropane or 1,4-dibromobutane in the presence of a strong base such as sodium hydride in aprotic solvents such as DMF at 0° C. to room temperature for several hours.

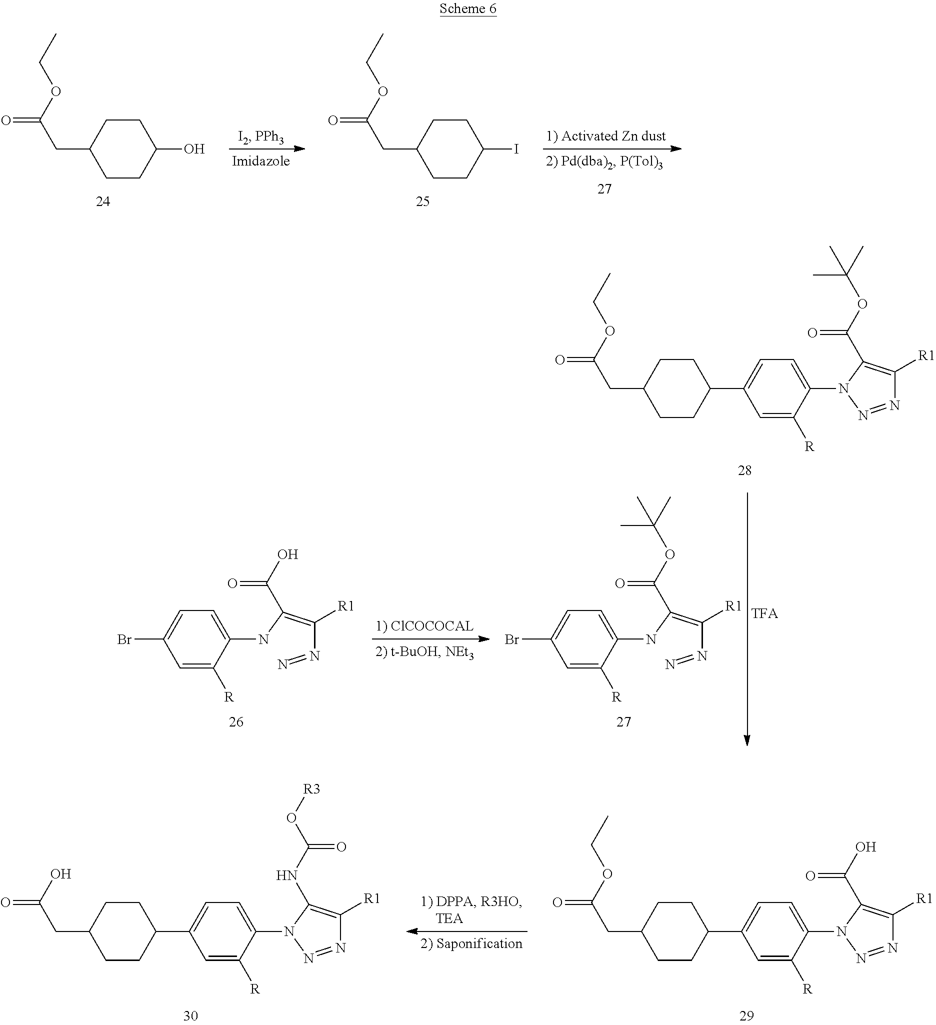

Compounds of the present invention of formula 30 can be prepared according to Scheme 6. The desired ethyl 2-(4-iodocyclohexyl)acetate 25 can be prepared from ethyl 2-(4-hydroxycyclohexyl)acetate 24 using iodine and triphenylphosphine in the presence of imidazole in dichloromethane. Then, ethyl 2-(4-iodocyclohexyl)acetate 25 can be reacted with an activated zinc dust in anhydrous THF at 60° C. for few hours to give the zinc intermediate which can undergo a cross-coupling reaction with bromo intermediate 27 in the presence of Pd(dba)₂ and tri-o-tolylphosphine in anhydrous THF at 60° C. to provide coupling product 28. The tert-butyl ester of 28 can be hydrolyzed to the acid 29 in the presence of TFA. Then, the Curtius rearrangement and saponification conditions were described in the Scheme 1 to obtain compound 30.

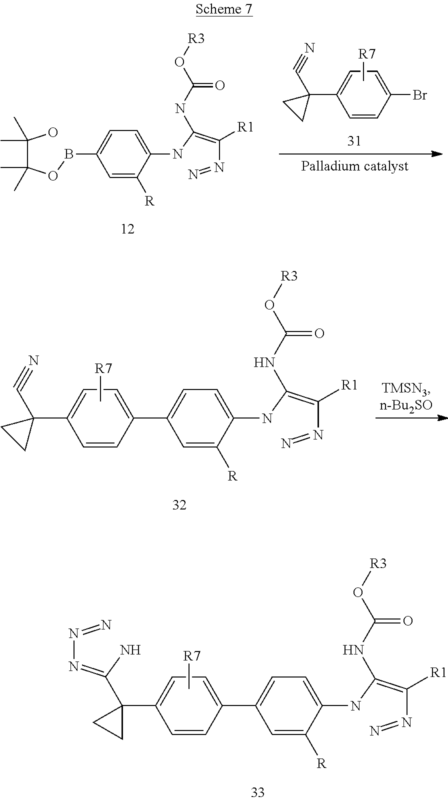

Compounds of the present invention of formula 33 can be prepared according to Scheme 7. The pinacolatoboronate intermediate 12 can be reacted with bromo intermediate 31 in the presence of a palladium catalyst such as palladium(II) acetate and a phosphine ligand such as 2-dicyclohexylphosphino-2′,6′-dimethoxybiphenyl (S-phos) in a mixed solvent system such as toluene and water at 105° C. to give compound 32. Compound 32 can be converted to the compound of interest 33 using azidotrimethylsilane and di-n-butyltin oxide in toluene at 100° C. for several hours.

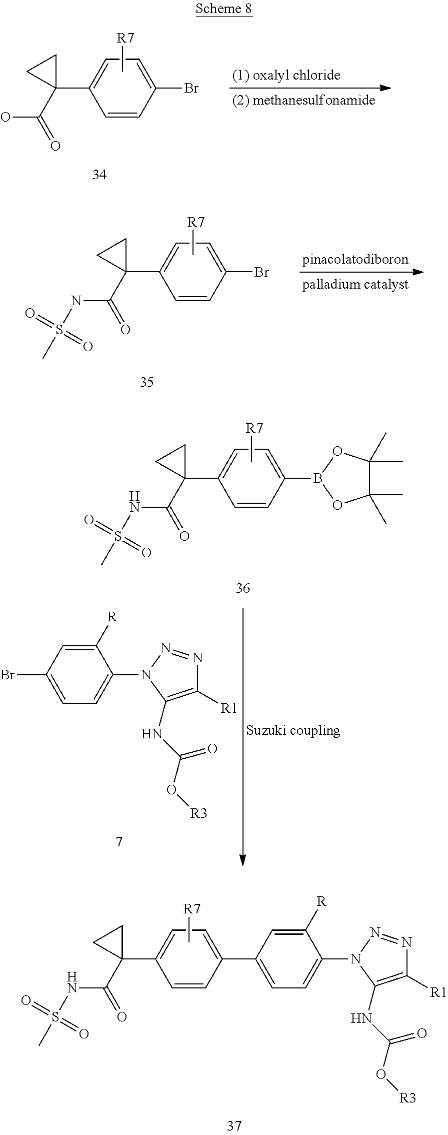

Scheme 8 described the synthesis of acyl methylsulfonamides and their resulting final compounds. The acid 34 can be converted to the acid chloride which can be reacted with methanesulfonamide in the presence of base, such as sodium hydride, to give N-acylsulfonamide 35. The arylboronate intermediate 36 can be prepared from aryl bromide 35. The final cross-coulpling step with compound 7 can be accomplished in the presence of a palladium catalyst, such as PdCl₂(dppf)CH₂Cl₂), DPPF ligand, and a base such as sodium carbonate in a mixture of solvents, for example DMF and water. This reaction can be carried out at higher temperature, preferably at 85° C. for several hours to yield the final compound 37.

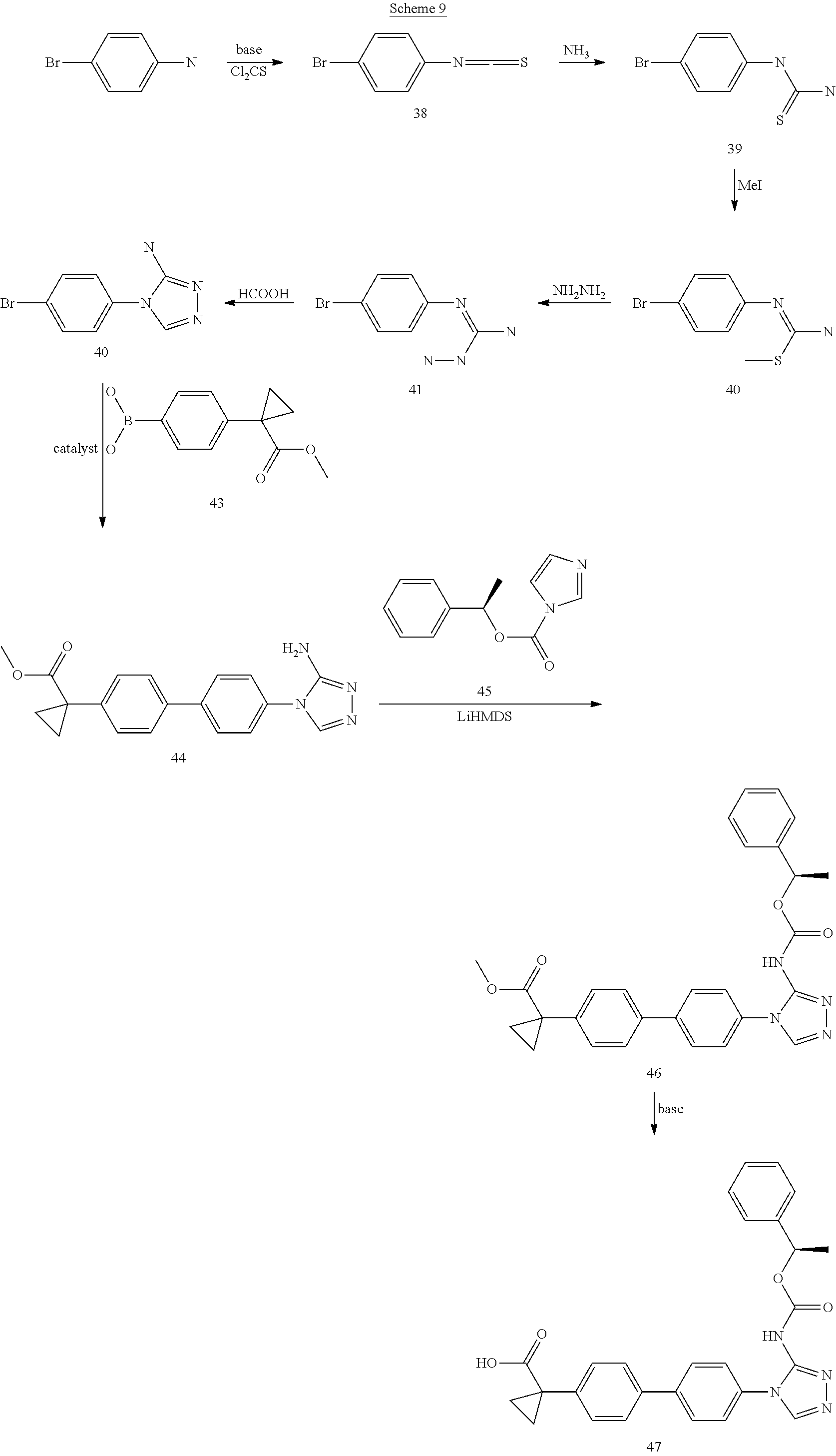

Compound of N-aryl-1,2,4-triazole derivative 47 can be prepared according to Scheme 9. 4-Bromoaniline can react with thiophosgene under basic condition to provide isothiocyanate 38, which can be converted to thiourea 39 by reacting with ammonia. Methylation of thiourea can be achieved in the presence of methyl iodide to provide the intermediate 40, which can be converted to 1N-amino-2N-arylguanidine 41 through the reaction with hydrazine. Treatment of aminoguanidine 41 with formic acid can lead to the key 4N-aryl-4H-3-amino-1,2,3-triazole 42. Under Suzuki coupling conditions, 42 can be coupled with boronic acid 43 to provide compound 44 in the presence of palladium catalyst. Compound 44 can be first deprotonated by lithium bis(hexamethyldisilyl)amide and then reacted with imidazolecarbamate 45 to provide the key carbamate 46. Imidazolecarbamate 45 can be prepared from the corresponding phenylethanol and carbonyldiimidazole (CDI). Under basic conditions, hydrolysis of 46 can lead to 4N-aryl-4H-1,2,3-triazole derivative 47. Other analogs in this chemical class can be prepared using the same method described in Scheme 9.

### EXAMPLES

Although certain exemplary embodiments are depicted and described herein, the compounds of the present invention can be prepared using appropriate starting materials according to the methods described generally herein and/or by methods available to one of ordinary skill in the art.

Definition of abbreviations: DPPA: diphenylphosphorylazide; DPPF: 1,1′-bis(diphenylphosphino)ferrocene; S-Phos: dicyclohexyl(2′,6′-dimethoxy[1,1′-biphenyl]-2-yl)-phosphine; DBA: dibenzylidineacetone; DCM: dichloromethane; DMF: dimethylformamide; EA: ethyl acetate; ACN: acetonitrile; LiHMDS: lithium bis(trimethylsilyl)amide; TEA: triethylamine; THF: tetrahydrofuran; TLC: thin layer chromatography

### Example 1

### 1-{4′-[4-Methyl-5-((R)-1-phenyl-ethoxycarbonylamino)-[1,2,3]triazol-1-yl]-biphenyl-4-yl}-cyclopropanecarboxylic acid

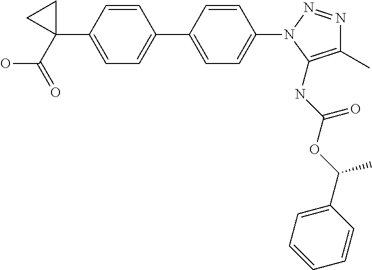 [SMILES: Cc1nnn(-c2ccc(-c3ccc(C4(C(=O)O)CC4)cc3)cc2)c1NC(=O)O[C@H](C)c1ccccc1]

### Step 1: 1-Azido-4-bromo-benzene

In a 350 mL reaction vial, 4-bromo-phenylboronic acid (21.17 g, 105 mmol), sodium azide (10.3 g, 158 mmol), and copper (II) acetate (1.91 g, 10.5 mmol) were combined with MeOH (200 mL) to give a brown suspension. The reaction was stirred at room temperature overnight open to the atmosphere, 23 hr. The reaction was concentrated, diluted with ethyl ether/hexanes (380/20 mL, first organic layer) and washed with water (100 mL, first aqueous layer) and saturated NH₄Cl/concentrate NH₄OH (200/300 mL, second aqueous layer). To the first aqueous layer was added saturated NH₄Cl and concentrated NH₄OH (60/40 mL) and the resulting organic layer was separated, washed with the second aqueous layer, and combined with the first organic layer. The first aqueous layer was extracted a second time with ether (300 mL) and the organic layer was washed with the second aqueous layer. The organic layers were combined, dried over MgSO₄ and stored in the refrigerator overnight. The crude material was warmed to room temperature, filtered, concentrated to a red/yellow oil, dissolved in hexanes (20 mL) and purified by silica gel (120 g Redisep) and eluted with hexanes to obtain 1-azido-4-bromo-benzene (19.5 g, 93.4% yield) as a yellow oil. LC/MS calcd. for C₆H₄BrN₃ (m/e) 197/199, obsd. 170/172 (M-N₂+H, ES⁺).

### Step 2: 3-(4-Bromo-phenyl)-5-methyl-3H-[1,2,3]triazole-4-carboxylic acid methyl ester

In 350 mL reaction vial 1-azido-4-bromo-benzene (10 g, 50.5 mmol) and methyl but-2-ynoate (5.45 g, 5.56 mL, 55.5 mmol) were combined with Toluene (106 mL) to give a yellow suspension. The vial was sealed and heated in an oil bath at 150° C. for 4.5 h. Cooled and stored at room temperature for 6 days. The reaction was filtered and the solid was washed with toluene and EtOAc (3×15 mL). The filtrate was concentrated, dissolved in minimal DCM, and purified by flash chromatography (silica gel, 0% to 50% EtOAc in hexanes). Appropriate fractions combined, concentrated, and dried from DCM/hexanes to give 3-(4-bromo-phenyl)-5-methyl-3H-[1,2,3]triazole-4-carboxylic acid methyl ester (4.5 g, 30.1% yield) as a light brown solid. LC/MS calcd. for C₁₁H₁₀BrN₃O₂ (m/e) 295/297, obsd. 296/298 (M+H, ES⁺).

### Step 3: 3-(4-Bromo-phenyl)-5-methyl-3H-[1,2,3]triazole-4-carboxylic acid

To 1 L round bottom flask containing 3-(4-bromo-phenyl)-5-methyl-3H-[1,2,3]triazole-4-carboxylic acid methyl ester (4.5 g, 11.5 mmol) dissolved in THF (200 mL) (brown solution) was added LiOH (2.77 g, 115 mmol) mostly dissolved in water (75 mL, with heat). The solution was stirred at room temperature for 16 h. The reaction was concentrated, diluted in water (total volume, 400 mL) extracted with ethyl ether (2×100 mL). The aqueous layer was acidified with 1 N HCl and the resulting precipitate was filtered, washed with water and hexanes, and dried over house vacuum. The white solid was partially dissolved in DCM and ACN, transferred to a round bottom flask, and dried to provide 3-(4-bromo-phenyl)-5-methyl-3H-[1,2,3]triazole-4-carboxylic acid (3.6 g, 110% yield) as an off-white solid. LC/MS calcd. for C₁₀H₈N₃O₂ (m/e) 281/283, obsd. 281/284 (M+H, ES⁺).

### Step 4: [3-(4-Bromo-phenyl)-5-methyl-3H-[1,2,3]triazol-4-yl]-carbamic acid (R)-1-phenyl-ethyl ester

In a 350 mL reaction vial, 3-(4-bromo-phenyl)-5-methyl-3H-[1,2,3]triazole-4-carboxylic acid (3.6 g, 12.8 mmol), (R)-1-phenylethanol (3.04 g, 3 mL, 24.9 mmol) and triethylamine (3.27 g, 4.5 mL, 32.3 mmol) were combined with toluene (100 mL) to give a yellow solution and to this was added diphenylphosphorazidate (8.94 g, 7 mL, 32.5 mmol). The vial's atmosphere was purged with nitrogen, sealed, heated in an oil bath at 65° C. for 2 h, and cooled to room temperature overnight. The reaction was concentrated as yellow viscous oil, diluted with DCM, and purified by flash chromatography (silica gel, 0-50% EtOAc in hexanes). Appropriate fractions combined, concentrated, dried from DCM/hexanes, to obtain [3-(4-bromo-phenyl)-5-methyl-3H-[1,2,3]triazol-4-yl]-carbamic acid (R)-1-phenyl-ethyl ester (4.07 g, 79.5% yield) as white solid. LC/MS calcd. for C₁₈H₁₇BrN₄O₂ (m/e) 400/402, obsd. 401/403 (M+H, ES⁺).

### Step 5: 1-(4-Bromo-phenyl)-cyclopropanecarbonitrile

In a 1 L round-bottomed flask, 2-(4-bromophenyl)acetonitrile (59.57 g, 304 mmol) (semi-melted at 60° C. to transfer), 1-bromo-2-chloroethane (65.4 g, 456 mmol) and N-benzyl-N,N-triethylethanaminium chloride (5.54 g, 24.3 mmol) were combined, heated in an oil bath at 50° C. to give a light brown solution. To this was added drop wise a just prepared solution of NaOH (72.9 g, 1.82 mol) in 72 mL of water (not completely dissolved). The NaOH flask and addition funnel were rinsed with water and the washings were added as well. This reaction was stirred in the oil bath at 50° C. with a condenser overnight, 22 hr. The reaction was cooled, diluted with water (500 mL), extracted with DCM (2×300 mL). The organic layers were washed with water (2×300 mL), 1 N HCl (2×300 mL) and brine (300 mL), dried over MgSO₄, filtered, concentrated, and dried yielding 1-(4-bromo-phenyl)cyclopropanecarbonitrile (66.8 g, 99% yield) as a yellow solid. LC/MS calcd. for C₁₀H₈BrN (m/e) 221/223, obsd. 222/224 (M+H, ES⁺), 263/265 (M+ACN+H, ES⁺).

### Step 6: 1-(4-Bromo-phenyl)-cyclopropanecarboxylic acid

In a 2 L round-bottomed flask, 1-(4-bromo-phenyl)cyclopropanecarbonitrile (66.8 g, 301 mmol) was combined with LiOH (144 g, 6.02 mol) partially dissolved in water (1.1 L) to give a red suspension and stirred in an oil bath heated at reflux for 7 h. The reaction was cooled to room temperature over the weekend. The off white/grey mixture was diluted with water (˜1 L) and extracted with EtOAc (2×400 mL) keeping solid in aqueous layer. The aqueous layer was acidified with concentrate HCl to pH˜3 and the resulting precipitate was filtered and washed with hexanes (4×total 0.9 L) yielding, 1-(4-bromo-phenyl)cyclopropanecarboxylic acid (73.3 g, 101% yield) as an off-white solid. LC/MS calcd. for C₁₀H₉BrO₂ (m/e) 240/242, obsd. 241/243 (M+H, ES⁺), 239/241 (M−H, ES⁻).

### Step 7: 1-(4-Bromo-phenyl)-cyclopropanecarboxylic acid methyl ester

In a 2 L round-bottomed flask, 1-(4-bromo-phenyl)cyclopropanecarboxylic acid (73.6 g, 305 mmol) was combined with DMF (0.5 L) to give a light brown/red solution and to this magnetically stirred solution was added K₂CO₃ (127 g, 916 mmol). After about 10 min a white precipitate formed and the solution became unstirrable. The material was transferred to a 3 L three-neck-flask, diluted with DMF (1 L) and magnetically stirred. To this was dripped in over 1 h methyl iodide (217 g, 95.4 mL, 1.53 mol) dissolved in DMF (0.1 L). The white suspension was stirred at room temperature overnight. The reaction was split in half, and each half was partially concentrated (removed ˜300 mL volume), diluted with water (1 L), and extracted with EtOAc (2×500 mL). Each EtOAc layer was washed with water (500 mL) and brine (250 mL), combined, dried over MgSO₄, filtered, concentrated, (combined with the other half), concentrated yielding 1-(4-bromophenyl)-cyclopropanecarboxylic acid methyl ester (73.3 g, 94.1% yield) as light brown oil. LC/MS calcd. for C₁₁H₁₁BrO₂ (m/e) 254/256, obsd. 255/257 (M+H, ES⁺).

### Step 8: 1-[4-(4,4,5,5-Tetramethyl-[1,3,2]dioxaborolan-2-yl)-phenyl]-cyclopropanecarboxylic acid methyl ester

In a 350 mL reaction vial, 1-(4-bromophenyl)-cyclopropanecarboxylic acid methyl ester (20 g, 78.4 mmol), BISPIN (23.9 g, 94.1 mmol) and potassium acetate (15.4 g, 157 mmol) were combined with 1,4-dioxane (150 mL) to give a light brown suspension. The mixture was purged with nitrogen for 5 min, PdCl₂(dppf) (3.2 g, 3.92 mmol) was added and the vial was sealed and heated in an oil bath at 80° C. for 4 hr. The reaction was filtered through celite (rinsed/DCM), concentrated, diluted ethyl ether (500 mL), washed with water (2×500 mL) and brine (250 mL). The aqueous layer had black solid and was filtered and the solid washed with ethyl ether. This filtrate was extracted with ethyl ether (500 mL) and washed with the same brine. The ethyl ether layers were combined, dried over MgSO₄, filtered, and concentrated as red oil. The crude material was purified by flash chromatography (silica gel, 0% to 20% EtOAc in hexanes). The appropriate fractions were combined, concentrated, dried from DCM to provide 1-[4-(4,4,5,5-tetramethyl-[1,3,2]dioxaborolan-2-yl)-phenyl]-cyclopropanecarboxylic acid methyl ester (20.83 g, 87.9% yield) as a white to white/very faint yellow solid. LC/MS calcd. for C₁₇H₂₃BO₄ (m/e) 302, obsd. 303 (M+H, ES⁺).

### Step 9: 1-{4′-[4-Methyl-5-((R)-1-phenyl-ethoxycarbonylamino)-[1,2,3]triazol-1-yl]-biphenyl-4-yl}-cyclopropanecarboxylic acid methyl ester

In a 350 mL vial, 1-[4-(4,4,5,5-tetramethyl-[1,3,2]dioxaborolan-2-yl)-phenyl]-cyclopropanecarboxylic acid methyl ester (2.49 g, 8.22 mmol), [3-(4-bromo-phenyl)-5-methyl-3H-[1,2,3]triazol-4-yl]-carbamic acid (R)-1-phenyl-ethyl ester (3.0 g, 7.48 mmol), 2-dicyclohexyphosphino-2′,6′-dimethoxybiphenyl (SPhos) (921 mg, 2.24 mmol), and palladium(II) acetate (252 mg, 1.12 mmol) were combined with toluene (120 mL) (previously purged with nitrogen for 20 min) to give a light yellow solution. To this was added tripotassium phosphate (4.76 g, 22.4 mmol) dissolved in water (30.0 mL) (previously purged with nitrogen for 20 min). The vial's atmosphere was replaced with nitrogen, sealed, heated in oil bath at 100° C. for 4 h, and cooled to room temperature overnight. The reaction was diluted with EtOAc (50 mL) and water (100 mL) and filtered and rinsed with water (30 mL) and EtOAc (50 mL). The filtrate was separated by addition of brine (50 mL) and the organic layer was washed with brine (150 mL). The aqueous layer was extracted with EtOAc (2×150 mL) and each organic layer was washed with the same brine. The organic layers were combined, dried over MgSO₄, filtered, concentrated, dissolved in minimal DCM and purified by flash chromatography (silica gel, 0% to 50% EtOAc in hexanes). Appropriate fractions combined, concentrated, and dried from DCM/hexanes to obtain 1-{4′-[4-Methyl-5-((R)-1-phenyl-ethoxycarbonylamino)-[1,2,3]triazol-1-yl]-biphenyl-4-yl}-cyclopropanecarboxylic acid methyl ester (2.65 g, 71.4% yield) as a white solid. LC/MS calcd. for C₂₉H₂₈N₄O₄ (m/e) 496, obsd. 497 (M+H, ES⁺).

### Step 10: 1-{4′-[4-Methyl-5-((R)-1-phenyl-ethoxycarbonylamino)-[1,2,3]triazol-1-yl]-biphenyl-4-yl}-cyclopropanecarboxylic acid

In a 1 L round-bottomed flask, 1-{4′-[4-Methyl-5-((R)-1-phenyl-ethoxycarbonylamino)[1,2,3]triazol-1-yl]-biphenyl-4-yl}-cyclopropanecarboxylic acid methyl ester (2.65 g, 5.34 mmol) was combined with THF (50 mL) to give a yellow solution. To this was dripped in LiOH (1.28 g, 53.4 mmol) dissolved in water (12.5 mL, heated to partially dissolve). The reaction flask sealed and heated in an oil bath at 60° C. for 5 h. The reaction cooled to room temperature overnight. The reaction was diluted with water (100 mL), concentrated, diluted with more water (500 mL) and acidified with 1 N HCl. The resulting precipitate was filtered, washed with water and hexanes and dried over house vacuum and in a desiccator. The crude product (2.8 g), as a white solid, was triturated from hot ACN and recrystallized from EtOAc, EtOH/water, and IPA/water. These attempted purifications were unsuccessful and the resulting solid (2.0 g) was purified by flash reverse phase chromatography (C18 Silicycle 120 g, 60 mL min 20-100% ACN/H₂O 20 min). Appropriate fractions combined, concentrated, diluted with water, and the resulting precipitate was filtered and washed with water and hexanes yielding 1.65 g of a white solid. The solid was crystallized from ACN to give 1-{4′-[4-methyl-5-((R)-1-phenyl-ethoxycarbonylamino)-[1,2,3]triazol-1-yl]-biphenyl-4-yl}-cyclopropanecarboxylic acid (1.48 g, 57.5% yield) as a white solid. LC/MS calcd. for C₂₈H₂₆N₄O₄ (m/e) 482, obsd. 483 (M+H, ES⁺). ¹H NMR (DMSO-d₆) δ: 12.40 (br. s., 1H), 9.69 (br. s., 1H), 7.83 (d, J=7.0 Hz, 2H), 7.67 (d, J=8.3 Hz, 2H), 7.58 (d, J=8.0 Hz, 2H), 7.47 (d, J=8.5 Hz, 2H), 7.00-7.42 (m, 5H), 5.71 (br. s., 1H), 2.18 (s, 3H), 1.29-1.69 (m, 5H), 1.13-1.26 (m, 2H).

### Example 2

### {4′-[4-Methyl-5-((R)-1-phenyl-ethoxycarbonylamino)-[1,2,3]triazol-1-yl]-biphenyl-4-yl}-acetic acid

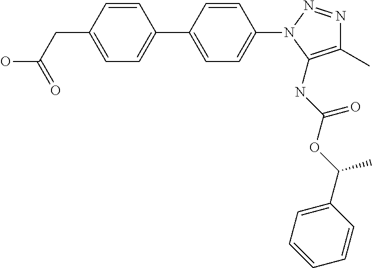 [SMILES: Cc1nnn(-c2ccc(-c3ccc(CC(=O)O)cc3)cc2)c1NC(=O)O[C@H](C)c1ccccc1]

### Step 1: [4-(4,4,5,5-Tetramethyl-[1,3,2]dioxaborolan-2-yl)-phenyl]-acetic acid ethyl ester

In a 350 mL reaction vial, ethyl 2-(4-bromophenyl)acetate (25 g, 103 mmol), BISPIN (31.3 g, 123 mmol) and potassium acetate (20.2 g, 206 mmol) were combined with 1,4 dioxane (190 mL) to give a white suspension. The mixture was purged with nitrogen for 5 min, PdCl₂(dppf) (4.2 g, 5.14 mmol) was added and the vial was sealed and heated in an oil bath at 80° C. for 3 h. The reaction was filtered, rinsed with ethyl ether, concentrated, diluted with water (500 mL) and extracted with ethyl ether (2×300 mL), and the organic layers washed with brine (250 mL). The ethyl ether layers were combined, dried over MgSO₄, filtered, and concentrated as red oil. The crude material was purified by flash chromatography (silica gel, 0% to 20% EtOAc in hexanes). The appropriate fractions were combined, concentrated, dried from DCM to obtain [4-(4,4,5,5-tetramethyl-[1,3,2]dioxaborolan-2-yl)-phenyl]-acetic acid ethyl ester (25.14 g, 84.2% yield) as a white solid/oil. LC/MS calcd. for C₁₆H₂₃BO₄ (m/e) 290, obsd. 291 (M+H, ES⁺).

### Step 2: {4′-[4-Methyl-5-((R)-1-phenyl-ethoxycarbonylamino)-[1,2,3]triazol-1-yl]-biphenyl-4-yl}-acetic acid ethyl ester

In a 20 mL vial, [4-(4,4,5,5-tetramethyl-[1,3,2]dioxaborolan-2-yl)-phenyl]-acetic acid ethyl ester (79.5 mg, 0.274 mmol), [1-(4-bromo-phenyl)-5-methyl-1H-[1,2,3]triazol-4-yl]-carbamic acid (R)-1-phenyl-ethyl ester (100 mg, 0.249 mmol), tripotassium phosphate (159 mg, 0.748 mmol), 2-dicyclohexyphosphino-2′,6′-dimethoxybiphenyl (SPhos) (30.7 mg, 0.0748 mmol), and palladium(II) acetate (8.4 mg, 0.037 mmol) were combined with toluene (2 mL) and water (0.5 mL) (previously purged with nitrogen for 20 min) to give a light yellow suspension. The vial's atmosphere was replaced with nitrogen, sealed, heated in a dry block at 100° C. for 6 h, and cooled to room temperature overnight. The reaction was diluted with EtOAc (50 mL) and washed with water (50 mL) and brine. The aqueous layers were extracted with EtOAc (50 mL). The organic layers were combined, dried over MgSO₄, filtered, concentrated, dissolved in minimal DCM and purified by flash chromatography (silica gel, 0% to 60% EtOAc in hexanes). Appropriate fractions combined, concentrated, and dried from DCM/hexanes to obtain {4′-[4-methyl-5-((R)-1-phenyl-ethoxycarbonylamino)-[1,2,3]triazol-1-yl]-biphenyl-4-yl}-acetic acid ethyl ester (40 mg, 0.0826 mmol, 33.1% yield) as a colorless waxy solid. LC/MS calcd. for C₂₈H₂₈N₄O₄ (m/e) 484, obsd. 485 (M+H, ES⁺).

### Step 3: {4′-[4-Methyl-5-((R)-1-phenyl-ethoxycarbonylamino)-[1,2,3]triazol-1-yl]-biphenyl-4-yl}-acetic acid

In a 200 mL round-bottomed flask, {4′-[4-methyl-5-((R)-1-phenyl-ethoxycarbonylamino)[1,2,3]triazol-1-yl]-biphenyl-4-yl}-acetic acid ethyl ester (34 mg, 0.0702 mmol) was combined with THF (2 mL) to give a yellow solution. To this was dripped in LiOH (16.8 mg, 0.702 mmol) dissolved in water (0.5 mL, heated to partially dissolve). The reaction flask sealed and heated in an oil bath at 60° C. for 11 h. The reaction cooled to room temperature, diluted with water, and acidified with 1 N HCl. The resulting precipitate was filtered, washed with water, and hexanes and dried over house vacuum yielding 1-{4′-[5-methyl-4-((R)-1-phenyl-ethoxycarbonylamino)-[1,2,3]triazol-1-yl]-biphenyl-4-yl}-cyclopropanecarboxylic acid (40 mg, 62.4% yield) as an off-white solid. LC/MS calcd. for C₂₆H₂₄N₄O₄ (me) 456, obsd. 457 (M+H, ES⁺). ¹H NMR (DMSO-d₆) δ: 12.42 (br. s., 1H), 9.19-9.80 (m, 1H), 7.83 (d, J=6.5 Hz, 2H), 7.69 (d, J=8.0 Hz, 2H), 7.57 (d, J=7.3 Hz, 2H), 7.08-7.47 (m, 7H), 5.69 (br. s., 1H), 3.65 (s, 2H), 2.16 (s, 3H), 1.13-1.64 (m, 3H)

### Example 3

### 1-{4′-[5-Methyl-4-((R)-1-phenyl-ethoxycarbonylamino)-[1,2,3]triazol-1-yl]-biphenyl-4-yl}-cyclopropanecarboxylic acid

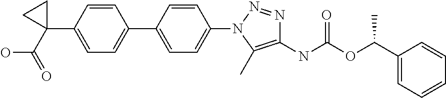 [SMILES: Cc1c(NC(=O)O[C@H](C)c2ccccc2)nnn1-c1ccc(-c2ccc(C3(C(=O)O)CC3)cc2)cc1]

### Step 1: 1-(4-Bromo-phenyl)-5-methyl-1H-[1,2,3]triazole-4-carboxylic acid methyl ester

In 20 mL reaction vial, 1-azido-4-bromo-benzene (1.647 g, 8.32 mmol and methyl but-2-ynoate (0.816 g, 0.8 mL, 8.32 mmol) were combined with Toluene (15 mL) to give a yellow solution. The vial's atmosphere was purged with nitrogen, the vial sealed, and microwaved at 150° C. for 1 h. The resulting solid in the reaction was filtered, and washed with toluene yielding 1-(4-Bromo-phenyl)-5-methyl-1H-[1,2,3]triazole-4-carboxylic acid methyl ester (0.33 g, 1.11 mmol, 13.3% yield). The filtrate was concentrated, transferred to a reaction vial with toluene (10 mL), methyl but-2-ynoate (816 mg, 0.8 mL, 8.32 mmol) was added, and the reaction was performed the same as above. The reaction with minimal solid was supported on silica gel and purified by flash chromatography (silica gel, 0% to 40% EtOAc in hexanes). Appropriate fractions were combined, concentrated, and dried from DCM/hexanes yielding 1-(4-Bromo-phenyl)-5-methyl-1H-[1,2,3]triazole-4-carboxylic acid methyl ester (0.87 g, 35.3% yield) as a light brown solid. The precipitate from the reaction was not combined with the isolated product from the column. LC/MS calcd. for C₁₁H₁₀BrN₃O₂ (m/e) 295/297, obsd. 296/298 (M+H, ES⁺).

### Step 2: 1-(4-Bromo-phenyl)-5-methyl-1H-[1,2,3]triazole-4-carboxylic acid

To 500 mL round bottom flask containing 1-(4-bromo-phenyl)-5-methyl-1H-[1,2,3]triazole-4-carboxylic acid methyl ester (0.87 g, 2.9 mmol) dissolved in THF (30 mL) (brown solution) was added LiOH (0.71 g, 30 mmol) mostly dissolved in water (7 mL, with heat). The solution was stirred at room temperature overnight. The reaction was concentrated, diluted in water (total volume, 100 mL), and extracted with ethyl ether (2×100 mL). The aqueous layer was acidified with 1 N HCl. The resulting precipitate was filtered, washed with water and hexanes, and dried over house vacuum and on lypholizer to obtain 1-(4-bromo-phenyl)-5-methyl-1H-[1,2,3]triazole-4-carboxylic acid (3.6 g, 110% yield) as a brown solid. LC/MS calcd. for C₁₀H₈N₃O₂ (m/e) 281/283, obsd. 281/284 (M+H, ES⁺).

### Step 3: [1-(4-Bromo-phenyl)-5-methyl-1H-[1,2,3]triazol-4-yl]-carbamic acid (R)-1-phenyl-ethyl ester

In a 20 mL reaction vial, 1-(4-bromo-phenyl)-5-methyl-1H-[1,2,3]triazole-4-carboxylic acid (0.67 g, 2.38 mmol), (R)-1-phenylethanol (0.29 g, 0.29 mL, 2.4 mmol) and triethylamine (0.24 g, 0.33 mL, 2.4 mmol) were combined with toluene (100 mL) to give a yellow solution and to this was added diphenylphosphorylazide (0.65 g, 0.5 mL, 2.4 mmol). The vial's atmosphere was purged with nitrogren, sealed, heated in a dry block at 80° C. for 4 h, and cooled to room temperature overnight. The reaction was diluted with EtOAc (100 mL) and washed with water (100 mL) and brine (50 mL). The aqueous layers were extracted with EtOAc (100 mL). The organic layers were combined, dried over MgSO₄, filtered, concentrated, dissolved in minimal DCM, and purified by flash chromatography (silica gel, 0% to 25% EtOAc in hexanes). Appropriate fractions combined, concentrated, dried from DCM/hexanes, to give [1-(4-bromo-phenyl)-5-methyl-1H-[1,2,3]triazol-4-yl]-carbamic acid (R)-1-phenyl-ethyl ester (0.507 g, 53.2% yield) as an off-white solid. LC/MS calcd. for C₁₈H₁₇BrN₄O₂ (m/e) 400/402, obsd. 401/403 (M+H, ES⁺).

### Step 4: 1-{4′-[5-Methyl-4-((R)-1-phenyl-ethoxycarbonylamino)-[1,2,3]triazol-1-yl]-biphenyl-4-yl}-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (75.3 mg, 0.249 mmol), [1-(4-bromo-phenyl)-5-methyl-1H-[1,2,3]triazol-4-yl]-carbamic acid (R)-1-phenyl-ethyl ester (100 mg, 0.249 mmol), tripotassium phosphate (159 mg, 0.748 mmol), 2-dicyclohexyphosphino-2′,6′-dimethoxybiphenyl (SPhos) (30.7 mg, 0.0748 mmol), and Pd(OAc)₂ (8.4 mg, 0.037 mmol) were combined with toluene (2 mL) and water (0.5 mL) (previously purged with nitrogen for 20 min) to give a light yellow solution. The vial's atmosphere was purged with nitrogen, sealed, heated in a dry block at 100° C. for 5 h, and cooled to room temperature overnight. The reaction was diluted with EtOAc (50 mL) and washed with water (50 mL) and brine. The aqueous layers were extracted with EtOAc (50 mL). The organic layers were combined, dried over MgSO₄, filtered, concentrated, dissolved in minimal DCM and purified by flash chromatography (silica gel, 0% to 100% EtOAc in hexanes). Appropriate fractions combined, concentrated, and dried from DCM/hexanes to give 1-{4′-[5-methyl-4-((R)-1-phenyl-ethoxycarbonylamino)-[1,2,3]triazol-1-yl]-biphenyl-4-yl}-cyclopropanecarboxylic acid methyl ester (74 mg, 59.8% yield) as a white solid. LC/MS calcd. for C₂₉H₂₈N₄O₄ (m/e) 496, obsd. 497 (M+H, ES⁺).

### Step 5: 1-{4′-[5-Methyl-4-((R)-1-phenyl-ethoxycarbonylamino)-[1,2,3]triazol-1-yl]-biphenyl-4-yl}-cyclopropanecarboxylic acid

In a 200 mL round-bottomed flask, 1-{4′-[5-methyl-4-((R)-1-phenyl-ethoxycarbonylamino)[1,2,3]triazol-1-yl]-biphenyl-4-yl}-cyclopropanecarboxylic acid methyl ester (66 mg, 0.133 mmol) was combined with THF (3 mL) to give a yellow solution. To this was dripped in LiOH (31.8 mg, 1.334 mmol) in water (1 mL), partially dissolved with heat. The reaction flask sealed and heated in an oil bath at 60° C. for 11 h. The reaction cooled to room temperature diluted with water and acidified with 1 N HCl. The resulting precipitate was filtered, washed with water, ethyl ether, and hexanes and dried over house vacuum yielding 1-{4′-[5-methyl-4-((R)-1-phenyl-ethoxycarbonylamino)-[1,2,3]triazol-1-yl]-biphenyl-4-yl}-cyclopropanecarboxylic acid (40 mg, 62.4% yield) as an off-white solid. LC/MS calcd. for C₂₈H₂₆N₄O₄ (m/e) 482, obsd. 483 (M+H, ES⁺). ¹H NMR (DMSO-d₆) δ: 12.39 (br. s., 1H), 9.59 (br. s., 1H), 7.89 (d, J=8.5 Hz, 2H), 7.69 (dd, J=8.3, 5.3 Hz, 4H), 7.28-7.49 (m, 7H), 5.79 (q, J=6.5 Hz, 1H), 2.21 (s, 3H), 1.43-1.63 (m, 5H), 1.14-1.25 (m, 2H).

### Example 4

### {4′-[5-Methyl-4-((R)-1-phenyl-ethoxycarbonylamino)-[1,2,3]triazol-1-yl]-biphenyl-4-yl}-acetic

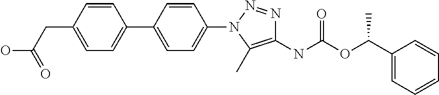 [SMILES: Cc1c(NC(=O)O[C@H](C)c2ccccc2)nnn1-c1ccc(-c2ccc(CC(=O)O)cc2)cc1]

### Step 1: {4′-[5-Methyl-4-((R)-1-phenyl-ethoxycarbonylamino)-[1,2,3]triazol-1-yl]-biphenyl-4-yl}-acetic acid methyl ester

In a 20 mL vial, [4-(4,4,5,5-tetramethyl-[1,3,2]dioxaborolan-2-yl)-phenyl]-acetic acid ethyl ester (86.8 mg, 0.299 mmol), [1-(4-bromo-phenyl)-5-methyl-1H-[1,2,3]triazol-4-yl]-carbamic acid (R)-1-phenyl-ethyl ester (100 mg, 0.249 mmol), tripotassium phosphate (159 mg, 0.748 mmol), 2-dicyclohexyphosphino-2′,6′-dimethoxybiphenyl (SPhos) (30.7 mg, 0.0748 mmol), and Pd(OAc)₂ (8.4 mg, 0.037 mmol) were combined with Toluene (2 mL) and water (0.5 mL) (previously purged with nitrogen for 20 min) to give a light yellow solution. The vial's atmosphere was purged with nitrogen, sealed, heated in a dry block at 100° C. for 16 h, and cooled to room temperature overnight. The reaction was filtered through celite, concentrated, dissolved in DCM/EtOAc/MeOH, supported on silica gel and purified by flash chromatography (silica gel, 0% to 40% EtOAc in hexanes). Appropriate fractions combined, concentrated, and dried from DCM/hexanes to give {4′-[5-methyl-4-((R)-1-phenyl-ethoxycarbonylamino)-[1,2,3]triazol-1-yl]-biphenyl-4-yl}-acetic acid methyl ester (56.6 mg, 54.3% yield) as a white solid. LC/MS calcd. for C₂₈H₂₈N₄O₄ (m/e) 484, obsd. 485 (M+H, ES⁺).

### Step 2: {4′-[5-Methyl-4-((R)-1-phenyl-ethoxycarbonylamino)-[1,2,3]triazol-1-yl]-biphenyl-4-yl}-acetic acid

In a 200 mL round-bottomed flask, {4′-[5-methyl-4-((R)-1-phenyl-ethoxycarbonylamino)[1,2,3]triazol-1-yl]-biphenyl-4-yl}-acetic acid methyl ester (59 mg, 0.122 mmol) was combined with THF (3 mL) to give a yellow solution. To this was dripped in LiOH (29.2 mg, 1.22 mmol) in water (1 mL), heated to partially dissolve. The reaction flask was sealed, heated in an oil bath at 60° C. for 3.5 h, and cooled to room temperature overnight. The reaction was diluted with water and acidified with 1 N HCl. The resulting precipitate was extracted with EtOAc (2×75 mL). The organic layers were washed with brine (50 mL), combined, dried over MgSO₄, filtered, concentrated, and dried from DCM/hexanes yielding {4′-[5-methyl-4-((R)-1-phenyl-ethoxycarbonylamino)-[1,2,3]triazol-1-yl]-biphenyl-4-yl}-acetic acid (50 mg, 90% yield) as an off-white solid. LC/MS calcd. for C₂₆H₂₄N₄O₄ (m/e) 456, obsd. 457 (M+H, ES⁺). ¹H NMR (DMSO-d₆) δ: 12.44 (br. s., 1H), 9.62 (br. s., 1H), 7.95 (d, J=8.5 Hz, 2H), 7.76 (t, J=8.0 Hz, 4H), 7.22-7.59 (m, 7H), 5.85 (q, J=6.5 Hz, 1H), 3.71 (s, 2H), 2.27 (s, 3H), 1.60 (d, J=6.0 Hz, 3H).

### Example 5

### 1-(4′-{5-[(R)-1-(2-Fluoro-phenyl)-ethoxycarbonylamino]-4-methyl-[1,2,3]triazol-1-yl}-biphenyl-4-yl)-cyclopropanecarboxylic acid

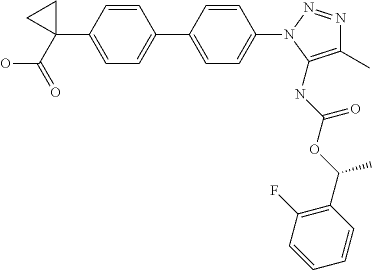 [SMILES: Cc1nnn(-c2ccc(-c3ccc(C4(C(=O)O)CC4)cc3)cc2)c1NC(=O)O[C@H](C)c1ccccc1F]

### Step 1: [3-(4-Bromo-phenyl)-5-methyl-3H-[1,2,3]triazol-4-yl]-carbamic acid (R)-1-(2-fluoro-phenyl)-ethyl ester

In a 20 mL reaction vial, 3-(4-bromo-phenyl)-5-methyl-3H-[1,2,3]triazole-4-carboxylic acid 100 mg, 0.354 mmol), (R)-1-(2-fluorophenyl)ethanol (49.6 mg, 49 μL, 0.354 mmol) and triethylamine (35.8 mg, 49.3 μL, 0.354 mmol) were combined with toluene (2.5 mL) to give a yellow suspension and to this was added diphenylphosphorylazide (97.4 mg, 76.3 μL, 0.354 mmol). The vial's atmosphere was purged with nitrogen, sealed, heated in an oil bath at 80° C. for 4 h, and cooled to room temperature overnight. Additional reagents were added, (R)-1-(2-fluorophenyl)ethanol (24.8 mg, 24.5 μL, 0.177 mmol), triethylamine (72.6 mg, 100 μL, 0717 mmol), and diphenylphosphorylazide (97.4 mg, 76.3 μL, 0.354 mmol). The vial's atmosphere was purged with nitrogen, sealed, heated in an oil bath at 80° C. for 2 h, and cooled to room temperature. The reaction was supported on celite and purified by flash chromatography (silica gel, 12 g Redisep, 20 mL/min, 0% to 40% EtOAc in hexanes). Appropriate fractions combined, concentrated, dried from DCM/hexanes, to obtain [3-(4-Bromo-phenyl)-5-methyl-3H-[1,2,3]triazol-4-yl]-carbamic acid (R)-1-(2-fluoro-phenyl)ethyl ester (95.7 mg, 64.4% yield) as solid. LC/MS calcd. for C₁₈H₁₆BrFN₄O₂ (m/e) 418/420, obsd. 419/421 (M+H, ES⁺).

### Step 2: 1-(4′-{5-[(R)-1-(2-Fluoro-phenyl)-ethoxycarbonylamino]-4-methyl-[1,2,3]triazol-1-yl}-biphenyl-4-yl)-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (74.4 mg, 0.246 mmol), [3-(4-Bromo-phenyl)-5-methyl-3H-[1,2,3]triazol-4-yl]-carbamic acid (R)-1-(2-fluoro-phenyl)-ethyl ester (86 mg, 0.205 mmol), tripotassium phosphate (131 mg, 0.615 mmol), 2-dicyclohexyphosphino-2′,6′-dimethoxybiphenyl (SPhos) (25.3 mg, 0.0615 mmol), and Pd(OAc)₂ (6.91 mg, 0.0308 mmol) were combined with toluene (2 mL) and Water (0.5 mL) (previously purged with nitrogen for 20 min) to give a light yellow suspension. The vial's atmosphere was purged with nitrogen, sealed, heated in oil bath at 100° C. for 4 h, and cooled to room temperature overnight. The reaction was diluted with EtOAc (8 mL), filtered through celite, rinsed with EtOAc (2×6 mL), dried, dissolved in minimal DCM, and purified by flash chromatography (silica gel, 0% to 50% EtOAc in hexanes). Appropriate fractions combined, concentrated, and dried from DCM/hexanes yielding 1-(4′-{5-[(R)-1-(2-fluoro-phenyl)-ethoxycarbonylamino]-4-methyl-[1,2,3]triazol-1-yl}-biphenyl-4-yl)-cyclopropanecarboxylic acid methyl ester (47.8 mg, 45.3% yield) as a white solid. LC/MS calcd. for C₂₉H₂₇FN₄O₄ (m/e) 514, obsd. 515 (M+H, ES⁺).

### Step 3: 1-(4′-{5-[(R)-1-(2-Fluoro-phenyl)-ethoxycarbonylamino]-4-methyl-[1,2,3]triazol-1-yl}-biphenyl-4-yl)-cyclopropanecarboxylic acid

In a 20 mL round-bottomed flask, 1-(4′-{5-[(R)-1-(2-fluoro-phenyl)-ethoxycarbonylamino]-4-methyl-[1,2,3]triazol-1-yl}-biphenyl-4-yl)-cyclopropanecarboxylic acid methyl ester (42 mg, 0.0816 mmol) was combined with THF (2 mL) to give a yellow solution. To this was dripped in LiOH (34.3 mg, 0.816 mmol) in water (0.5 mL) heated to partially dissolve. The vial was sealed, heated in an oil bath at 60° C. for 11 h, and cooled to room temperature overnight. The reaction was diluted with water (35 mL) and acidified with 1 N HCl. The resulting precipitate was filtered, washed with water and hexanes, and dried over house vacuum and in a desiccator to produce 1-(4′-{5-[(R)-1-(2-fluoro-phenyl)ethoxycarbonylamino]-4-methyl-[1,2,3]triazol-1-yl}-biphenyl-4-yl)-cyclopropanecarboxylic acid (44 mg, 108% yield) as a white solid. LC/MS calcd. for C₂₈H₂₅FN₄O₄ (m/e) 500, obsd. 501 (M+H, ES⁺). ¹H NMR (DMSO-d₆) δ: 12.39 (br. s., 1H), 9.74 (br. s., 1H), 7.84 (d, J=6.5 Hz, 2H), 7.67 (d, J=8.0 Hz, 2H), 7.58 (d, J=8.0 Hz, 2H), 7.47 (d, J=8.0 Hz, 2H), 6.69-7.42 (m, 4H), 5.89 (br. s., 1H), 2.17 (br. s., 3H), 1.26-1.74 (m, 5H), 1.14-1.24 (m, 2H).

### Example 6

### 1-(4′-{4-Methyl-5-[(R)-1-(2-trifluoromethyl-phenyl)-ethoxycarbonylamino]-[1,2,3]triazol-1-yl}-biphenyl-4-yl)-cyclopropanecarboxylic acid

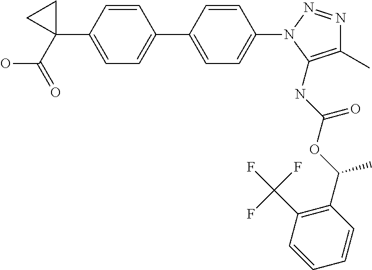 [SMILES: Cc1nnn(-c2ccc(-c3ccc(C4(C(=O)O)CC4)cc3)cc2)c1NC(=O)O[C@H](C)c1ccccc1C(F)(F)F]

### Step 1: [3-(4-Bromo-phenyl)-5-methyl-3H-[1,2,3]triazol-4-yl]-carbamic acid (R)-1-(2-trifluoromethyl-phenyl)-ethyl ester

In a 20 mL reaction vial, 3-(4-bromo-phenyl)-5-methyl-3H-[1,2,3]triazole-4-carboxylic acid 100 mg, 0.354 mmol), (R)-1-(2-trifluoromethyl-phenyl)ethanol (67.4 mg, 0.354 mmol) and triethylamine (35.8 mg, 49.3 μL, 0.354 mmol) were combined with toluene (2.5 mL) to give a yellow suspension and to this was added diphenylphosphorylazide (97.4 mg, 76.3 μL, 0.354 mmol). The vial's atmosphere was purged with nitrogren, sealed, heated in an oil bath at 80° C. for 4 h, and cooled to room temperature overnight. Additional reagents were added, (R)-1-(2-trifluoromethyl-phenyl)ethanol (33.7 mg, 0.177 mmol), triethylamine (72.6 mg, 100 μL, 0717 mmol), and diphenylphosphorylazide (97.4 mg, 76.3 μL, 0.354 mmol). The vial's atmosphere was purged with nitrogen, sealed, heated in an oil bath at 80° C. for 2 h, and cooled to room temperature. The reaction was supported on celite and purified by flash chromatography (silica gel, 12 g Redisep, 20 mL/min, 0% to 40% EtOAc in hexanes). Appropriate fractions combined, concentrated, dried from DCM/hexanes, to give [3-(4-bromo-phenyl)-5-methyl-3H-[1,2,3]triazol-4-yl]-carbamic acid (R)-1-(2-trifluoromethyl-phenyl)-ethyl ester (99.7 mg, 59.9% yield) as an off-white solid. LC/MS calcd. for C₁₉H₁₆BrF₃N₄O₂ (m/e) 468/470, obsd. 469/471 (M+H, ES⁺).

### Step 2: 1-(4′-{5-[(R)-1-(2-Trifluoromethyl-phenyl)-ethoxycarbonylamino]-4-methyl-[1,2,3]triazol-1-yl}-biphenyl-4-yl)-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (69.4 mg, 0.230 mmol), [3-(4-Bromo-phenyl)-5-methyl-3H-[1,2,3]triazol-4-yl]-carbamic acid (R)-1-(2-trifluoromethyl-phenyl)-ethyl ester (90 mg, 0.192 mmol), tripotassium phosphate (122 mg, 0.575 mmol), 2-dicyclohexyphosphino-2′,6′-dimethoxybiphenyl (SPhos) (23.6 mg, 0.0575 mmol), and Pd(OAc)₂ (6.5 mg, 0.0288 mmol) were combined with toluene (2 mL) and water (0.5 mL) (previously purged with nitrogen for 20 min) to give a light yellow suspension. The vial's atmosphere was purged with nitrogen, sealed, heated in oil bath at 100° C. for 4 h, and cooled to room temperature overnight. The reaction was diluted with EtOAc (8 mL), filtered through celite, rinsed with EtOAc (2×6 mL), dried, dissolved in minimal DCM and purified by flash chromatography (silica gel, 0% to 50% EtOAc in hexanes). Appropriate fractions combined, concentrated, and dried from DCM/hexanes yielding 1-(4′-{5-[(R)-1-(2-trifluoromethyl-phenyl)-ethoxycarbonylamino]-4-methyl-[1,2,3]triazol-1-yl}-biphenyl-4-yl)cyclopropanecarboxylic acid methyl ester (50.3 mg, 46.5% yield) as a white solid. LC/MS calcd. for C₃₀H₂₇F₃N₄O₄ (m/e) 564, obsd. 565 (M+H, ES⁺).

### Step 3: 1-(4′-{5-[(R)-1-(2-Trifluoromethyl-phenyl)-ethoxycarbonylamino]-4-methyl-[1,2,3]triazol-1-yl}-biphenyl-4-yl)-cyclopropanecarboxylic acid

In a 20 mL round-bottomed flask, 1-(4′-{5-[(R)-1-(2-trifluoromethyl-phenyl)ethoxycarbonylamino]-4-methyl-[1,2,3]triazol-1-yl}-biphenyl-4-yl)-cyclopropanecarboxylic acid methyl ester (45 mg, 0.0797 mmol) was combined with THF (2 mL) to give a yellow solution. To this was dripped in LiOH (33.5 mg, 0.797 mmol) in water (0.5 mL) heated to partially dissolve. The vial was sealed and heated in an oil bath at 60° C. for 11 h, and cooled to room temperature overnight. The reaction was diluted with water (35 mL) and acidified with 1 N HCl. The resulting precipitate was extracted into the organic layer with EtOAc (2×30 mL), washed with brine (30 mL), dried or MgSO₄, filtered, concentrated, and dried from DCM/hexanes, yielding 38.8 mg of impure product. The product was purified by RP-HPLC (Gilson, Pursuit 10 μm, 20×100 mm C18, 30 ml/min, 30 to 100% ACN/H2O, 8 min). Appropriate fractions combined, concentrated, and dried from DCM/hexanes. The product was dissolved in DCM and precipitated with addition of hexanes. The solid was filtered off and washed with hexanes, dried over house vacuum yielding 1-(4′-{5-[(R)-1-(2-trifluoromethyl-phenyl)-ethoxycarbonylamino]-4-methyl-[1,2,3]triazol-1-yl}-biphenyl-4-yl)cyclopropanecarboxylic acid (17.2 mg, 39.2% yield) as an off-white solid. LC/MS calcd. for C₂₉H₂₅F₃N₄O₄ (m/e) 550, obsd. 551 (M+H, ES⁺). ¹H NMR (DMSO-d₆) δ: 12.40 (br. s., 1H), 9.77 (br. s., 1H), 7.83 (d, J=7.3 Hz, 2H), 7.63-7.78 (m, 5H), 7.39-7.62 (m, 5H), 5.96 (br. s., 1H), 2.15 (br. s., 3H), 1.50 (d, J=2.3 Hz, 5H), 1.20 (d, J=2.0 Hz, 2H).

### Example 7

### 1-(4′-{4-Methyl-5-[(R)-1-(3-trifluoromethyl-phenyl)-ethoxycarbonylamino]-[1,2,3]triazol-1-yl}-biphenyl-4-yl)-cyclopropanecarboxylic acid

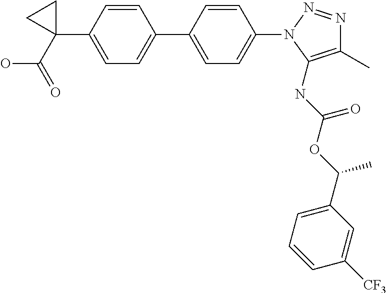 [SMILES: Cc1nnn(-c2ccc(-c3ccc(C4(C(=O)O)CC4)cc3)cc2)c1NC(=O)O[C@H](C)c1cccc(C(F)(F)F)c1]

### Step 1: [3-(4-Bromo-phenyl)-5-methyl-3H-[1,2,3]triazol-4-yl]-carbamic acid (R)-1-(3-trifluoromethyl-phenyl)-ethyl ester

In a 20 mL reaction vial, 3-(4-bromo-phenyl)-5-methyl-3H-[1,2,3]triazole-4-carboxylic acid (195 mg, 0.691 mmol), (R)-1-(3-(trifluoromethyl)phenyl)ethanol (197 mg, 1.04 mmol) and triethylamine (145 mg, 0.2 mL, 1.43 mmol) were combined with toluene (10 mL) to give a yellow solution and to this was added diphenylphosphorylazide (383 mg, 0.3 mL, 1.39 mmol). The vial's atmosphere was purged with nitrogen, sealed, heated in an oil bath at 65° C. for 2.5 h, and cooled to room temperature overnight. The reaction was diluted with EtOAc and washed with water, saturated ammonium chloride, and brine. The aqueous layers were extracted once with EtOAc. The organic layers were combined, dried over MgSO₄, filtered, concentrated, diluted with DCM, and purified by flash chromatography (silica gel, 0% to 30% EtOAc in hexanes). Appropriate fractions combined, concentrated, and dried from DCM/hexanes, to obtain [3-(4-bromo-phenyl)-5-methyl-3H-[1,2,3]triazol-4-yl]-carbamic acid (R)-1-(3-trifluoromethyl-phenyl)-ethyl ester (118.1 mg, 36.4% yield) as a colorless waxy solid. LC/MS calcd. for C₁₉H₁₆BrF₃N₄O₂ (m/e) 468/470, obsd. 469/471 (M+H, ES⁺).

### Step 2: 1-(4′-{4-Methyl-5-[(R)-1-(3-trifluoromethyl-phenyl)-ethoxycarbonylamino]-[1,2,3]triazol-1-yl}-biphenyl-4-yl)-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (89.6 mg, 0.297 mmol), [3-(4-bromo-phenyl)-5-methyl-3H-[1,2,3]triazol-4-yl]-carbamic acid (R)-1-(3-trifluoromethyl-phenyl)-ethyl ester (116 mg, 0.247 mmol), 2-dicyclohexyphosphino-2′,6′-dimethoxybiphenyl (SPhos) (30.4 mg, 0.0742 mmol), tripotassium phosphate (157 mg, 0.742 mmol), and Pd(OAc)₂ (8.3 mg, 0.0371 mmol) were combined with toluene (4 mL) and water (1 mL) (previously purged with nitrogen for 20 min) to give a light yellow suspension. The vial's atmosphere was purged with nitrogen, sealed, heated in oil bath at 80° C. for 3.5 h, and cooled to room temperature overnight. Additional reagents were added 1-[4-(4,4,5,5-tetramethyl-[1,3,2]dioxaborolan-2-yl)-phenyl]-cyclopropanecarboxylic acid methyl ester (45 mg, 0.149 mmol), 2-dicyclohexyphosphino-2′,6′-dimethoxybiphenyl (SPhos) (32 mg, 0.0779 mmol), tripotassium phosphate (57 mg, 0.269 mmol), and Pd(OAc)₂ (10 mg, 0.0445 mmol). The vial's atmosphere was purged with nitrogen, sealed, heated in dry block at 80° C. for 4 h, and cooled to room temperature overnight. The reaction was diluted with EtOAc and washed with water and brine. The aqueous layers were extracted with EtOAc. The organic layers were combined, dried over MgSO₄, filtered, concentrated, dissolved in minimal DCM and purified by flash chromatography (silica gel, 0% to 50% EtOAc in hexanes). Appropriate fractions combined, concentrated, and dried from DCM/hexanes yielding 1-(4′-{4-methyl-5-[(R)-1-(3-trifluoromethyl-phenyl)-ethoxycarbonylamino]-[1,2,3]triazol-1-yl}-biphenyl-4-yl)-cyclopropanecarboxylic acid methyl ester (63.5 mg, 45.5% yield) as a white solid. LC/MS calcd. for C₃₀H₂₇F₃N₄O₄ (m/e) 564, obsd. 565 (M+H, ES⁺).

### Step 3: 1-(4′-{4-Methyl-5-[(R)-1-(3-trifluoromethyl-phenyl)-ethoxycarbonylamino]-[1,2,3]triazol-1-yl}-biphenyl-4-yl)-cyclopropanecarboxylic acid

In a 250 mL round-bottomed flask, 1-(4′-{4-methyl-5-[(R)-1-(3-trifluoromethyl-phenyl)ethoxycarbonylamino]-[1,2,3]triazol-1-yl}-biphenyl-4-yl)-cyclopropanecarboxylic acid methyl ester (214.5 mg, 0.452 mmol) was combined with THF (8 mL) and ethanol (8 mL) to give a yellow solution. To this was dripped in NaOH (1 N, 4.5 mL, 4.5 mmol). The reaction was stirred at room temperature overnight. The reaction was diluted with water, concentrated, diluted with more water and acidified with 1 N HCl. The resulting precipitate was filtered, washed with water and hexanes and dried over house vacuum and in a desiccator yielding 1-(4′-{4-methyl-5-[(R)-1-(3-trifluoromethyl-phenyl)ethoxycarbonylamino]-[1,2,3]triazol-1-yl}-biphenyl-4-yl)-cyclopropanecarboxylic acid (174 mg, 83.6% yield) as a white solid. LC/MS calcd. for C₂₆H₂₈N₄O₄ (m/e) 460, obsd. 461 (M+H, ES⁺). ¹H NMR (DMSO-d₆) δ: 12.39 (br. s., 1H), 9.47 (br. s., 1H), 7.88 (d, J=7.8 Hz, 2H), 7.64 (dd, J=18.7, 8.2 Hz, 4H), 7.46 (d, J=8.3 Hz, 2H), 4.67 (br. s., 1H), 2.36 (br. s., 1H), 2.20 (s, 3H), 1.54-2.02 (m, 6H), 1.43-1.53 (m, 2H), 1.17-1.31 (m, 2H), 1.05 (br. s., 3H).

### Example 8

### 1-{4′-[5-((R)-Indan-1-yloxycarbonylamino)-4-methyl-[1,2,3]triazol-1-yl]-biphenyl-4-yl}-cyclopropanecarboxylic acid

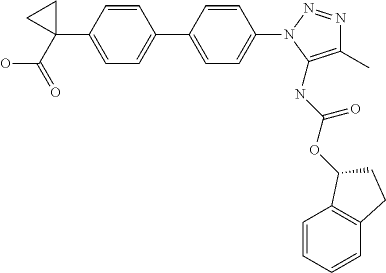 [SMILES: Cc1nnn(-c2ccc(-c3ccc(C4(C(=O)O)CC4)cc3)cc2)c1NC(=O)O[C@@H]1CCc2ccccc21]

### Step 1: [3-(4-Bromo-phenyl)-5-methyl-3H-[1,2,3]triazol-4-yl]-carbamic acid (R)-indan-1-yl ester

In a 20 mL reaction vial, 3-(4-bromo-phenyl)-5-methyl-3H-[1,2,3]triazole-4-carboxylic acid 100 mg, 0.354 mmol), (R)-2,3-dihydro-1H-inden-1-ol (47.6 mg, 0.354 mmol) and triethylamine (35.8 mg, 49.3 μL, 0.354 mmol) were combined with toluene (2.5 mL) to give a yellow suspension and to this was added diphenylphosphorylazide (97.4 mg, 76.3 μL, 0.354 mmol). The vial's atmosphere was purged with nitrogren, sealed, heated in an oil bath at 80° C. for 4 h, and cooled to room temperature overnight. Additional reagents were added, (R)-2,3-dihydro-1H-inden-1-ol (23.8 mg, 0.177 mmol), triethylamine (72.6 mg, 100 μL, 0717 mmol), and diphenylphosphorylazide (97.4 mg, 76.3 μL, 0.354 mmol). The vial's atmosphere was purged with nitrogen, sealed, heated in an oil bath at 80° C. for 2 h, and cooled to room temperature. The reaction was supported on celite and purified by flash chromatography (silica gel, 12 g Redisep, 20 mL/min, 0% to 40% EtOAc in hexanes). Appropriate fractions combined, concentrated, dried from DCM/hexanes, yielding [3-(4-bromo-phenyl)-5-methyl-3H-[1,2,3]triazol-4-yl]-carbamic acid (R)-indan-1-yl ester (81.6 mg, 55.7% yield) as an off-white solid. LC/MS calcd. for C₁₉H₁₇BrN₄O₂ (m/e) 412/414, obsd. 413/415 (M+H, ES⁺).

### Step 2: 1-{4′-[5-((R)-Indan-1-yloxycarbonylamino)-4-methyl-[1,2,3]triazol-1-yl]-biphenyl-4-yl}-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (64.1 mg, 0.212 mmol), [3-(4-bromo-phenyl)-5-methyl-3H-[1,2,3]triazol-4-yl]-carbamic acid (R)-indan-1-yl ester (73 mg, 0.177 mmol), tripotassium phosphate (112 mg, 0.530 mmol), 2-dicyclohexyphosphino-2′,6′-dimethoxybiphenyl (SPhos) (21.8 mg, 0.0530 mmol), and Pd(OAc)₂ (6.0 mg, 0.0265 mmol) were combined with toluene (2 mL) and water (0.5 mL) (previously purged with nitrogen for 20 min) to give a light yellow suspension. The vial's atmosphere was purged with nitrogen, sealed, heated in oil bath at 100° C. for 4 h, and cooled to room temperature overnight. The reaction was diluted with EtOAc (8 mL), filtered through celite, rinsed with EtOAc (2×6 mL), dried, dissolved in minimal DCM and purified by flash chromatography (silica gel, 0% to 50% EtOAc in hexanes). Appropriate fractions combined, concentrated, and dried from DCM/hexanes yielding 1-{4′-[5-((R)-indan-1-yloxycarbonylamino)-4-methyl-[1,2,3]triazol-1-yl]-biphenyl-4-yl}-cyclopropanecarboxylic acid methyl ester (59.5 mg, 66.2% yield) as a white solid. LC/MS calcd. for C₃₀H₂₈N₄O₄ (m/e) 508, obsd. 509 (M+H, ES⁺).

### Step 3: 1-{4′-[5-((R)-Indan-1-yloxycarbonylamino)-4-methyl-[1,2,3]triazol-1-yl]-biphenyl-4-yl}-cyclopropanecarboxylic acid

In a 20 mL round-bottomed flask, 1-{4′-[5-((R)-indan-1-yloxycarbonylamino)-4-methyl-[1,2,3]triazol-1-yl]-biphenyl-4-yl}-cyclopropanecarboxylic acid methyl ester (53 mg, 0.104 mmol) was combined with THF (2 mL) to give a yellow solution. To this was dripped in LiOH (43.8 mg, 1.04 mmol) in water (0.5 mL) heated to partially dissolve. The vial was sealed, heated in an oil bath at 60° C. for 11 h, and cooled to room temperature overnight. The reaction was diluted with water (35 mL) and acidified with 1 N HCl. The resulting precipitate was filtered off and washed with water and hexanes, and dried over house vacuum yielding 1-{4′-[5-((R)-indan-1-yloxycarbonylamino)-4-methyl-[1,2,3]triazol-1-yl]-biphenyl-4-yl}-cyclopropanecarboxylic acid (28 mg, 54.3% yield) as a white solid. LC/MS calcd. for C₂₉H₂₆N₄O₄ (m/e) 494, obsd. 495 (M+H, ES⁺). ¹H NMR (DMSO-d₆) δ: 12.38 (br. s., 1H), 9.56 (br. s., 1H), 7.87 (d, J=8.3 Hz, 2H), 7.68 (d, J=8.3 Hz, 2H), 7.60 (br. s., 2H), 7.48 (d, J=8.0 Hz, 2H), 7.29 (br. s., 3H), 7.11-7.21 (m, 1H), 6.04 (br. s., 1H), 3.00 (br. s., 1H), 2.85 (br. s., 1H), 2.30-2.45 (m, 1H), 2.09-2.28 (m, 3H), 2.00 (br. s., 1H), 1.42-1.61 (m, 2H), 1.21 (d, J=3.0 Hz, 2H).

### Example 9

### 1-{4′-[5-((R)-1,2-Dimethyl-propoxycarbonylamino)-4-methyl-[1,2,3]triazol-1-yl]-biphenyl-4-yl}-cyclopropanecarboxylic acid

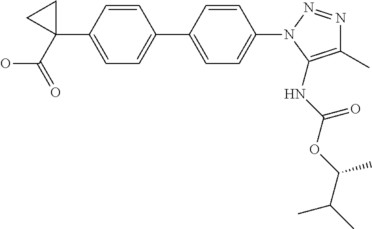 [SMILES: Cc1nnn(-c2ccc(-c3ccc(C4(C(=O)O)CC4)cc3)cc2)c1NC(=O)O[C@H](C)C(C)C]

### Step 1: [3-(4-Bromo-phenyl)-5-methyl-3H-[1,2,3]triazol-4-yl]-carbamic acid (R)-1-2-dimethyl-propyl ester

In a 20 mL reaction vial, 3-(4-bromo-phenyl)-5-methyl-3H-[1,2,3]triazole-4-carboxylic acid 100 mg, 0.354 mmol), (R)-3-methylbutan-2-ol (31.2 mg, 38.6 μL, 0.354 mmol) and triethylamine (35.8 mg, 49.3 μL, 0.354 mmol) were combined with toluene (2.5 mL) to give a yellow suspension and to this was added diphenylphosphorylazide (97.4 mg, 76.3 μL, 0.354 mmol). The vial's atmosphere was purged with nitrogren, sealed, heated in an oil bath at 80° C. for 4 h, and cooled to room temperature overnight. Additional reagents were added, (R)-3-methylbutan-2-ol (15.6 mg, 19.3 μL, 0.177 mmol), triethylamine (72.6 mg, 100 μL, 0717 mmol), and diphenylphosphorylazide (97.4 mg, 76.3 μL, 0.354 mmol). The vial's atmosphere was purged with nitrogen, sealed, heated in an oil bath at 80° C. for 2 h, and cooled to room temperature. The reaction was supported on celite and purified by flash chromatography (silica gel, 0% to 40% EtOAc in hexanes). Appropriate fractions combined, concentrated, dried from DCM/hexanes, yielding [3-(4-bromo-phenyl)-5-methyl-3H[1,2,3]triazol-4-yl]-carbamic acid (R)-1-2-dimethyl-propyl ester (93.6 mg, 71.9% yield) as an off-white solid. LC/MS calcd. for C₁₅H₁₉BrN₄O₂ (m/e) 366/368, obsd. 367/369 (M+H, ES⁺).

### Step 2: 1-{4′-[5-((R)-1,2-Dimethyl-propoxycarbonylamino)-4-methyl-[1,2,3]triazol-1-yl]-biphenyl-4-yl}-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (82.9 mg, 0.274 mmol), [3-(4-bromo-phenyl)-5-methyl-3H-[1,2,3]triazol-4-yl]-carbamic acid (R)-1,2-dimethyl-propyl ester (84 mg, 0.229 mmol), tripotassium phosphate (146 mg, 0.686 mmol), 2-dicyclohexyphosphino-2′,6′-dimethoxybiphenyl (SPhos) (28.2 mg, 0.0686 mmol), and Pd(OAc)₂ (7.7 mg, 0.0343 mmol) were combined with toluene (2 mL) and water (0.5 mL) (previously purged with nitrogen for 20 min) to give a light yellow suspension. The vial's atmosphere was replaced with nitrogen, sealed, heated in oil bath at 100° C. for 4 h, and cooled to room temperature overnight. The reaction was diluted with EtOAc (8 mL), filtered through celite, rinsed with EtOAc (2×6 mL), dried, dissolved in minimal DCM and purified by flash chromatography (silica gel, 0% to 50% EtOAc in hexanes). Appropriate fractions combined, concentrated, and dried from DCM/hexanes yielding 1-{4′-[5-((R)-1,2-dimethyl-propoxycarbonylamino)-4-methyl-[1,2,3]triazol-1-yl]-biphenyl-4-yl}-cyclopropanecarboxylic acid methyl ester (80 mg, 75.6% yield) as a white solid. LC/MS calcd. for C₂₆H₃₀N₄O₄ (m/e) 462, obsd. 463 (M+H, ES⁺).

### Step 3: 1-{4′-[5-((R)-1,2-Dimethyl-propoxycarbonylamino)-4-methyl-[1,2,3]triazol-1-yl]-biphenyl-4-yl}-cyclopropanecarboxylic acid

In a 20 mL round-bottomed flask, 1-{4′-[5-((R)-1,2-dimethyl-propoxycarbonylamino)-4-methyl-[1,2,3]triazol-1-yl]-biphenyl-4-yl}-cyclopropanecarboxylic acid methyl ester (45 mg, 0.0797 mmol) was combined with THF (2 mL) to give a yellow solution. To this was dripped in LiOH (33.5 mg, 0.797 mmol) in water (0.5 mL) heated to partially dissolve. The vial was sealed and heated in an oil bath at 60° C. for 6 h, and cooled to room temperature overnight. The reaction was diluted with water (35 mL) and acidified with 1 N HCl. The resulting precipitate was extracted into the organic layer with EtOAc (2×30 mL), washed with brine (30 mL), dried over MgSO₄, filtered, concentrated, and dried from DCM/hexanes yielding 1-{4′-[5-((R)-1,2-dimethyl-propoxycarbonylamino)-4-methyl-[1,2,3]triazol-1-yl]-biphenyl-4-yl}-cyclopropanecarboxylic acid (42.4 mg, 58.3% yield) as a white solid. LC/MS calcd. for C₂₅H₂₈N₄O₄ (m/e) 448, obsd. (M+H, ES⁺). ¹H NMR (DMSO-d₆) δ: 12.37 (br. s., 1H), 9.43 (br. s., 1H), 7.88 (d, J=8.0 Hz, 2H), 7.57-7.73 (m, 4H), 7.46 (d, J=8.3 Hz, 2H), 4.50 (br. s., 1H), 2.21 (s, 3H), 1.71 (br. s., 1H), 1.40-1.57 (m, 2H), 1.00-1.32 (m, 5H), 0.84 (br. s., 6H).

### Example 10

### 1-{4′-[5-((R)-sec-Butoxycarbonylamino)-4-methyl-[1,2,3]triazol-1-yl]-biphenyl-4-yl}-cyclopropanecarboxylic acid

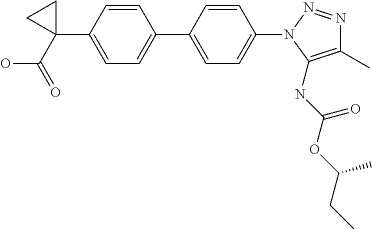 [SMILES: CC[C@@H](C)OC(=O)Nc1c(C)nnn1-c1ccc(-c2ccc(C3(C(=O)O)CC3)cc2)cc1]

### Step 1: [3-(4-Bromo-phenyl)-5-methyl-3H-[1,2,3]triazol-4-yl]-carbamic acid (R)-sec-butyl ester

In a 20 mL reaction vial, 3-(4-bromo-phenyl)-5-methyl-3H-[1,2,3]triazole-4-carboxylic acid 100 mg, 0.354 mmol), (R)-butan-2-ol (26.3 mg, 32.6 μL, 0.354 mmol) and triethylamine (35.8 mg, 49.3 μL, 0.354 mmol) were combined with toluene (2.5 mL) to give a yellow suspension and to this was added diphenylphosphorylazide (97.4 mg, 76.3 μL, 0.354 mmol). The vial's atmosphere was purged with nitrogen, sealed, heated in an oil bath at 80° C. for 4 h, and cooled to room temperature overnight. Additional reagents were added, (R)-butan-2-ol (13.2 mg, 16.3 μL, 0.177 mmol), triethylamine (72.6 mg, 100 μL, 0717 mmol), and diphenylphosphorylazide (97.4 mg, 76.3 μL, 0.354 mmol). The vial's atmosphere was purged with nitrogen, sealed, heated in an oil bath at 80° C. for 2 h, and cooled to room temperature. The reaction was supported on celite and purified by flash chromatography (silica gel, 0% to 40% EtOAc in hexanes). Appropriate fractions combined, concentrated, and dried from DCM/hexanes, yielding [3-(4-bromo-phenyl)-5-methyl-3H-[1,2,3]triazol-4-yl]-carbamic acid (R)-sec-butyl ester (99.5 mg, 79.5% yield) as an off-white solid. LC/MS calcd. for C₁₄H₁₇BrN₄O₂ (m/e) 352/354, obsd. 353/355 (M+H, ES⁺).

### Step 2: 1-{4′-[5-((R)-sec-Butoxycarbonylamino)-4-methyl-[1,2,3]triazol-1-yl]-biphenyl-4-yl}-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (92.4 mg, 0.306 mmol), [3-(4-bromo-phenyl)-5-methyl-3H[1,2,3]triazol-4-yl]-carbamic acid (R)-sec-butyl ester (90 mg, 0.255 mmol), tripotassium phosphate (162 mg, 0.764 mmol), 2-dicyclohexyphosphino-2′,6′-dimethoxybiphenyl (SPhos) (31.4 mg, 0.0764 mmol), and Pd(OAc)₂ (8.6 mg, 0.0382 mmol) were combined with toluene (2 mL) and water (0.5 mL) (previously purged with nitrogen for 20 min) to give a light yellow suspension. The vial's atmosphere was replaced with nitrogen, sealed, heated in oil bath at 100° C. for 4 h, and cooled to room temperature overnight. The reaction was diluted with EtOAc (8 mL), filtered through celite, rinsed with EtOAc (2×6 mL), dried, and dissolved in minimal DCM and purified by flash chromatography (silica gel, 0% to 50% EtOAc in hexanes). Appropriate fractions combined, concentrated, and dried from DCM/hexanes yielding 1-{4′-[5-((R)-sec-butoxycarbonylamino)-4-methyl-[1,2,3]triazol-1-yl]-biphenyl-4-yl}-cyclopropanecarboxylic acid methyl ester (53.6 mg, 46.9% yield) as a white solid. LC/MS calcd. for C₂₅H₂₈N₄O₄ (m/e) 448, obsd. 449 (M+H, ES⁺).

### Step 3: 1-{4′-[5-((R)-sec-Butoxycarbonylamino)-4-methyl-[1,2,3]triazol-1-yl]-biphenyl-4-yl}-cyclopropanecarboxylic acid

In a 20 mL round-bottomed flask, 1-{4′-[5-((R)-sec-butoxycarbonylamino)-4-methyl-[1,2,3]triazol-1-yl]-biphenyl-4-yl}-cyclopropanecarboxylic acid methyl ester (37 mg, 0.0825 mmol) was combined with THF (2 mL) to give a yellow solution. To this was dripped in LiOH (34.6 mg, 0.825 mmol) in water (0.5 mL) heated to partially dissolve. The vial was sealed and heated in an oil bath at 60° C. for 6 h, and cooled to room temperature overnight. The reaction was diluted with water (35 mL) and acidified with 1 N HCl. The resulting precipitate was extracted into the organic layer with EtOAc (2×30 mL), washed with brine (30 mL), dried over MgSO₄, filtered, concentrated, and dried from DCM/hexanes yielding 1-{4′-[5-((R)-sec-butoxycarbonylamino)-4-methyl-[1,2,3]triazol-1-yl]-biphenyl-4-yl}-cyclopropanecarboxylic acid (40 mg, 112% yield) as a white solid. LC/MS calcd for C₂₄H₂₆N₄O₄ (m/e) 434, obsd. 435 (M+H, ES⁺). ¹H NMR (DMSO-d₆) δ: 12.37 (br. s., 1H), 9.44 (br. s., 1H), 7.88 (d, J=8.5 Hz, 2H), 7.56-7.72 (m, 4H), 7.46 (d, J=8.3 Hz, 2H), 4.61 (br. s., 1H), 2.21 (s, 3H), 1.38-1.66 (m, 4H), 1.03-1.34 (m, 5H), 0.85 (dd, J=10.7, 6.9 Hz, 3H).

### Example 11

### 1-[4′-(5-iso-Propoxycarbonylamino-4-methyl-[1,2,3]triazol-1-yl)-biphenyl-4-yl]-cyclopropanecarboxylic acid

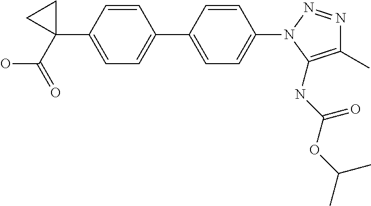 [SMILES: Cc1nnn(-c2ccc(-c3ccc(C4(C(=O)O)CC4)cc3)cc2)c1NC(=O)OC(C)C]

### Step 1: [3-(4-Bromo-phenyl)-5-methyl-3H-[1,2,3]triazol-4-yl]-carbamic acid isopropyl ester

In a 20 mL reaction vial, 3-(4-bromo-phenyl)-5-methyl-3H-[1,2,3]triazole-4-carboxylic acid 100 mg, 0.354 mmol), propan-2-ol (21.3 mg, 27.1 μL, 0.354 mmol) and triethylamine (35.8 mg, 49.3 μL, 0.354 mmol) were combined with toluene (2.5 mL) to give a yellow suspension and to this was added diphenylphosphorylazide (97.4 mg, 76.3 μL, 0.354 mmol). The vial's atmosphere was purged with nitrogren, sealed, heated in an oil bath at 80° C. for 4 h, and cooled to room temperature overnight. Additional reagents were added, propan-2-ol (10.7 mg, 13.6 μL, 0.177 mmol), triethylamine (72.6 mg, 100 μL, 0717 mmol), and diphenylphosphorylazide (97.4 mg, 76.3 μL, 0.354 mmol). The vial's atmosphere was purged with nitrogen, sealed, heated in an oil bath at 80° C. for 2 h, and cooled to room temperature. The reaction was supported on celite and purified by flash chromatography (silica gel, 0% to 40% EtOAc in hexanes). Appropriate fractions combined, concentrated, dried from DCM/hexanes, yielding [3-(4-bromo-phenyl)-5-methyl-3H-[1,2,3]triazol-4-yl]-carbamic acid iso-propyl ester (141 mg, 60% pure, 70.4% yield) as an off-white solid. LC/MS calcd. for C₁₃H₁₅BrN₄O₂ (m/e) 338/340, obsd. 339/341 (M+H, ES⁺).

### Step 2: 1-[4′-(5-iso-Propoxycarbonylamino-4-methyl-[1,2,3]triazol-1-yl)-biphenyl-4-yl]-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (83.4 mg, 0.276 mmol), [3-(4-bromo-phenyl)-5-methyl-3H-[1,2,3]triazol-4-yl]-carbamic acid iso-propyl ester (130 mg, 0.230 mmol), tripotassium phosphate (146 mg, 0.690 mmol), 2-dicyclohexyphosphino-2′,6′-dimethoxybiphenyl (SPhos) (28.3 mg, 0.069 mmol), and Pd(OAc)₂ (7.7 mg, 0.0345 mmol) were combined with toluene (2 mL) and water (0.5 mL) (previously purged with nitrogen for 20 min) to give a light yellow suspension. The vial's atmosphere was replaced with nitrogen, sealed, heated in oil bath at 100° C. for 4 h, and cooled to room temperature overnight. The reaction was diluted with EtOAc (8 mL), filtered through celite, rinsed with EtOAc (2×6 mL), dried, and dissolved in minimal DCM and purified by flash chromatography (silica gel, 0% to 50% EtOAc in hexanes). Appropriate fractions combined, concentrated, and dried from DCM/hexanes yielding 1-[4′-(5-isopropoxycarbonylamino-4-methyl-[1,2,3]triazol-1-yl)-biphenyl-4-yl]-cyclopropanecarboxylic acid methyl ester (44.3 mg, 44.3% yield) as a solid. LC/MS calcd. for C₂₄H₂₆N₄O₄ (m/e) 434, obsd. 435 (M+H, ES⁺).

### Step 3: 1-[4′-(5-iso-Propoxycarbonylamino-4-methyl-[1,2,3]triazol-1-yl)-biphenyl-4-yl]-cyclopropanecarboxylic acid

In a 20 mL round-bottomed flask, 1-[4′-(5-iso-propoxycarbonylamino-4-methyl-[1,2,3]triazol-1-yl)-biphenyl-4-yl]-cyclopropanecarboxylic acid methyl ester (39 mg, 0.0898 mmol) was combined with THF (2 mL) to give a yellow solution. To this was dripped in LiOH (37.7 mg, 0.898 mmol) in water (0.5 mL) heated to partially dissolve. The vial was sealed and heated in an oil bath at 60° C. for 6 h, and cooled to room temperature overnight. The reaction was diluted with water (35 mL) and acidified with 1 N HCl. The resulting precipitate was extracted into the organic layer with EtOAc (2×30 mL), washed with brine (30 mL), dried over MgSO₄, filtered, concentrated, dried from DCM/hexanes yielding 1-[4′-(5-iso-propoxycarbonylamino-4-methyl-[1,2,3]triazol-1-yl)-biphenyl-4-yl]-cyclopropanecarboxylic acid (32.5 mg, 86.1% yield) as a white solid. LC/MS calcd. for C₂₃H₂₄N₄O₄ (m/e) 420, obsd. 421 (M+H+). ¹H NMR (DMSO-d₆) δ: 12.38 (br. s., 1H), 9.43 (br. s., 1H), 7.89 (d, J=8.3 Hz, 2H), 7.57-7.74 (m, 4H), 7.46 (d, J=8.0 Hz, 2H), 4.76 (br. s., 1H), 2.20 (s, 3H), 1.43-1.57 (m, 2H), 1.02-1.34 (m, 8H).

### Example 12

### 1-{4′-[5-(1-Cyclopropyl-ethoxycarbonylamino)-4-methyl-[1,2,3]triazol-1-yl]-biphenyl-4-yl}-cyclopropanecarboxylic acid

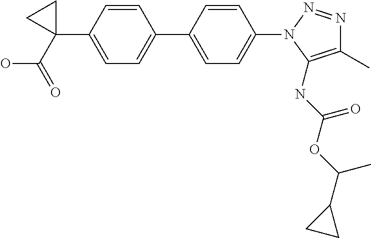 [SMILES: Cc1nnn(-c2ccc(-c3ccc(C4(C(=O)O)CC4)cc3)cc2)c1NC(=O)OC(C)C1CC1]

### Step 1: [3-(4-Bromo-phenyl)-5-methyl-3H-[1,2,3]triazol-4-yl]-carbamic acid 1-cyclopropyl-ethyl ester

In a 20 mL reaction vial, 3-(4-bromo-phenyl)-5-methyl-3H-[1,2,3]triazole-4-carboxylic acid (300 mg, 1.06 mmol), 1-cyclopropylethanol (139 mg, 1.61 mmol) and triethylamine (218 mg, 0.3 mL, 2.15 mmol) were combined with toluene (10 mL) to give a yellow solution and to this was added diphenylphosphorylazide (585 mg, 0.458 mL, 2.13 mmol). The vial's atmosphere was purged with nitrogen, sealed, heated in an oil bath at 65° C. for 2.5 h, and cooled to room temperature overnight. The reaction was diluted with EtOAc and washed with water, saturated ammonium chloride, and brine. The aqueous layers were extracted once with EtOAc. The organic layers were combined, dried over MgSO₄, filtered, concentrated, diluted with DCM, and purified by flash chromatography (silica gel, 0% to 50% EtOAc in hexanes). Appropriate fractions combined, concentrated, and dried from DCM/hexanes, yielding [3-(4-Bromo-phenyl)-5-methyl-3H-[1,2,3]triazol-4-yl]-carbamic acid 1-cyclopropyl-ethyl ester (267 mg, 68.9% yield) as a colorless waxy solid. LC/MS calcd. for C₁₅H₁₇BrN₄O₂ (m/e) 364/366, obsd. 365/367 (M+H, ES⁺).

### Step 2: 1-{4′-[5-(1-Cyclopropyl-ethoxycarbonylamino)-4-methyl-[1,2,3]triazol-1-yl]-biphenyl-4-yl}-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (238 mg, 0.789 mmol), [3-(4-Bromo-phenyl)-5-methyl-3H-[1,2,3]triazol-4-yl]-carbamic acid 1-cyclopropyl-ethyl ester (240 mg, 0.657 mmol), 2-dicyclohexyphosphino-2′,6′-dimethoxybiphenyl (SPhos) (80.9 mg, 0.197 mmol), tripotassium phosphate (418 mg, 1.97 mmol), and Pd(OAc)₂ (22.1 mg, 0.0986 mmol) were combined with toluene (8 mL) and water (2 mL) (previously purged with nitrogen for 20 min) to give a light yellow suspension. The vial's atmosphere was replaced with nitrogen, sealed, heated in dry block at 80° C. for 2.5 h, and cooled to room temperature overnight.

The reaction was diluted with EtOAc and washed with water and brine. The aqueous layers were extracted with EtOAc. The organic layers were combined, dried over MgSO₄, filtered, concentrated, dissolved in minimal DCM and purified by flash chromatography (silica gel, 0% to 50% EtOAc in hexanes). Appropriate fractions combined, concentrated, and dried from DCM/hexanes yielding 1-{4′-[5-(1-cyclopropyl-ethoxycarbonylamino)-4-methyl-[1,2,3]triazol-1-yl]-biphenyl-4-yl}-cyclopropanecarboxylic acid methyl ester (214.2 mg, 70.8% yield) as a white solid. LC/MS calcd. for C₂₆H₂₈N₄O₄ (m/e) 460, obsd. 461 (M+H, ES⁺).

### Step 3: 1-{4′-[5-(1-Cyclopropyl-ethoxycarbonylamino)-4-methyl-[1,2,3]triazol-1-yl]-biphenyl-4-yl}-cyclopropanecarboxylic acid

In a 250 mL round-bottomed flask, 1-{4′-[5-(1-cyclopropyl-ethoxycarbonylamino)-4-methyl-[1,2,3]triazol-1-yl]-biphenyl-4-yl}-cyclopropanecarboxylic acid methyl ester (205 mg, 0.447 mmol) was combined with THF (8 mL) and Ethanol (8 mL) to give a yellow solution. To this was dripped in NaOH (1 N, 4.5 mL, 4.5 mmol). The reaction was stirred at room temperature overnight. The reaction was diluted with water, concentrated, diluted with more water and acidified with 1 N HCl. The resulting precipitate was filtered, washed with water and hexanes and dried over house vacuum and in a desiccator yielding 1-{4′-[5-(1-cyclopropyl-ethoxycarbonylamino)-4-methyl-[1,2,3]triazol-1-yl]-biphenyl-4-yl}-cyclopropanecarboxylic acid (165.4 mg, 82.9% yield) as a white solid. LC/MS calcd. for C₂₅H₂₆N₄O₄ (m/e) 446, obsd. 447 (M+H, ES⁺). ¹H NMR (DMSO-d₆) δ: 12.37 (br. s., 1H), 9.47 (br. s., 1H), 7.88 (d, J=8.3 Hz, 2H), 7.55-7.74 (m, 4H), 7.45 (d, J=8.0 Hz, 2H), 4.12 (br. s., 1H), 2.20 (s, 3H), 1.41-1.55 (m, 2H), 0.84-1.32 (m, 6H), −0.03-0.59 (m, 4H).

### Example 13

### 1-{4′-[5-(1-Cyclobutyl-ethoxycarbonylamino)-4-methyl-[1,2,3]triazol-1-yl]-biphenyl-4-yl}-cyclopropanecarboxylic acid

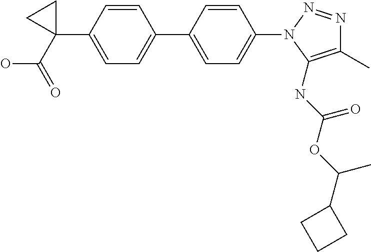 [SMILES: Cc1nnn(-c2ccc(-c3ccc(C4(C(=O)O)CC4)cc3)cc2)c1NC(=O)OC(C)C1CCC1]

### Step 1: [3-(4-Bromo-phenyl)-5-methyl-3H-[1,2,3]triazol-4-yl]-carbamic acid 1-cyclobutyl-ethyl ester

In a 20 mL reaction vial, 3-(4-bromo-phenyl)-5-methyl-3H-[1,2,3]triazole-4-carboxylic acid (300 mg, 1.06 mmol), 1-cyclobutylethanol (170 mg, 1.70 mmol) and triethylamine (218 mg, 0.3 mL, 2.15 mmol) were combined with toluene (10 mL) to give a yellow solution and to this was added diphenylphosphorylazide (585 mg, 0.458 mL, 2.13 mmol). The vial's atmosphere was purged with nitrogen, sealed, heated in an oil bath at 65° C. for 2.5 h, and cooled to room temperature overnight. The reaction was diluted with EtOAc and washed with water, saturated ammonium chloride, and brine. The aqueous layers were extracted once with EtOAc. The organic layers were combined, dried over MgSO₄, filtered, concentrated, diluted with DCM, and purified by flash chromatography (silica gel, 0% to 50% EtOAc in hexanes). Appropriate fractions combined, concentrated, and dried from DCM/hexanes yielding [3-(4-Bromo-phenyl)-5-methyl-3H-[1,2,3]triazol-4-yl]-carbamic acid 1-cyclopropyl-ethyl ester (295.2 mg, 73.2% yield) as a colorless waxy solid. LC/MS calcd. for C₁₆H₁₉BrN₄O₂ (m/e) 378/380, obsd. 379/381 (M+H, ES⁺).

### Step 2: 1-{4′-[5-(1-Cyclobutyl-ethoxycarbonylamino)-4-methyl-[1,2,3]triazol-1-yl]-biphenyl-4-yl}-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (258 mg, 0.854 mmol), [3-(4-Bromo-phenyl)-5-methyl-3H-[1,2,3]triazol-4-yl]-carbamic acid 1-cyclobutyl-ethyl ester (270 mg, 0.712 mmol), 2-dicyclohexyphosphino-2′,6′-dimethoxybiphenyl (SPhos) (87.7 mg, 0.214 mmol), tripotassium phosphate (453 mg, 2.14 mmol), and Pd(OAc)₂ (24.0 mg, 0.107 mmol) were combined with toluene (8 mL) and water (2 mL) (previously purged with nitrogen for 20 min) to give a light yellow suspension. The vial's atmosphere was purged with nitrogen, sealed, heated in dry block at 80° C. for 2.5 h, and cooled to room temperature overnight.

The reaction was diluted with EtOAc and washed with water and brine. The aqueous layers were extracted with EtOAc. The organic layers were combined, dried over MgSO₄, filtered, concentrated, dissolved in minimal DCM and purified by flash chromatography (silica gel, 0% to 50% EtOAc in hexanes). Appropriate fractions combined, concentrated, and dried from DCM/hexanes yielding 1-{4′-[5-(1-cyclobutyl-ethoxycarbonylamino)-4-methyl-[1,2,3]triazol-1-yl]-biphenyl-4-yl}-cyclopropanecarboxylic acid methyl ester (223.6 mg, 66.2% yield) as a white solid. LC/MS calcd. for C₂₇H₃₀N₄O₄ (m/e) 474, obsd. 475 (M+H, ES⁺).

### Step 3: 1-{4′-[5-(1-Cyclobutyl-ethoxycarbonylamino)-4-methyl-[1,2,3]triazol-1-yl]-biphenyl-4-yl}-cyclopropanecarboxylic acid

In a 250 mL round-bottomed flask, 1-{4′-[5-(1-cyclobutyl-ethoxycarbonylamino)-4-methyl-[1,2,3]triazol-1-yl]-biphenyl-4-yl}-cyclopropanecarboxylic acid methyl ester (214.5 mg, 0.452 mmol) was combined with THF (8 mL) and Ethanol (8 mL) to give a yellow solution. To this was dripped in NaOH (1 N, 4.5 mL, 4.5 mmol). The reaction was stirred at room temperature overnight. The reaction was diluted with water, concentrated, diluted with more water and acidified with 1 N HCl. The resulting precipitate was filtered, washed with water and hexanes and dried over house vacuum and in a desiccator yielding 1-{4′-[5-(1-cyclobutyl-ethoxycarbonylamino)-4-methyl-[1,2,3]triazol-1-yl]-biphenyl-4-yl}-cyclopropanecarboxylic acid (174 mg, 83.6% yield) as a white solid. LC/MS calcd. for C₂₆H₂₈N₄O₄ (m/e) 460, obsd. 461 (M+H, ES⁺). ¹H NMR (DMSO-d₆) δ: 12.39 (br. s., 1H), 9.47 (br. s., 1H), 7.88 (d, J=7.8 Hz, 2H), 7.64 (dd, J=18.7, 8.2 Hz, 4H), 7.46 (d, J=8.3 Hz, 2H), 4.67 (br. s., 1H), 2.36 (br. s., 1H), 2.20 (s, 3H), 1.54-2.02 (m, 6H), 1.43-1.53 (m, 2H), 1.17-1.31 (m, 2H), 1.05 (br. s., 3H).

### Example 14

### 1-[4′-(5-tert-Butoxycarbonylamino-4-methyl-[1,2,3]triazol-1-yl)-biphenyl-4-yl]-cyclopropanecarboxylic acid

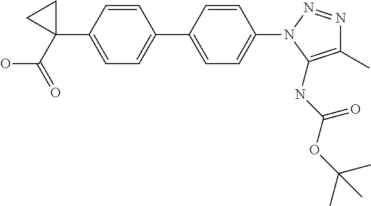 [SMILES: Cc1nnn(-c2ccc(-c3ccc(C4(C(=O)O)CC4)cc3)cc2)c1NC(=O)OC(C)(C)C]

### Step 1: [3-(4-Bromo-phenyl)-5-methyl-3H-[1,2,3]triazol-4-yl]-carbamic acid tert-butyl ester

In a 20 mL reaction vial, 3-(4-bromo-phenyl)-5-methyl-3H-[1,2,3]triazole-4-carboxylic acid (500 mg, 1.77 mmol), 2-methylpropan-2-ol (197 mg, 2.66 mmol) and triethylamine (359 mg, 0.494 mL, 3.54 mmol) were combined with toluene (10 mL) to give a yellow solution and to this was added diphenylphosphorylazide (946 mg, 0.764 mL, 3.54 mmol). The vial's atmosphere was purged with nitrogen, sealed, heated in an oil bath at 65° C. for 2 h, and cooled to room temperature overnight. The reaction was concentrated, diluted with DCM, and purified by flash chromatography (silica gel, 0% to 40% EtOAc in hexanes). Appropriate fractions combined, concentrated, and dried from DCM/hexanes, yielding [3-(4-bromo-phenyl)-5-methyl-3H-[1,2,3]triazol-4-yl]-carbamic acid tert-butyl ester (420 mg, 67.1% yield) as a white solid. LC/MS calcd. for C₁₄H₁₇BrN₄O₂ (m/e) 352/3354, obsd. 353/355 (M+H, ES⁺).

### Step 2: 1-[4′-(5-tert-Butoxycarbonylamino-4-methyl-[1,2,3]triazol-1-yl)-biphenyl-4-yl]-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (395 mg, 1.31 mmol), [3-(4-bromo-phenyl)-5-methyl-3H-[1,2,3]triazol-4-yl]-carbamic acid tert-butyl ester (420 mg, 1.19 mmol), 2-dicyclohexyphosphino-2′,6′-dimethoxybiphenyl (SPhos) (146 mg, 0.357 mmol), tripotassium phosphate (757 mg, 3.57 mmol), and Pd(OAc)₂ (40 mg, 0.178 mmol) were combined with toluene (10 mL) and water (2 mL) (previously purged with nitrogen for 20 min) to give a light yellow suspension. The vial's atmosphere was purged with nitrogen, sealed, heated in dry block at 100° C. for 4 h, and cooled to room temperature overnight. The reaction was filtered, rinsed with water (5 mL) and EtOAc (60 mL). The filtrate was diluted with water (50 mL) and extracted with EtOAc. The aqueous layer was extracted again with EtOAc (40 mL). The organic layers were washed with brine, combined, dried over MgSO₄, filtered, concentrated, dissolved in minimal DCM and purified by flash chromatography (silica gel, 0% to 50% EtOAc in hexanes). Appropriate fractions were combined, concentrated, and dried from DCM/hexanes yielding 1-[4′-(5-tert-butoxycarbonylamino-4-methyl-[1,2,3]triazol-1-yl)-biphenyl-4-yl]-cyclopropanecarboxylic acid methyl ester (420 mg, 78.8% yield) as a white solid. LC/MS calcd. for C₂₅H₂₈N₄O₄ (m/e) 448, obsd. 449 (M+H, ES⁺).

### Step 3: 1-[4′-(5-tert-Butoxycarbonylamino-4-methyl-[1,2,3]triazol-1-yl)-biphenyl-4-yl]-cyclopropanecarboxylic acid

In a 8 mL vial, 1-[4′-(5-tert-butoxycarbonylamino-4-methyl-[1,2,3]triazol-1-yl)-biphenyl-4-yl]-cyclopropanecarboxylic acid methyl ester (22.1 mg, 0.047 mmol) was combined with THF (4 mL) and to this was dripped in NaOH (1 N, 0.5 mL, 0.5 mmol). The reaction was stirred at room temperature for 30 min, water was added (2 mL), and then stirred overnight.

The reaction was diluted with water, concentrated, diluted with more water, and acidified with 1 N HCl. The resulting precipitate was filtered, washed with water and hexanes and dried over house vacuum and in a desiccator yielding 1-[4′-(5-tert-butoxycarbonylamino-4-methyl-[1,2,3]triazol-1-yl)-biphenyl-4-yl]-cyclopropanecarboxylic acid (17.1 mg, 83.7% yield) as a white solid. LC/MS calcd. for C₂₄H₂₆N₄O₄ (m/e) 434, obsd. 435 (M+H, ES⁺). ¹H NMR (DMSO-d₆) δ: 12.40 (br. s., 1H), 9.24 (br. s., 1H), 7.90 (d, J=8.0 Hz, 2H), 7.58-7.71 (m, 4H), 7.46 (d, J=8.3 Hz, 2H), 2.20 (s, 3H), 1.16-1.55 (m, 13H).

### Examples 15

### 1-{3-Fluoro-4′-[4-methyl-5-((R)-1-phenyl-ethoxycarbonylamino)-[1,2,3]triazol-1-yl]-biphenyl-4-yl}-cyclopropanecarboxylic acid

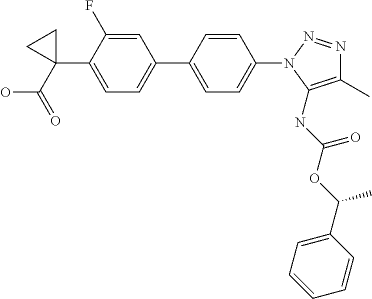 [SMILES: Cc1nnn(-c2ccc(-c3ccc(C4(C(=O)O)CC4)c(F)c3)cc2)c1NC(=O)O[C@H](C)c1ccccc1]

### Step 1: 1-(4-Bromo-2-fluoro-phenyl)-cyclopropanecarbonitrile

In a 0.5 L round-bottomed flask, 2-(4-bromo-2-fluoro-phenyl)acetonitrile (10 g, 46.7 mmol) dissolved in THF (50 mL) was added drop wise under nitrogen to a slurry of NaH (60% dispersion in mineral, 4.11 g, 103 mmol) in DMF (100 mL). The reaction was stirred in an ice bath for 30 min. To this cooled mixture was added 1,2-dibromoethane (8.78 g, 5.71 mL, 46.7 mmol). The reaction was stirred under nitrogen in the ice bath and allowed to warm to room temperature over 3 h. The reaction was diluted with EtOAc (500 mL), filtered, and washed with water and brine. The aqueous layers were extracted with EtOAc (250 mL).

The organic layers were combined, dried over MgSO₄, filtered, and concentrated yielding 1-(4-bromo-2-fluoro-phenyl)cyclopropanecarbonitrile (13.7 g, 122% yield) as a red waxy/solid/oil. LC/MS calcd. for C₁₀H₇BrFN (m/e) 239/241, obsd. 240/242 (M+H, ES⁺).

### Step 2: 1-(4-Bromo-2-fluoro-phenyl)-cyclopropanecarboxylic acid

In a 1 L round-bottomed flask, 1-(4-bromo-2-fluorophenyl)cyclopropanecarbonitrile (11.2 g, 46.7 mmol) and LiOH (58 g, 1.38 mol) were combined with water (230 mL) to give a yellow suspension. The mixture was heated in an oil bath at 100° C. overnight. The mixture was diluted to 1 L with water and ice and extracted with ethyl ether (3×300 mL). There was some white insoluble material between phases that was not included in aqueous layer. The aqueous layer was acidified with concentrated HCl (ca. 110 mL) slowly with addition of ice. A very fine precipitate formed and the milky solution was not filtered but extracted with DCM (4×250 ml). The organic layers were combined, dried over MgSO₄, filtered, and concentrated yielding 1-(4-bromo-2-fluorophenyl)cyclopropanecarboxylic acid (10.87 g, 89.9% yield) as a yellow solid. LC/MS calcd. for C₁₀HsBrFO₂ (m/e) 258/260, obsd. 259/261 (M+H, ES⁺).

### Step 3: 1-(4-Bromo-2-fluoro-phenyl)-cyclopropanecarboxylic acid methyl ester

In a 1 L round-bottomed flask, 1-(4-bromo-2-fluorophenyl)cyclopropanecarboxylic acid (10.8 g, 41.7 mmol) was combined with DMF (180 mL) to give a yellow solution and to this magnetically stirred solution was added K₂CO₃ (17.3 g, 125 mmol). To this was dripped in over 1 h, methyl iodide (47.3 g, 20.9 ml, 333 mmol) dissolved in DMF (20 ml). The yellow suspension was stirred at RT overnight. The reaction was concentrated, diluted with water (500 mL), and extracted with EtOAc (2×500 ml). The EtOAc layers were washed with water brine (250 ml), combined, dried over MgSO₄, filtered, and concentrated yielding 1-(4-bromo-2-fluoro-phenyl)-cyclopropanecarboxylic acid methyl ester (10.3 g, 90.5% yield) as light brown oil. LC/MS calcd. for C₁₁H₁₀BrFO₂ (m/e) 272/274, obsd. 273/275 (M+H, ES⁺).

### Step 4: 1-[2-Fluoro-4-(4,4,5,5-tetramethyl-[1,3,2]dioxaborolan-2-yl)-phenyl]-cyclopropanecarboxylic acid methyl ester

In a 350 mL reaction vial, methyl 1-(4-bromo-2-fluoro-phenyl)-cyclopropanecarboxylate (10.3 g, 37.7 mmol,), BISPIN (11.5 g, 45.3 mmol) and potassium acetate (7.4 g, 75.4 mmol) were combined with 1,4 dioxane (77.2 mL) to give a light brown suspension. The mixture was purged with nitrogen (5 min), PdCl2(DPPF)-DCM (1.54 g, 1.89 mmol) and was added.

The vial was sealed and heated in an oil bath at 80° C. for 4 h. The reaction was filtered through celite, rinsed with DCM, concentrated, diluted with ethyl ether (500 ml), and washed with water (2×500 mL). The first aqueous layer was filtered to remove black solids and rinsed with ethyl ether. This filtrate was combined with the second aqueous layer and extracted with ethyl ether (500 mL). The organic layers were washed with brine (250 mL), combined, dried over MgSO₄, filtered, and concentrated as red oil. The crude material was purified by flash chromatography (silica gel, 0% to 20% EtOAc in hexanes). The appropriate fractions were combined and concentrated yielding the crude product (12.32 g) as a yellow oil.

The crude product was a mixture of starting materials and product and was therefore subjected to the same reaction conditions again. In a 350 mL reaction vial containing the crude product and 1,4 dioxane (200 mL) was added BISPIN (13.6 g, 53.6 mmol) and potassium acetate (8.77 g, 89.3 mmol) to give a light brown suspension. The mixture was purged with nitrogen (5 min), and PdCl₂(DPPF) (3.65 g, 4.47 mmol) was added. The vial was sealed, and the reaction was heated in an oil bath at 80° C. for 3.5 h. The reaction was cooled to room temperature for 5 days. The reaction was diluted with EtOAc and water, concentrated, and diluted with more EtOAc (200 mL) and water (200 ml). The resulting black mixture was inseparable. A partial amount (200 mL) of the aqueous layer (first aqueous layer) was removed from the separatory funnel, and the remaining mixture was washed with brine (2×200 mL, second and third aqueous/brine layers). The black mixture remaining in the separatory funnel was filtered resulting in two phases in the filtrate. This was separated, and the organic layer (first organic layer) was dried over MgSO₄. To the first aqueous and second aqueous/brine layers were added EtOAc (200 mL/each), mixed, filtered through same funnel, separated, and each organic layer was washed with the third aqueous/brine layer. The second and third organic layers were combined with the first containing MgSO₄, dried, filtered, and concentrated yielding the crude product (24 g). The material was dissolved in minimal DCM and purified by flash chromatography (silica gel, 0% to 20% EtOAc in hexanes). The appropriate fractions were concentrated, and dried from DCM/Hex, yielding 1-[2-fluoro-4-(4,4,5,5-tetramethyl-[1,3,2]dioxaborolan-2-yl)-phenyl]-cyclopropanecarboxylic acid methyl ester (6.9 g, 48.2% yield) as an oil which solidifies (crystallizes) as white solid upon cooling to room temperature. LC/MS calcd, for C₁₇H₂₂BFO₄ (m/e) 320, obsd. 321 (M+H, ES⁺).

### Step 5: 1-{3-Fluoro-4′-[4-methyl-5-((R)-1-phenyl-ethoxycarbonylamino)-[1,2,3]triazol-1-yl]-biphenyl-4-yl}-cyclopropanecarboxylic acid methyl ester

In a 20 mL vial, 1-[2-fluoro-4-(4,4,5,5-tetramethyl-[1,3,2]dioxaborolan-2-yl)-phenyl]-cyclopropanecarboxylic acid methyl ester (383 mg, 0.320 mmol), [3-(4-bromo-phenyl)-5-methyl-3H-[1,2,3]triazol-4-yl]-carbamic acid tert-butyl ester (400 mg, 0.997 mmol), 2-dicyclohexyphosphino-2′,6′-dimethoxybiphenyl (SPhos) (123 mg, 0.299 mmol), tripotassium phosphate (635 mg, 2.99 mmol), and Pd(OAc)₂ (33.6 mg, 0.150 mmol) were combined with toluene (10 mL) and water (2 mL) (previously purged with nitrogen for 20 min) to give a light yellow suspension. The vial's atmosphere was purged with nitrogen, sealed, heated in dry block at 80° C. for 4.5 h, and cooled to room temperature overnight. The reaction was diluted with EtOAc, washed with water and brine, dried over MgSO₄, filtered, concentrated, dissolved in minimal DCM and purified by flash chromatography (silica gel, 0% to 50% EtOAc in hexanes). Appropriate fractions combined, concentrated, and dried from DCM/hexanes yielding 1-{3-fluoro-4′-[4-methyl-5-((R)-1-phenyl-ethoxycarbonylamino)[1,2,3]triazol-1-yl]-biphenyl-4-yl}-cyclopropanecarboxylic acid methyl ester (380 mg, 74.1% yield) as a white solid. LC/MS calcd. for C₂₉H₂₇FN₄O₄ (m/e) 514, obsd. 515 (M+H, ES⁺).

### Step 6: 1-{3-Fluoro-4′-[4-methyl-5-((R)-1-phenyl-ethoxycarbonylamino)-[1,2,3]triazol-1-yl]-biphenyl-4-yl}-cyclopropanecarboxylic acid

In a 200 mL round-bottomed flask, 1-{3-fluoro-4′-[4-methyl-5-((R)-1-phenyl-ethoxycarbonylamino)-[1,2,3]triazol-1-yl]-biphenyl-4-yl}-cyclopropanecarboxylic acid methyl ester (380 mg, 0.739 mmol) was combined with THF (10 mL) and MeOH (10 mL) to give a yellow solution. To this was dripped in 1 M NaOH (7.39 mL, 7.39 mmol). The reaction was stirred at room temperature for 1.5 days, stored in a refrigerator for 2.5 days. The reaction was diluted with water, concentrated, diluted with more water and acidified with 1 N HCl. The resulting precipitate was filtered, washed with water and hexanes, and dried over house vacuum and in a desiccator yielding 1-{3-Fluoro-4′-[4-methyl-5-((R)-1-phenyl-ethoxycarbonylamino)-[1,2,3]triazol-1-yl]-biphenyl-4-yl}-cyclopropanecarboxylic acid (303 mg, 82% yield) as a white solid. LC/MS calcd. for C₂₈H₂₅FN₄O₄ (m/e) 500, obsd. 501 (M+H, ES⁺). ¹H NMR (DMSO-d₆) δ: 12.50 (br. s., 1H), 9.70 (br. s., 1H), 7.89 (d, J=6.5 Hz, 2H), 7.43-7.71 (m, 5H), 6.86-7.42 (m, 5H), 5.70 (br. s., 1H), 2.17 (s, 3H), 1.26-1.64 (m, 5H), 1.16-1.25 (m, 2H).

### Examples 16

### 1-{3′-Methoxy-4′-[4-methyl-5-((R)-1-phenyl-ethoxycarbonylamino)-[1,2,3]triazol-1-yl]-biphenyl-4-yl}-cyclopropanecarboxylic acid

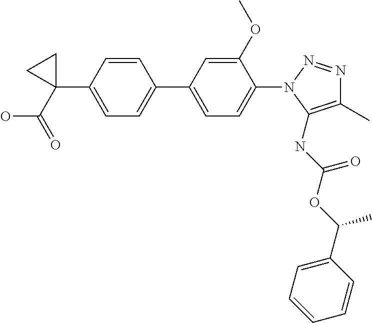 [SMILES: COc1cc(-c2ccc(C3(C(=O)O)CC3)cc2)ccc1-n1nnc(C)c1NC(=O)O[C@H](C)c1ccccc1]

### Step 1: 1-Azido-4-bromo-2-methoxy-benzene

To a mixture of 4-bromo-2-methoxyphenylboronic acid (5 g, 21.7 mmol), sodium azide (2.11 g, 32.5 mmol), and copper(II) acetate (393 mg, 2.17 mmol) in a 100 mL 2-neck RB flask was added methanol (40 mL) at room temperature under nitrogen atmosphere. The resulting brown solution was stirred for 15 h at room temperature and the flask was opened to the air by removing one of the stopper. Within few minutes, it started to change the color to brown suspension and then the stopper was closed again. After 15 h at room temperature, it almost stayed the same brown color. Then, again the stopper was opened, it became slowly darkened. TLC analysis indicated the presence of a new spot. Then, the reaction mixture was heated with heat gun to complete the reaction. During this period, it turned to a light black suspension and after 1 h at ambient temperature the reaction mixture was poured into a mixture of saturated ammonium chloride and ammonium hydroxide. The organic compound was extracted into diethyl ether (2×100 mL) and the combined extracts were washed with brine solution and dried over anhydrous MgSO₄. Filtration and concentration gave the crude oil which was purified using an ISCO (120 g) column chromatography eluting with hexanes. The fractions were combined and the solvent was removed under vacuum to obtain 1-azido-4-bromo-2-methoxy-benzene a light yellow oil (4.14 g, 84% yield).

### Step 2: 3-(4-Bromo-2-methoxy-phenyl)-5-methyl-3H-[1,2,3]triazole-4-carboxylic acid methyl ester

In a solution of 1-azido-4-bromo-2-methoxy-benzene (3.95 g, 17.3 mmol) and methyl but-2-ynoate (1.7 g, 17.3 mmol) in toluene (36 mL) was heated to 150° C. and stirred for 15 h at this temperature at which time TLC analysis indicated the presence of two new spots. During the 15 h stirring, it was slowly turned from a light yellow color solution to the dark brown solution. Then, the heating was stopped and the toluene was removed under vacuum to obtain the dark brown oil (˜8.0 g) which was purified using an ISCO (120 g) column chromatography eluting with 0-50% EA in hexanes to obtain all the spots. The desired regioisomer 3-(4-bromo-2-methoxy-phenyl)-5-methyl-3H-[1,2,3]triazole-4-carboxylic acid methyl ester was isolated as dark brown viscous oil (250 mg, 4.5% yield). LC/MS calcd. for C₁₂H₁₂BrN₃O₃ (m/e) 326, obsd. 328 [M+H, ES⁺].

### Step 3: 3-(4-Bromo-2-methoxy-phenyl)-5-methyl-3H-[1,2,3]triazole-4-carboxylic acid

To a solution of methyl 1-(4-bromo-2-methoxy-phenyl)-4-methyl-1H-1,2,3-triazole-5-carboxylate (220 mg, 0.68 mmol) in THF (4 mL) was added an excess of lithium hydroxide monohydrate (283 mg, 6.75 mmol) in water (1.0 mL) at room temperature. The resulting brown solution was heated to 50° C. in an oil bath for 3 h at which time LCMS analysis indicated the absence of starting material. Then, it was cooled to room temperature and the solvent was removed under vacuum. After dilution with NaOH (˜5 mL) and water (50 mL), the neutral impurities were extracted into diethyl ether (2×50 mL). The basic aqueous layer was neutralized with 1 N HCl and the resulting solids were collected by filtration and washed with water and hexanes. After air drying, 155 mg (74% yield) of 3-(4-bromo-2-methoxy-phenyl)-5-methyl-3H-[1,2,3]triazole-4-carboxylic acid was isolated as off-white solid. LC/MS calcd. for C₁₁H₁₀BrN₃O₃ (m/e) 312, obsd. 314.0 [M+H, ES⁺].

### Step 4: [3-(4-Bromo-2-methoxy-phenyl)-5-methyl-3H-[1,2,3]triazol-4-yl]-carbamic acid (R)-1-phenyl-ethyl ester

To a suspension of 1-(4-bromo-2-methoxy-phenyl)-4-methyl-1H-1,2,3-triazole-5-carboxylic acid (152 mg, 0.49 mmol) in toluene (4 mL) in a vial was added triethylamine (49.3 mg, 67.9 μL, 0.489 mmol) at room temperature under nitrogen atmosphere. To the resulting brown solution were added diphenylphosphorylazide (134 mg, 105 μL, 0.49 mmol) followed by (R)-1-phenylethanol (59.5 mg, 58.8 μL, 0.49 mmol) at room temperature under nitrogen atmosphere. Then, the rubber septum was replaced with a cap and the brown solution was heated to 80° C. and it was stirred for 3 h at this temperature. Then, the reaction mixture was cooled to room temperature and the solvent was removed under vacuum. The brown oil was purified using an ISCO (80 g) column chromatography eluting with 0-100% EA in hexanes. The desired fractions were combined and the solvent was removed under vacuum to obtain [3-(4-bromo-2-methoxy-phenyl)-5-methyl-3H-[1,2,3]triazol-4-yl]-carbamic acid (R)-1-phenyl-ethyl ester (173 mg, 82% yield) as a white solid. LC/MS calcd. for C₁₉H₁₉BrN₄O₃ (m/e) 431, obsd. 432.9 [M+H, ES⁺].

### Step 5: 1-{3′-Methoxy-4′-[4-methyl-5-((R)-1-phenyl-ethoxycarbonylamino)[1,2,3]triazol-1-yl]-biphenyl-4-yl}-cyclopropanecarboxylic acid methyl ester

To a mixture of [3-(4-bromo-2-methoxy-phenyl)-5-methyl-3H-[1,2,3]triazol-4-yl]-carbamic acid (R)-1-phenyl-ethyl ester (100 mg, 0.23 mmol), methyl 1-(4-(4,4,5,5-tetramethyl-1,3,2-dioxaborolan-2-yl)phenyl)cyclopropanecarboxylate (105 mg, 0.35 mmol), palladium(II) acetate (7.81 mg, 0.035 mmol), 2-dicyclohexylphosphino-2′,6′-dimethoxybiphenyl (28.6 mg, 0.69 mmol), and potassium phosphate tribasic (148 mg, 0.69 mmol) were added previously degassed toluene (4.5 mL) and water (1.0 mL) at room temperature under nitrogen atmosphere. The resulting light yellow suspension was heated to 105° C. and stirred for 2 h by which time TLC analysis indicated the absence of starting material. Within 1 h, the reaction mixture was converted to a black reaction mixture. After 2 h, the reaction mixture was cooled to room temperature and poured into a mixture of water and brine solution. The organic compound was extracted into EA (2×50 mL) and the combined extracts were washed with brine solution and dried over anhydrous MgSO₄. Filtration and concentration gave the crude residue which was purified by using an ISCO (40 g) column chromatography eluting with 0-100% EA in hexanes. The desired fractions were combined and the solvent was removed under vacuum to isolate 1-{3′-methoxy-4′-[4-methyl-5-((R)-1-phenyl-ethoxycarbonylamino)-[1,2,3]triazol-1-yl]-biphenyl-4-yl}-cyclopropanecarboxylic acid methyl ester (95 mg, 75% yield) as off-white solid. LC/MS calcd. for C₃₀H₃₀N₄O₅ (m/e) 526, obsd. 527.1 [M+H, ES⁺].

### Step 6: 1-{3′-Methoxy-4′-[4-methyl-5-((R)-1-phenyl-ethoxycarbonylamino)[1,2,3]triazol-1-yl]-biphenyl-4-yl}-cyclopropanecarboxylic acid

To a solution of 1-{2′-methoxy-4′-[4-methyl-5-((R)-1-phenyl-ethoxycarbonylamino)[1,2,3]triazol-1-yl]-biphenyl-4-yl}-cyclopropanecarboxylic acid methyl ester (87 mg, 0.17 mmol) in THF (4.5 mL) and ethanol (4.5 mL) was added an excess of 1 M sodium hydroxide (1.65 mL, 1.65 mmol) at room temperature. The resulting colorless solution was stirred for 15 h at which time LCMS analysis indicated the absence of starting material. Then, the solvent was removed under vacuum and the basic aqueous layer was diluted with water and neutralized with 1 N HCl. The resulting solids were collected by filtration and washed with water and hexanes. After air drying, 50 mg (59% yield) of 1-{3′-methoxy-4′-[4-methyl-5-((R)-1-phenyl-ethoxycarbonylamino)-[1,2,3]triazol-1-yl]-biphenyl-4-yl}-cyclopropanecarboxylic acid was isolated as a white solid. LC/MS calcd. for C₂₉H₂₈N₄O₅ (m/e) 512, obsd. 513.1 [M+H, ES⁺]. ¹H NMR (DMSO-d₆) δ: 12.40 (br. s., 1H), 9.43 (br. s., 1H), 7.70 (d, J=8.3 Hz, 2H), 7.41-7.51 (m, 3H), 7.18-7.39 (m, 7H), 5.70 (d, J=6.3 Hz, 1H), 3.74 (s, 3H), 2.16 (s, 3H), 1.34-1.56 (m, 5H), 1.16-1.24 (m, 2H).

### Example 17

### 1-{4′-[4-Ethyl-5-((R)-1-phenyl-ethoxycarbonylamino)-[1,2,3]triazol-1-yl]-biphenyl-4-yl}-cyclopropanecarboxylic acid

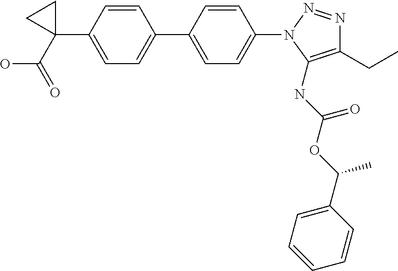 [SMILES: CCc1nnn(-c2ccc(-c3ccc(C4(C(=O)O)CC4)cc3)cc2)c1NC(=O)O[C@H](C)c1ccccc1]

### Step 1: 3-(4-Bromo-phenyl)-5-ethyl-3H-[1,2,3]triazole-4-carboxylic acid ethyl ester

To a solution of 1-azido-4-bromobenzene (5 g, 25.2 mmol) in toluene (50 mL) was added a neat 2-pentynoic acid ethyl ester (3.19 g, 25.2 mmol) in a 250 mL sealed tube and then it was kept for 2 minutes under nitrogen atmosphere. Then, the flask was sealed with a tight cap and the resulting light yellow solution was heated to 150° C. and stirred for 2 h at which time TLC analysis indicated the presence of two new spots and LCMS analysis indicated the presence of the desired mass. Then, the dark brown reaction mixture was cooled to room temperature and the solvent was removed under vacuum. The resulting dark brown residue (8.3 g) was purified using an ISCO (330 g) column chromatography eluting with 0-50% EA in hexanes. The top spot in TLC was isolated as a desired 3-(4-bromo-phenyl)-5-ethyl-3H[1,2,3]triazole-4-carboxylic acid ethyl ester as off-white solid (2.83 g, 34.6% yield) and the bottom spot was confirmed as a wrong regioisomer, 3-(4-bromo-phenyl)-5-ethyl-3H[1,2,3]triazole-4-carboxylic acid ethyl ester which was isolated as a light brown oil (3.44 g, 42% yield). LC/MS calcd. for C₁₃H₁₄BrN₃O₂ (m/e) 324, obsd. 326 [M+H, ES⁺].

### Step 2: 3-(4-Bromo-phenyl)-5-ethyl-3H-[1,2,3]triazole-4-carboxylic acid

To a brown solution of 3-(4-bromo-phenyl)-5-ethyl-3H-[1,2,3]triazole-4-carboxylic acid ethyl ester (2.8 g, 8.64 mmol) in THF (40 mL) was added a solution of lithium hydroxide monohydrate (1.81 g, 43.2 mmol) in water (10 mL) at room temperature. The resulting brown solution was stirred for 15 h at room temperature at which time LCMS analysis indicated the absence of starting material. Then, the solvent was removed under vacuum. After dilution with NaOH (˜5 mL) and water (50 mL), the neutral impurities were extracted into diethyl ether (100 mL) and it also removed the brown color. The basic aqueous layer was neutralized with 1 N HCl and the resulting white solids were collected by filtration and washed with water and hexanes. After air drying, 2.13 g (83% yield) of 3-(4-bromo-phenyl)-5-ethyl-3H-[1,2,3]triazole-4-carboxylic acid was isolated as off-white solid. LC/MS calcd. for C₁₁H₁₀BrN₃O₂ (m/e) 296, obsd. 297.7 [M+H, ES⁺].

### Step 3: [3-(4-Bromo-phenyl)-5-ethyl-3H-[1,2,3]triazol-4-yl]-carbamic acid (R)-1-phenyl-ethyl ester

To a suspension of 3-(4-bromo-phenyl)-5-ethyl-3H-[1,2,3]triazole-4-carboxylic acid (592 mg, 2.0 mmol) in toluene (10 mL) in a vial was added triethylamine (202 mg, 279 μL, 2.0 mmol) at room temperature under nitrogen atmosphere. To the resulting brown solution were added diphenylphosphorylazide (550 mg, 431 μL, 2.0 mmol) followed by (R)-1-phenylethanol (244 mg, 241 μL, 2.0 mmol) at room temperature under nitrogen atmosphere. Then, the resulting light brown solution was heated to 80° C. and stirred for 2 h at this temperature. Then, the clear light brown reaction mixture was cooled to room temperature and the solvent was removed under vacuum. The brown oil was purified using an ISCO (80 g) column chromatography eluting with 0-100% EA in hexanes to obtain the desired [3-(4-bromo-phenyl)-5-ethyl-3H-[1,2,3]triazol-4-yl]-carbamic acid (R)-1-phenyl-ethyl ester (696 mg, 89% yield) as a white solid. LC/MS calcd. for C₁₉H₁₉BrN₄O₂ (m/e) 415, obsd. 417 [M+H, ES⁺].

### Step 4: 1-{4′-[4-Ethyl-5-((R)-1-phenyl-ethoxycarbonylamino)-[1,2,3]triazol-1-yl]-biphenyl-4-yl}-cyclopropanecarboxylic acid methyl ester

To a mixture of [3-(4-bromo-phenyl)-5-ethyl-3H-[1,2,3]triazol-4-yl]-carbamic acid (R)-1-phenyl-ethyl ester (300 mg, 0.72 mmol), methyl 1-(4-(4,4,5,5-tetramethyl-1,3,2-dioxaborolan-2-yl)phenyl)cyclopropanecarboxylate (327 mg, 1.08 mmol), palladium(II) acetate (24.3 mg, 0.11 mmol), 2-dicyclohexylphosphino-2′,6′-dimethoxybiphenyl (89.0 mg, 0.22 mmol), and potassium phosphate tribasic (460 mg, 2.17 mmol) were added previously degassed toluene (9.0 mL) and water (2.0 mL) at room temperature under nitrogen atmosphere. The resulting light yellow suspension was heated to 105° C. and stirred for 2 h by which time TLC analysis indicated the absence of starting material. Then, the reaction mixture was cooled to room temperature and poured into a mixture of water and brine solution. The organic compound was extracted into EA (2×50 mL) and the combined extracts were washed with brine solution and dried over anhydrous MgSO₄. Filtration and concentration gave the crude residue which was purified by using an ISCO (80 g) column chromatography eluting with 0-100% EA in hexanes. The desired fractions were combined and the solvent was removed under vacuum to isolate the desired 1-{4′-[4-ethyl-5-((R)-1-phenyl-ethoxycarbonylamino)-[1,2,3]triazol-1-yl]-biphenyl-4-yl}-cyclopropanecarboxylic acid methyl ester (240 mg, 65% yield). LC/MS calcd. for C₃₀H₃₀N₄O₄ (m/e) 510, obsd. 511.1 [M+H, ES⁺].

### Step 5: 1-{4′-[4-Ethyl-5-((R)-1-phenyl-ethoxycarbonylamino)-[1,2,3]triazol-1-yl]-biphenyl-4-yl}-cyclopropanecarboxylic acid

To a solution of 1-{4′-[4-ethyl-5-((R)-1-phenyl-ethoxycarbonylamino)-[1,2,3]triazol-1-yl]-biphenyl-4-yl}-cyclopropanecarboxylic acid methyl ester (234 mg, 0.46 mmol) in THF (10 mL) and ethanol (10 mL) was added an excess of 1.0 M sodium hydroxide (4.58 mL, 4.58 mmol) solution at room temperature. The resulting colorless solution was stirred for 15 h at which time LCMS analysis indicated the absence of starting material. Then, the solvent was removed under vacuum and the basic aqueous layer was diluted with water and neutralized with 1 N HCl. The resulting solids were collected by filtration and washed with water and hexanes. After air drying, 193 mg (85% yield) of 1-{4′-[4-ethyl-5-((R)-1-phenyl-ethoxycarbonylamino)-[1,2,3]triazol-1-yl]-biphenyl-4-yl}-cyclopropanecarboxylic acid was isolated as a white solid. LC/MS calcd. for C₂₉H₂₈N₄O₄ (m/e) 496, obsd. 497.1 [M+H, ES⁺]. ¹H NMR (DMSO-d₆) δ: 12.40 (br. s., 1H), 9.66 (br. s., 1H), 7.83 (d, J=6.5 Hz, 2H), 7.67 (d, J=8.0 Hz, 2H), 7.58 (d, J=7.3 Hz, 2H), 7.47 (d, J=8.0 Hz, 2H), 7.34 (br. s., 5H), 5.71 (br. s., 1H), 2.56 (d, J=7.5 Hz, 2H), 1.36-1.60 (m, 5H), 1.16-1.23 (m, 5H).

### Examples 18

### {4′-[4-Ethyl-5-((R)-1-phenyl-ethoxycarbonylamino)-[1,2,3]triazol-1-yl]-biphenyl-4-yl}-acetic acid

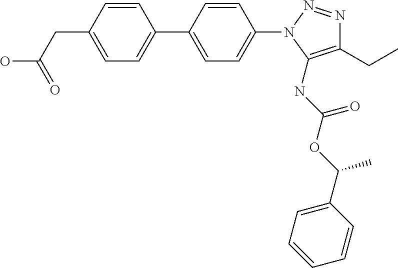 [SMILES: CCc1nnn(-c2ccc(-c3ccc(CC(=O)O)cc3)cc2)c1NC(=O)O[C@H](C)c1ccccc1]

### Step 1: {4′-[4-Ethyl-5-((R)-1-phenyl-ethoxycarbonylamino)-[1,2,3]triazol-1-yl]-biphenyl-4-yl}-acetic acid ethyl ester

To a mixture of (R)-1-phenylethyl 1-(4-bromophenyl)-4-ethyl-1H-1,2,3-triazol-5-ylcarbamate (200 mg, 0.48 mmol), ethyl 2-(4-(4,4,5,5-tetramethyl-1,3,2-dioxaborolan-2-yl)phenyl)acetate (210 mg, 0.72 mmol), palladium(II) acetate (16.2 mg, 0.072 mmol), 2-dicyclohexylphosphino-2′,6′-dimethoxybiphenyl (59.3 mg, 0.144 mmol), and potassium phosphate tribasic (307 mg, 1.44 mmol) were added previously degassed toluene (4.5 mL) and water (1.0 mL) at room temperature under nitrogen atmosphere. The resulting light yellow suspension was heated to 105° C. and stirred for 1 h by which time TLC analysis indicated the absence of starting material. Then, the reaction mixture was cooled to room temperature and poured into a mixture of water and brine solution. The organic compound was extracted into EA (2×50 mL) and the combined extracts were washed with brine solution and dried over anhydrous MgSO₄. Filtration and concentration gave the crude residue which was purified by using an ISCO (80 g) column chromatography eluting with 0-100% EA in hexanes. The desired fractions were combined and the solvent was removed under vacuum to isolate {4′-[4-ethyl-5-((R)-1-phenyl-ethoxycarbonylamino)-[1,2,3]triazol-1-yl]-biphenyl-4-yl}-acetic acid ethyl ester (133 mg, 55% yield). LC/MS calcd. for C₂₈H₂₈N₄O₄ (m/e) 498, obsd. 499.1 [M+H, ES⁺].

### Step 2: {4′-[4-Ethyl-5-((R)-1-phenyl-ethoxycarbonylamino)-[1,2,3]triazol-1-yl]-biphenyl-4-yl}-acetic acid

To a solution of {4′-[4-ethyl-5-((R)-1-phenyl-ethoxycarbonylamino)-[1,2,3]triazol-1-yl]-biphenyl-4-yl}-acetic acid ethyl ester (105 mg, 0.21 mmol) in THF (5 mL) and ethanol (5.0 mL) was added an excess of 1 M sodium hydroxide (2.11 mL, 2.11 mmol) solution in water at room temperature. The resulting colorless solution was stirred for 15 h at room temperature by which time LCMS analysis indicated the absence of starting material. Then, the solvent was removed under vacuum and the basic aqueous solution was neutralized with 1 N HCl. The resulting solids were collected by filtration and washed with water and hexanes. After air drying, 74 mg (75% yield) of {4′-[4-ethyl-5-((R)-1-phenyl-ethoxycarbonylamino)[1,2,3]triazol-1-yl]-biphenyl-4-yl}-acetic acid was isolated as a white solid. LC/MS calcd. for C₂₇H₂₆N₄O₄ (m/e) 470, obsd. 470.1 [M+H, ES⁺]. ¹H NMR (DMSO-d₆) δ: 12.43 (br. s., 1H), 9.16-9.88 (m, 1H), 7.84 (d, J=6.0 Hz, 2H), 7.70 (d, J=8.0 Hz, 2H), 7.52-7.64 (m, 2H), 7.42 (d, J=8.0 Hz, 2H), 7.07-7.38 (m, 5H), 5.70 (br. s., 1H), 3.66 (s, 2H), 2.57 (d, J=7.0 Hz, 2H), 1.48 (br. s., 3H), 1.20 (t, J=7.5 Hz, 3H).

### Example 19

### 1-(4′-{4-Ethyl-5-[(R)-1-(3-trifluoromethyl-phenyl)-ethoxycarbonylamino]-[1,2,3]triazol-1-yl}-biphenyl-4-yl)-cyclopropanecarboxylic acid

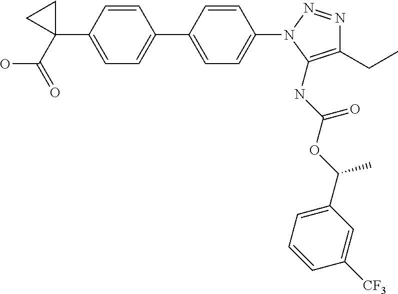 [SMILES: CCc1nnn(-c2ccc(-c3ccc(C4(C(=O)O)CC4)cc3)cc2)c1NC(=O)O[C@H](C)c1cccc(C(F)(F)F)c1]

### Step 1: [3-(4-Bromo-phenyl)-5-ethyl-3H-[1,2,3]triazol-4-yl]-carbamic acid (R)-1-(3-trifluoromethyl-phenyl)-ethyl ester

To a suspension of 1-(4-bromophenyl)-4-ethyl-1H-1,2,3-triazole-5-carboxylic acid (623 mg, 2.1 mmol) in toluene (10 mL) in a vial was added triethylamine (213 mg, 293 μL, 2.1 mmol) at room temperature under nitrogen atmosphere. To the resulting brown solution were added diphenylphosphorylazide (579 mg, 453 μL, 2.1 mmol) and (R)-1-(3-(trifluoromethyl)phenyl)ethanol (400 mg, 2.1 mmol) at room temperature under nitrogen atmosphere. Then, the resulting light brown solution was heated to 80° C. and stirred for 2.5 h at this temperature. Then, the clear light brown reaction mixture was cooled to room temperature and the solvent was removed under vacuum. The resulting brown oil was purified using an ISCO (80 g) column chromatography eluting with 0-100% EA in hexanes. The desired fractions were combined and the solvent was removed under vacuum to obtain [3-(4-bromo-phenyl)-5-ethyl-3H-[1,2,3]triazol-4-yl]-carbamic acid (R)-1-(3-trifluoromethyl-phenyl)-ethyl ester (735 mg, 72% yield) as a white solid. LC/MS calcd. for C₂₀H₁₈BrF₃N₄O₂ (m/e) 483, obsd. 484.9 [M+H, ES⁺].

### Step 2: 1-(4′-{4-Ethyl-5-[(R)-1-(3-trifluoromethyl-phenyl)-ethoxycarbonylamino]-[1,2,3]triazol-1-yl}-biphenyl-4-yl)-cyclopropanecarboxylic acid methyl ester

To a mixture of [3-(4-bromo-phenyl)-5-ethyl-3H-[1,2,3]triazol-4-yl]-carbamic acid (R)-1-(3-trifluoromethyl-phenyl)-ethyl ester (300 mg, 0.62 mmol), methyl 1-(4-(4,4,5,5-tetramethyl-1,3,2-dioxaborolan-2-yl)phenyl)cyclopropanecarboxylate (281 mg, 0.93 mmol), palladium(II) acetate (20.9 mg, 0.09 mmol), 2-dicyclohexylphosphino-2′,6′-dimethoxybiphenyl (76.5 mg, 0.19 mmol), and potassium phosphate tribasic (395 mg, 1.86 mmol) in a vial were added freshly degassed toluene (4.5 mL) and water (1.0 mL) at room temperature under nitrogen atmosphere. Then, the cap was closed and the resulting light yellow suspension was heated to 105° C. and stirred for 1 h by which time TLC analysis indicated the absence of starting material. Then, the reaction mixture was cooled to room temperature and poured into a mixture of water and brine solution. The organic compound was extracted into EA (2×50 mL) and the combined extracts were washed with brine solution and dried over anhydrous MgSO₄. Filtration and concentration gave the crude residue which was purified by using an ISCO (80 g) column chromatography eluting with 0-100% EA in hexanes. The desired fractions were combined and the solvent was removed under vacuum to isolate 1-(4′-{4-ethyl-5-[(R)-1-(3-trifluoromethyl-phenyl)-ethoxycarbonylamino]-[1,2,3]triazol-1-yl}-biphenyl-4-yl)-cyclopropanecarboxylic acid methyl ester (245 mg, 68% yield). LC/MS calcd. for C₃₁H₂₉F₃N₄O₄ (m/e) 578, obsd. 579.4 [M+H, ES⁺].

### Step 3: 1-(4′-{4-Ethyl-5-[(R)-1-(3-trifluoromethyl-phenyl)-ethoxycarbonylamino]-[1,2,3]triazol-1-yl}-biphenyl-4-yl)-cyclopropanecarboxylic acid

To a solution of 1-(4′-{4-ethyl-5-[(R)-1-(3-trifluoromethyl-phenyl)-ethoxycarbonylamino]-[1,2,3]triazol-1-yl}-biphenyl-4-yl)-cyclopropanecarboxylic acid methyl ester (240 mg, 0.42 mmol) in THF (5 mL) and ethanol (5 mL) was added an excess of 1.0 N sodium hydroxide (4.15 mL, 4.15 mmol) solution in water at room temperature. The resulting solution was stirred for 15 h at room temperature at which time TLC analysis indicated the absence of starting material. Then, it was diluted with water and the solvent was removed under vacuum. The neutral impurities were extracted into diethyl ether (100 mL) and the basic aqueous layer was neutralized with 1.0 N HCl. The resulting precipitate was extracted into EA (2×45 mL) and the combined extracts were washed with brine solution. After drying and filtration, the solvent was removed under vacuum to obtain 1-(4′-{4-ethyl-5-[(R)-1-(3-trifluoromethyl-phenyl)-ethoxycarbonylamino]-[1,2,3]triazol-1-yl}-biphenyl-4-yl)-cyclopropanecarboxylic acid (219 mg, 93.5% yield). LC/MS calcd. for C₃₀H₂₇F₃N₄O₄ (m/e) 564, obsd. 565.3 [M+H, ES⁺]. ¹H NMR (DMSO-d₆) δ: 12.24 (br. s., 1H), 9.28-9.93 (m, 1H), 7.82 (d, J=6.8 Hz, 2H), 7.53-7.76 (m, 7H), 7.47 (d, J=8.3 Hz, 3H), 5.80 (br. s., 1H), 2.54-2.64 (m, 2H), 1.37-1.61 (m, 5H), 1.13-1.23 (m, 5H).

### Examples 20

### {4′-[4-Ethyl-5-((R)-1-(3-trifluoromethyl-phenyl-ethoxycarbonylamino)-[1,2,3]triazol-1-yl]-biphenyl-4-yl}-acetic acid

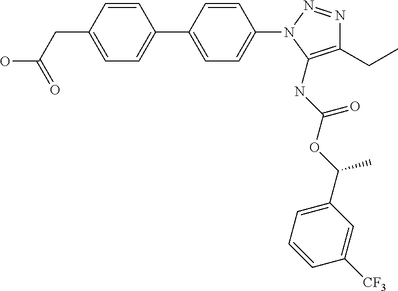 [SMILES: CCc1nnn(-c2ccc(-c3ccc(CC(=O)O)cc3)cc2)c1NC(=O)O[C@H](C)c1cccc(C(F)(F)F)c1]

### Step 1: {4′-[4-Ethyl-5-((R)-1-(3-trifluoromethyl-phenyl-ethoxycarbonylamino)[1,2,3]triazol-1-yl]-biphenyl-4-yl}-acetic acid ethyl ester

To a mixture of (R)-1-(3-(trifluoromethyl)phenyl)ethyl 1-(4-bromophenyl)-4-ethyl-1H-1,2,3-triazol-5-ylcarbamate (200 mg, 0.41 mmol), ethyl 2-(4-(4,4,5,5-tetramethyl-1,3,2-dioxaborolan-2-yl)phenyl)acetate (120 mg, 0.41 mmol), palladium(II) acetate (13.9 mg, 0.06 mmol), 2-dicyclohexylphosphino-2′,6′-dimethoxybiphenyl (51.0 mg, 0.12 mmol), and potassium phosphate tribasic (264 mg, 1.24 mmol) in a vial were added freshly degassed toluene (4.5 mL) and water (1.0 mL) at room temperature under nitrogen atmosphere. Then, the cap was closed and the resulting light yellow suspension was heated to 105° C. and stirred for 1 h by which time TLC analysis indicated the absence of starting material. Then, the reaction mixture was cooled to room temperature and poured into a mixture of water and brine solution. The organic compound was extracted into EA (2×50 mL) and the combined extracts were washed with brine solution and dried over anhydrous MgSO₄. Filtration and concentration gave the crude residue which was purified by using an ISCO (80 g) column chromatography eluting with 0-100% EA in hexanes. The desired fractions were combined and the solvent was removed under vacuum to isolate {4′-[4-ethyl-5-((R)-1-(3-trifluoromethyl-phenyl-ethoxycarbonylamino)-[1,2,3]triazol-1-yl]-biphenyl-4-yl}-acetic acid ethyl ester (194 mg, 83% yield). LC/MS calcd. for C₃₀H₂₉F₃N₄O₄ (m/e) 566, obsd. 567.4 [M+H, ES⁺].

### Step 2: {4′-[4-Ethyl-5-((R)-1-(3-trifluoromethyl-phenyl-ethoxycarbonylamino)[1,2,3]triazol-1-yl]-biphenyl-4-yl}-acetic acid

To a solution of {4′-[4-ethyl-5-((R)-1-(3-trifluoromethyl-phenyl-ethoxycarbonylamino)[1,2,3]triazol-1-yl]-biphenyl-4-yl}-acetic acid ethyl ester (185 mg, 0.33 mmol) in THF (5 mL) and ethanol (5 mL) was added an excess of 1.0 N sodium hydroxide (3.27 mL, 3.27 mmol) solution in water at room temperature. The resulting colorless solution was stirred for 15 h at room temperature at which time TLC analysis indicated the absence of starting material. Then, it was diluted with water (˜15 mL) and the solvent was removed under vacuum. The basic aqueous layer was neutralized with 1.0 N HCl. The resulting precipitate was extracted into EA (2×45 mL) and the combined extracts were washed with brine solution. After drying over anhydrous MgSO₄ and filtration, the solvent was removed under vacuum to obtain the desired acid which was dissolved in dichloromethane (˜5 mL) and then diluted with hexanes. As a result, solids were formed and they were collected by filtration and washed with hexanes. After air drying, 135 mg (77% yield) of {4′-[4-ethyl-5-((R)-1-(3-trifluoromethyl-phenyl-ethoxycarbonylamino)-[1,2,3]triazol-1-yl]-biphenyl-4-yl}-acetic acid was isolated as a white solid. LC/MS calcd. for C₂₈H₂₅F₃N₄O₄ (m/e) 538, obsd. 539.3 [M+H, ES⁺]. ¹H NMR (DMSO-d₆) δ: 12.40 (s, 1H), 9.30-9.92 (m, 1H), 7.83 (d, J=6.8 Hz, 2H), 7.53-7.76 (m, 7H), 7.41 (d, J=8.0 Hz, 3H), 5.79 (d, J=15.6 Hz, 1H), 3.66 (s, 2H), 2.55 (d, J=7.3 Hz, 2H), 1.51 (br. s., 3H), 1.18 (t, J=7.4 Hz, 3H).

### Example 21

### 1-{4′-[5-((R)-1-Phenyl-ethoxycarbonylamino)-[1,2,3]triazol-1-yl]-biphenyl-4-yl}-cyclopropanecarboxylic acid

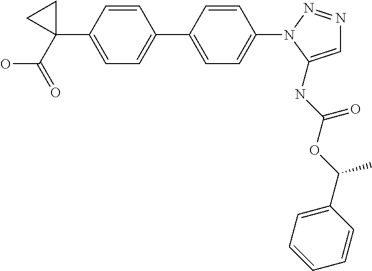 [SMILES: C[C@@H](OC(=O)Nc1cnnn1-c1ccc(-c2ccc(C3(C(=O)O)CC3)cc2)cc1)c1ccccc1]

### Step 1: 3-(4-Bromo-phenyl)-3H-[1,2,3]triazole-4-carboxylic acid methyl ester

In 350 mL reaction vial 1-azido-4-bromo-benzene (5 g, 25.2 mmol) and propionic acid methyl ester (2.12 g, 2.11 mL, 25.2 mmol) were combined with Toluene (50 mL) to give a yellow suspension. The vial was sealed and heated in an oil bath at 150° C. for 5.5 h. The reaction was filtered, solid washed with toluene and EtOAc. The filtrate was concentrated, dissolved in minimal DCM, and purified by flash chromatography (silica gel, 0% to 50% EtOAc in hexanes). Appropriate fractions combined, concentrated, and dried from DCM/hexanes yielding 3-(4-bromo-phenyl)-3H-[1,2,3]triazole-4-carboxylic acid methyl ester (1.5 g, 21.1% yield) as a light brown solid. LC/MS calcd. for C₁₀H₈BrN₃O₂ (m/e) 281/283, obsd. 282/284 (M+H, ES⁺).

### Step 2: 3-(4-Bromo-phenyl)-3H-[1,2,3]triazole-4-carboxylic acid

To 200 mL round bottom flask containing 3-(4-bromo-phenyl)-3H-[1,2,3]triazole-4-carboxylic acid methyl ester (1.0 g, 3.54 mmol) dissolved in THF (30 mL) (brown solution) was added LiOH (0.81 g, 34 mmol) in water (10 mL) with heat to partially dissolve. The solution was stirred at room temperature for 20 h. The reaction was concentrated, diluted in water (total volume, 200 mL) extracted with ethyl ether (2×100 mL). The aqueous layer was acidified with 1 N HCl and the resulting precipitate was filtered, washed with water and hexanes, and dried over house vacuum yielding 3-(4-bromo-phenyl)-3H-[1,2,3]triazole-4-carboxylic acid (0.78 g, 81.7% yield) as a light brown solid. LC/MS calcd. for C₉H₆N₃O₂ (m/e) 267/269, obsd. 268/270 (M+H, ES⁺).

### Step 3: [3-(4-Bromo-phenyl)-3H-[1,2,3]triazol-4-yl]-carbamic acid (R)-1-phenyl-ethyl ester

In a 40 mL reaction vial, 3-(4-bromo-phenyl)-3H-[1,2,3]triazole-4-carboxylic acid (399 mg, 1.45 mmol), (R)-1-phenylethanol (265 mg, 0.83 mL, 2.17 mmol) and triethylamine (293 mg, 0.4 mL, 2.9 mmol) were combined with toluene (17 mL) to give a yellow solution and to this was added diphenylphosphorylazide (797 mg, 0.624 mL, 2.89 mmol). The vial's atmosphere was purged with nitrogen, sealed, heated in an oil bath at 65° C. for 3.5 h, and cooled to room temperature overnight. The reaction was concentrated, diluted with EtOAc, washed with Water and brine, and dried over MgSO₄, filtered, concentrated, dissolved in minimal DCM, and purified by flash chromatography (silica gel, 0% to 50% EtOAc in hexanes). Appropriate fractions combined, concentrated, and dried from DCM/hexanes, yielding [3-(4-bromo-phenyl)-3H-[1,2,3]triazol-4-yl]-carbamic acid (R)-1-phenyl-ethyl ester (408 mg, 72.8% yield) as a white solid. LC/MS calcd. for C₁₇H₁₅BrN₄O₂ (m/e) 386/388, obsd. 387/389 (M+H, ES⁺).

### Step 4: 1-{4′-[5-((R)-1-Phenyl-ethoxycarbonylamino)-[1,2,3]triazol-4-yl]-biphenyl-4-yl}-cyclopropanecarboxylic acid methyl ester

In a 20 mL vial, 1-[4-(4,4,5,5-tetramethyl-[1,3,2]dioxaborolan-2-yl)-phenyl]-cyclopropanecarboxylic acid methyl ester (93.6 mg, 0.310 mmol), [3-(4-bromo-phenyl)-3H[1,2,3]triazol-4-yl]-carbamic acid (R)-1-phenyl-ethyl ester (100 mg, 0.258 mmol), 2-dicyclohexyphosphino-2′,6′-dimethoxybiphenyl (SPhos) (31.8 mg, 0.0775 mmol), Pd(OAc)₂ (8.7 mg, 0.039 mmol), and tripotassium phosphate (164 mg, 0.0775 mmol) were combined with toluene (8 mL) and water (2 mL) (previously purged with nitrogen for 20 min) to give a light yellow solution. The vial's atmosphere was replaced with nitrogen, sealed, heated in a dry block at 80° C. 4 h, and cooled to room temperature overnight. The reaction was diluted with EtOAc (70 mL) and washed with water (100 mL) and brine (50 ml). The aqueous layers were extracted with EtOAc (60 mL). The organic layers were combined, dried over MgSO₄, filtered, concentrated, dissolved in minimal DCM, and purified by flash chromatography (silica gel, 0% to 50% EtOAc in hexanes). Appropriate fractions combined, concentrated, and dried from DCM/hexanes yielding 1-{4′-[5-((R)-1-phenyl-ethoxycarbonylamino)-[1,2,3]triazol-1-yl]-biphenyl-4-yl}-cyclopropanecarboxylic acid methyl ester (45.1 mg, 36.2% yield) as a light yellow solid. LC/MS calcd. for C₂₈H₂₆N₄O₄ (m/e) 482, obsd. 483 (M+H, ES⁺).

### Step 5: 1-{4′-[5-((R)-1-Phenyl-ethoxycarbonylamino)-[1,2,3]triazol-1-yl]-biphenyl-4-yl}-cyclopropanecarboxylic acid

In a 200 mL round-bottomed flask, 1-{4′-[5-((R)-1-phenyl-ethoxycarbonylamino)[1,2,3]triazol-1-yl]-biphenyl-4-yl}-cyclopropanecarboxylic acid methyl ester (40 mg, 0.0829 mmol) was combined with THF (2 mL) and MeOH (2 mL) to give a yellow solution. To this was dripped in NaOH (1 M, 1 mL, 1 mmol). The reaction was stirred at room temperature overnight. The reaction was diluted with water, concentrated, diluted with more water and acidified with 1 N HCl. The resulting precipitate was filtered, washed with water and hexanes, and dried over house vacuum and in a desiccator yielding 1-{4′-[5-((R)-1-phenyl-ethoxycarbonylamino)-[1,2,3]triazol-1-yl]-biphenyl-4-yl}-cyclopropanecarboxylic acid (28.7 mg, 73.9% yield) as a light brown solid. LC/MS calcd. for C₂₇H₂₄N₄O₄ (m/e) 468, obsd. 469 (M+H, ES⁺). ¹H NMR (DMSO-d₆) δ: 12.38 (br. s., 1H), 10.04 (br. s., 1H), 7.87 (d, J=8.3 Hz, 2H), 7.82 (s, 1H), 7.68 (d, J=8.3 Hz, 2H), 7.62 (d, J=8.3 Hz, 2H), 7.48 (d, J=8.0 Hz, 2H), 7.17-7.41 (m, 5H), 5.74 (d, J=5.8 Hz, 1H), 1.34-1.62 (m, 5H), 1.21 (d, J=2.5 Hz, 2H).

### Example 22

### {4′-[5-((R)-1-Phenyl-ethoxycarbonylamino)-[1,2,3]triazol-1-yl]-biphenyl-4-yl}-acetic acid

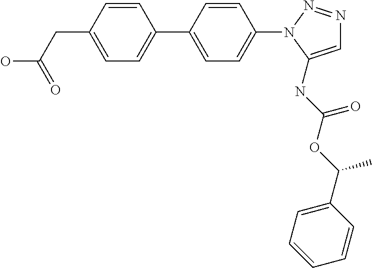 [SMILES: C[C@@H](OC(=O)Nc1cnnn1-c1ccc(-c2ccc(CC(=O)O)cc2)cc1)c1ccccc1]

### Step 1: {4′-[5-((R)-1-Phenyl-ethoxycarbonylamino)-[1,2,3]triazol-1-yl]-biphenyl-4-yl}-acetic acid ethyl ester

In a 20 mL vial, 1-[4-(4,4,5,5-tetramethyl-[1,3,2]dioxaborolan-2-yl)-phenyl]-acetic acid ethyl ester (89.9 mg, 0.310 mmol), [3-(4-bromo-phenyl)-3H-[1,2,3]triazol-4-yl]-carbamic acid (R)-1-phenyl-ethyl ester (100 mg, 0.258 mmol), 2-dicyclohexyphosphino-2′,6′-dimethoxybiphenyl (SPhos) (31.8 mg, 0.0775 mmol), Pd(OAc)₂ (8.7 mg, 0.039 mmol), and tripotassium phosphate (164 mg, 0.0775 mmol) were combined with toluene (7 mL) and water (2 mL) (previously purged with nitrogen for 20 min) to give a light yellow solution. The vial's atmosphere was purged with nitrogen, sealed, heated in a dry block at 80° C. for 4 h, and cooled to room temperature overnight. Additional 1-[4-(4,4,5,5-tetramethyl-[1,3,2]dioxaborolan-2-yl)-phenyl]-acetic acid ethyl ester (89.9 mg, 0.310 mmol), 2-dicyclohexyphosphino-2′,6′-dimethoxybiphenyl (SPhos) (31.8 mg, 0.0775 mmol), and Pd(OAc)₂ (8.7 mg, 0.039 mmol) were added. The vial's atmosphere was purged with nitrogen, sealed, heated in a dry block at 80° C. for 4 h, and cooled to room temperature overnight. The reaction was diluted with EtOAc (70 mL) and washed with water (100 mL) and brine (50 ml). The aqueous layers were extracted with EtOAc (60 mL) and the organic layers were combined, dried over MgSO₄, filtered, concentrated, dissolved in minimal DCM and purified by flash chromatography (silica gel, 0% to 50% EtOAc in hexanes). Appropriate fractions combined, concentrated, and dried from DCM/hexanes yielding {4′-[5-((R)-1-phenyl-ethoxycarbonylamino)-[1,2,3]triazol-1-yl]-biphenyl-4-yl}-acetic acid ethyl ester (46 mg, 37.9% yield) as a light yellow solid. LC/MS (ES) calcd. for C₂₇H₂₆N₄O₄ (m/e) 470, obsd. 471 (M+H, ES⁺).

### Step 2: {4′-[5-((R)-1-Phenyl-ethoxycarbonylamino)-[1,2,3]triazol-1-yl]-biphenyl-4-yl}-acetic acid

In a 200 mL round-bottomed flask, 1-{4′-[5-((R)-1-phenyl-ethoxycarbonylamino)[1,2,3]triazol-1-yl]-biphenyl-4-yl}-cyclopropanecarboxylic acid methyl ester (41 mg, 0.087 mmol) was combined with THF (2 mL) and MeOH (2 mL) to give a yellow solution. To this was dripped in NaOH (1 M, 1 mL, 1 mmol). The reaction was stirred at room temperature overnight. The reaction was diluted with water, concentrated, diluted with more water, and acidified with 1 N HCl. The resulting precipitate was filtered, washed with water and hexanes, and dried over house vacuum and in a desiccator yielding {4′-[5-((R)-1-phenyl-ethoxycarbonylamino)-[1,2,3]triazol-1-yl]-biphenyl-4-yl}-acetic acid (28.6 mg, 74.2% yield) as a yellow solid. LC/MS calcd. for C₂₅H₂₂N₄O₄ (m/e) 442, obsd. 443 (M+H, ES⁺). ¹H NMR (DMSO-d₆) δ: 12.39 (br. s., 1H), 10.03 (br. s., 1H), 7.88 (d, J=8.3 Hz, 2H), 7.82 (s, 1H), 7.72 (d, J=8.0 Hz, 2H), 7.62 (d, J=8.3 Hz, 2H), 7.42 (d, J=8.0 Hz, 2H), 7.13-7.39 (m, 5H), 5.74 (d, J=5.3 Hz, 1H), 3.66 (s, 2H), 1.46 (br. s., 3H).

### Example 23

### 2-Methyl-2-{4′-[4-methyl-5-((R)-1-phenyl-ethoxycarbonylamino)-[1,2,3]triazol-1-yl]-biphenyl-4-yl}-propionic acid

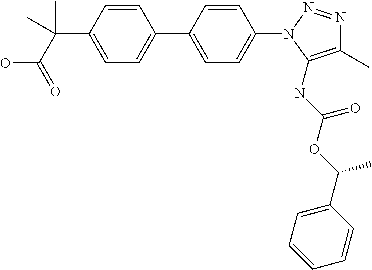 [SMILES: Cc1nnn(-c2ccc(-c3ccc(C(C)(C)C(=O)O)cc3)cc2)c1NC(=O)O[C@H](C)c1ccccc1]

### Step 1: {5-Methyl-3-[4-(4,4,5,5-tetramethyl-[1,3,2]dioxaborolan-2-yl)-phenyl]-3H[1,2,3]triazol-4-yl}-carbamic acid (R)-1-phenyl-ethyl ester

In a 20 mL vial, (R)-1-phenylethyl 1-(4-bromophenyl)-4-methyl-1H-1,2,3-triazol-5-ylcarbamate (2.39 g, 5.96 mmol), BISPIN (1.82 g, 7.15 mmol) and potassium acetate (1.17 g, 11.9 mmol) were combined with 1,4-dioxane (59.8 mL) to give a white suspension that was purged with nitrogen for 5 min. To the mixture was added PdCl₂(DPPF) (0.486 g, 0.596 mmol. The vial was sealed, stirred in a dry block at 80° C. for 3.5 h, and cooled to room temperature overnight. The reaction was filtered, rinsed with EtOAc, concentrated, diluted with EtOAc (200 mL), and filtered again. The filtrate was washed with water (200 mL) and brine (100 mL). The aqueous layers were extracted with EtOAc (200 mL). The organic layers were combined, dried over MgSO₄, filtered, concentrated, and the crude material was purified by flash chromatography (silica gel, 0% to 50% EtOAc in hexanes). Appropriate fractions combined, yielding {5-methyl-3-[4-(4,4,5,5-tetramethyl-[1,3,2]dioxaborolan-2-yl)phenyl]-3H-[1,2,3]triazol-4-yl}-carbamic acid (R)-1-phenyl-ethyl ester (2.24 g, 83% yield) as a clear oil that solidifies as a white crystal upon cooling to room temperature. LC/MS calcd. for C₂₄H₂₉BN₄O₄ (m/e) 448, obsd. 449 (M+H, ES⁺).

### Step 2: 2-Methyl-2-{4′-[4-methyl-5-((R)-1-phenyl-ethoxycarbonylamino)-[1,2,3]triazol-1-yl]-biphenyl-4-yl}-propionic acid methyl ester

In a 20 mL vial, 2-(4-bromo-phenyl)-2-methyl-propionic acid methyl ester (130 mg, 0.506 mmol), {5-methyl-3-[4-(4,4,5,5-tetramethyl-[1,3,2]dioxaborolan-2-yl)-phenyl]-3H[1,2,3]triazol-4-yl}-carbamic acid (R)-1-phenyl-ethyl ester (212 mg, 0.473 mmol), 2-dicyclohexyphosphino-2′,6′-dimethoxybiphenyl (SPhos) (59 mg, 0.144 mmol), tripotassium phosphate (292 mg, 1.38 mmol), and Pd(OAc)₂ (17 mg, 0.0.75 mmol) were combined with toluene (8 mL) and water (2 mL) (previously purged with nitrogen for 20 min) to give a light yellow suspension. The vial's atmosphere was purged with nitrogen, sealed, heated in dry block at 80° C. for 4 h, and cooled to room temperature overnight. The reaction was diluted with EtOAc, washed with water and brine, dried over MgSO₄, filtered, concentrated, dissolved in minimal DCM and purified by flash chromatography (silica gel, 0% to 50% EtOAc in hexanes). Appropriate fractions combined, concentrated, and dried from DCM/hexanes yielding 2-methyl-2-{4′-[4-methyl-5-((R)-1-phenyl-ethoxycarbonylamino)[1,2,3]triazol-1-yl]-biphenyl-4-yl}-propionic acid methyl ester (106 mg, 45% yield) as a white solid. LC/MS calcd. for C₂₉H₃₀N₄O₄ (m/e) 498, obsd. 499 (M+H, ES⁺).

### Step 3: 2-Methyl-2-{4′-[4-methyl-5-((R)-1-phenyl-ethoxycarbonylamino)-[1,2,3]triazol-1-yl]-biphenyl-4-yl}-propionic acid

In a 200 mL round-bottomed flask, 2-methyl-2-{4′-[4-methyl-5-((R)-1-phenyl-ethoxycarbonylamino)-[1,2,3]triazol-1-yl]-biphenyl-4-yl}-propionic acid methyl ester (100 mg, 0.201 mmol) was combined with THF (4 mL) and MeOH (4 mL) to give a yellow solution. To this was dripped in 1 M NaOH (2 mL, 2.0 mmol). The reaction was stirred at room temperature for 2 days and stored in a refrigerator for 1.5 days. The reaction was stirred again at room temperature for 1 day and then more 1 M NaOH (1 ml, 1 mmol) was added. The reaction was heated in a dry block at 40° C. for 6 h and then cooled to room temperature overnight. The reaction was diluted with water, concentrated, diluted with more water and acidified with 1 N HCl. The resulting precipitate was filtered, washed with water and hexanes, and dried over house vacuum and in a desiccator yielding 2-methyl-2-{4′-[4-methyl-5-((R)-1-phenyl-ethoxycarbonylamino)-[1,2,3]triazol-1-yl]-biphenyl-4-yl}-propionic acid (79.6 mg, 81.9% yield) as a white solid. LC/MS calcd. for C₂₈H₂₈N₄O₄ (m/e) 484, obsd. 485 (M+H, ES⁺). ¹H NMR (DMSO-d₆) δ: 12.43 (br. s., 1H), 9.68 (br. s., 1H), 7.84 (d, J=7.0 Hz, 2H), 7.71 (d, J=8.3 Hz, 2H), 7.58 (d, J=7.5 Hz, 2H), 7.50 (d, J=8.3 Hz, 2H), 7.06-7.43 (m, 5H), 5.70 (br. s., 1H), 2.18 (s, 3H), 1.33-1.73 (m, 9H).

### Example 24

### (R)-1-(4′-(4-Methyl-5-((1-phenylethoxy)carbonylamino)-1H-1,2,3-triazol-1-yl)biphenyl-3-yl)cyclopropanecarboxylic acid

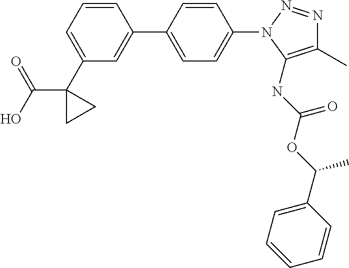 [SMILES: Cc1nnn(-c2ccc(-c3cccc(C4(C(=O)O)CC4)c3)cc2)c1NC(=O)O[C@H](C)c1ccccc1]

### Step 1: 1-(3-(4,4,5,5-Tetramethyl-1,3,2-dioxaborolan-2-yl)phenyl)cyclopropane carboxylic acid ethyl ester

A 350 mL sealed cap vessel was charged with 1-(3-bromophenyl)cyclopropanecarboxylic acid ethyl ester (3.56 g, 13.2 mmol), 4,4,4′,4′,5,5,5′,5′-octamethyl-2,2′-bi(1,3,2-dioxaborolane) (4.03 g, 15.9 mmol), and potassium acetate (2.6 g, 26.5 mmol) and then 1,4-Dioxane (40 mL) was added to give a white suspension. The mixture was then nitrogen gas was bubbled through the reaction mixture for 10 minutes before the addition of [1,1′-bis(diphenylphosphino)ferrocene]dichloropalladium(II) (484 mg, 0.66 mmol) at room temperature under nitrogen atmosphere. Then, the flask was sealed with a cap and the brown reaction mixture was heated in an oil bath at 80° C. for 5 h. Then, it was cooled to room temperature and poured into a solution of water (100 mL) and brine (100 mL) and the organic compound was extracted into EA (2×150 mL) (it was difficult to see the two layers because of the black mixture). The combined extracts were washed with brine solution and dried over anhydrous MgSO₄. Filtration and concentration gave the crude black oil (˜11.11 g) which was purified using an ISCO (120 g) column chromatography eluting with 0-60% EA in hexanes. The desired fractions (20-40) were combined and the solvent was removed under vacuum to obtain 1-(3-(4,4,5,5-tetramethyl-1,3,2-dioxaborolan-2-yl)phenyl)cyclopropane carboxylic acid ethyl ester as viscous oil (2.55 g, 61% yield). LC/MS calcd. for C₁₈H₂₅BO₄ (m/e) 316, obsd. 317.2 [M+H, ES⁺].

### Step 2: (R)-1-(4′-(4-Methyl-5-((1-phenylethoxy)carbonylamino)-1H-1,2,3-triazol-1-yl)biphenyl-3-yl)cyclopropanecarboxylic acid ethyl ester

To a mixture of ethyl 1-(3-(4,4,5,5-tetramethyl-1,3,2-dioxaborolan-2-yl)phenyl)cyclopropanecarboxylate (236 mg, 0.75 mmol), (R)-1-phenylethyl 1-(4-bromophenyl)-4-methyl-1H-1,2,3-triazol-5-ylcarbamate (200 mg, 0.5 mmol), palladium(II) acetate (16.8 mg, 0.075 mmol), 2-dicyclohexylphosphino-2′,6′-dimethoxybiphenyl (61.4 mg, 0.15 mmol), and tripotassium phosphate (317 mg, 1.5 mmol) in a 50 mL sealed tube were added freshly degassed toluene (4.5 mL) and water (1.0 mL) at room temperature under nitrogen atmosphere. Then, the rubber septum was replaced with a cap and the resulting light yellow suspension was heated to 110° C. with oil bath. During this period, it turned to a black suspension. Then, the reaction mixture was cooled to room temperature and poured into water and brine solution. The organic compound was extracted into EA (2×50 mL) and the combined extracts were washed with brine solution and dried over anhydrous MgSO₄. Filtration and concentration gave the crude product which was purified using an ISCO (80 g) column chromatography eluting with 0-100% EA in hexanes. The desired fractions were combined and the solvent was removed under vacuum to obtain (R)-1-(4′-(4-methyl-5-((1-phenylethoxy)carbonylamino)-1H-1,2,3-triazol-1-yl)biphenyl-3-yl)cyclopropanecarboxylic acid ethyl ester as an amorphous solid (144 mg, 56.6% yield). LC/MS calcd. for C₃₀H₃₀N₄O₄ (m/e) 510, obsd. 511.2 [M+H, ES⁺].

### Step 3: (R)-1-(4′-(4-Methyl-5-((1-phenylethoxy)carbonylamino)-1H-1,2,3-triazol-1-yl)biphenyl-3-yl)cyclopropanecarboxylic acid

To a solution of (R)-1-(4′-(4-methyl-5-((1-phenylethoxy)carbonylamino)-1H-1,2,3-triazol-1-yl)biphenyl-3-yl)cyclopropanecarboxylic acid ethyl ester (162 mg, 0.32 mmol) in ethanol (6 mL) was added an excess of 1 N sodium hydroxide (1.59 mL, 1.59 mmol) solution in water at room temperature. Then, the resulting cloudy solution was stirred for 20 h at which time LCMS analysis indicated the presence of still some starting material. Then, the cloudy reaction mixture was heated in an oil bath to 55° C. and stirred for 3 h at which time LCMS analysis indicated the absence of starting material. Then, it was cooled to room temperature and the solvent was removed under vacuum and the residue was diluted with water. The basic aqueous layer was neutralized with 1 N HCl. The resulting solids were collected by filtration and washed with water. After air drying, 130 mg (81.6% yield) of (R)-1-(4′-(4-methyl-5-((1-phenylethoxy)carbonylamino)-1H-1,2,3-triazol-1-yl)biphenyl-3-yl)cyclopropanecarboxylic acid was isolated as a white solid. LC/MS calcd. for C₂₈H₂₆N₄O₄ (m/e) 482, obsd. 483.1 [M+H, ES⁺]. ¹H NMR (DMSO-d₆) δ: 12.39 (br. s., 1H), 9.32-10.31 (m, 1H), 7.80 (d, J=6.8 Hz, 2H), 7.70 (br. s., 2H), 7.53-7.64 (m, 2H), 7.39-7.52 (m, 4H), 7.35 (d, J=7.5 Hz, 3H), 5.80 (br. s., 1H), 3.05-3.57 (m, 3H), 1.35-1.74 (m, 5H), 1.25 (br. s., 2H).

### Example 25

### 1-{3′-[4-Methyl-5-((R)-1-phenyl-ethoxycarbonylamino)-[1,2,3]triazol-1-yl]-biphenyl-4-yl}-cyclopropanecarboxylic acid

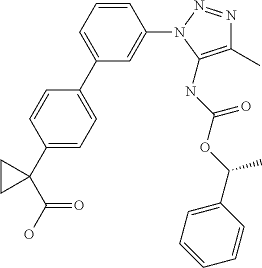 [SMILES: Cc1nnn(-c2cccc(-c3ccc(C4(C(=O)O)CC4)cc3)c2)c1NC(=O)O[C@H](C)c1ccccc1]

### Step 1: 3-(3-Bromo-phenyl)-5-methyl-3H-[1,2,3]triazole-4-carboxylic acid methyl ester

In 350 mL reaction vial 1-azido-3-bromo-benzene (2.47 g, 12.5 mmol) and methyl but-2-ynoate (1.35 g, 1.37 mL, 13.7 mmol) were combined with Toluene (106 mL) to give a yellow suspension. The vial was sealed and heated in an oil bath at 150° C. accidentally for 2.5 day (4 h intended). The reaction was filtered, solid washed with toluene. The filtrate was concentrated, dissolved in minimal DCM, and purified by flash chromatography (silica gel, 0% to 30% EtOAc in hexanes). Appropriate fractions combined, concentrated, and dried from DCM/hexanes yielding 3-(3-bromo-phenyl)-5-methyl-3H-[1,2,3]triazole-4-carboxylic acid methyl ester (1.04 g, 28.2% yield) as a light brown solid. LC/MS calcd. for C₁₁H₁₀BrN₃O₂ 295/297, obsd. 296/298 (M+H, ES⁺).

### Step 2: 3-(3-Bromo-phenyl)-5-methyl-3H-[1,2,3]triazole-4-carboxylic acid

To 250 mL round bottom flask containing 3-(3-bromo-phenyl)-5-methyl-3H-[1,2,3]triazole-4-carboxylic acid methyl ester (1.0 g, 3.38 mmol) dissolved in THF (40 mL) (brown solution) was added LiOH (0.81 g, 34 mmol) in water (10 mL) with heat to partially dissolve.

The solution was stirred at room temperature overnight. The reaction was concentrated, diluted in water (total volume, 200 mL) extracted with ethyl ether (2×100 mL). The aqueous layer was acidified with 1 N HCl. The resulting precipitate was filtered, washed with water and hexanes, and dried over house vacuum and in a desiccator yielding 3-(3-bromo-phenyl)-5-methyl-3H-[1,2,3]triazole-4-carboxylic acid (0.913 g, 95.8% yield) as a light brown solid. LC/MS calcd. for C₁₀H₈N₃O₂ (m/e) 281/283, obsd. 281/284 (M+H, ES⁺).

### Step 3: [3-(3-Bromo-phenyl)-5-methyl-3H-[1,2,3]triazol-4-yl]-carbamic acid (R)-1-phenyl-ethyl ester

In a 40 mL reaction vial, 3-(3-bromo-phenyl)-5-methyl-3H-[1,2,3]triazole-4-carboxylic acid (0.91 g, 3.2 mmol), (R)-1-phenylethanol (0.84 g, 0.83 mL, 6.9 mmol) and triethylamine (0.91 g, 1.3 mL, 9.0 mmol) were combined with toluene (28 mL) to give a yellow solution and to this was added diphenylphosphorylazide (2.5 g, 1.9 mL, 9.0 mmol). The vial's atmosphere was purged with nitrogen, sealed, heated in an oil bath at 65° C. for 2.5 h, and cooled to room temperature overnight. The reaction was concentrated as yellow viscous oil, diluted with DCM, and purified by flash chromatography (silica gel, 0% to 50% EtOAc in hexanes). Appropriate fractions combined, concentrated, dried from DCM/hexanes, yielding [3-(3-bromo-phenyl)-5-methyl-3H-[1,2,3]triazol-4-yl]-carbamic acid (R)-1-phenyl-ethyl ester (0.86 g, 66% yield) as a light yellow solid/gum. LC/MS calcd. for C₁₈H₁₇BrN₄O₂ (m/e) 400/402, obsd. 401/403 (M+H, ES⁺).

### Step 4: 1-{3′-[4-Methyl-5-((R)-1-phenyl-ethoxycarbonylamino)-[1,2,3]triazol-1-yl]-biphenyl-4-yl}-cyclopropanecarboxylic acid methyl ester

In a 40 mL vial, 1-[4-(4,4,5,5-tetramethyl-[1,3,2]dioxaborolan-2-yl)-phenyl]-cyclopropanecarboxylic acid methyl ester (356 mg, 1.18 mmol), [3-(3-bromo-phenyl)-5-methyl-3H-[1,2,3]triazol-4-yl]-carbamic acid (R)-1-phenyl-ethyl ester (430 mg, 1.07 mmol), 2-dicyclohexyphosphino-2′,6′-dimethoxybiphenyl (SPhos) (132 mg, 0.321 mmol), and Pd(OAc)₂ (36.1 mg, 0.161 mmol) were combined with toluene (34 mL) (previously purged with nitrogen for 20 min) to give a light yellow solution. To this was added tripotassium phosphate (682 mg, 3.21 mmol) dissolved in water (9 mL) (previously purged with nitrogen for 20 min). The vial's atmosphere was purged with nitrogen, sealed, heated in oil bath at 100° C. accidentally for 2.5 days (intended 4 h) and cooled to room temperature in 1 h. The reaction was filtered, diluted with EtOAc (50 mL) and washed with water/brine (100/50 mL) and brine (150 ml). The aqueous layers were extracted with EtOAc (2×150 mL). The organic layers were combined, dried over MgSO₄, filtered, concentrated, dissolved in minimal DCM, and purified by flash chromatography (silica gel, 0% to 100% EtOAc in hexanes). Appropriate fractions combined, concentrated, and dried from DCM/hexanes yielding 1-{3′-[4-methyl-5-((R)-1-phenyl-ethoxycarbonylamino)-[1,2,3]triazol-1-yl]-biphenyl-4-yl}-cyclopropanecarboxylic acid methyl ester (142 g, 26.7% yield) as a white solid. LC/MS calcd. for C₂₉H₂₈N₄O₄ (m/e) 496, obsd. 497 (M+H, ES⁺).

### Step 5: 1-{3′-[4-Methyl-5-((R)-1-phenyl-ethoxycarbonylamino)-[1,2,3]triazol-1-yl]-biphenyl-4-yl}-cyclopropanecarboxylic acid

In a 100 mL round-bottomed flask, 1-{3′-[4-methyl-5-((R)-1-phenyl-ethoxycarbonylamino)[1,2,3]triazol-1-yl]-biphenyl-4-yl}-cyclopropanecarboxylic acid methyl ester (141 mg, 0.284 mmol) was combined with THF (10 mL) to give a colorless solution. To this was dripped in NaOH (1 M, 2.8 mL, 2.8 mmol). The reaction was stirred at room temperature and additional water and THF were added. After 18 h, the reaction was diluted with water, concentrated, diluted with more water and acidified with 1 N HCl. The resulting precipitate was filtered, washed with water and hexanes, and dried over house vacuum yielding 1-{3′-[4-methyl-5-((R)-1-phenyl-ethoxycarbonylamino)-[1,2,3]triazol-1-yl]-biphenyl-4-yl}-cyclopropanecarboxylic acid (49 mg, 35.8% yield) as a white solid. LC/MS calcd. for C₂₈H₂₆N₄O₄ (m/e) 482, obsd. 483 (M+H, ES⁺). ¹H NMR (DMSO-d₆) δ: 12.39 (br. s., 1H), 9.72 (br. s., 1H), 7.84 (d, J=7.8 Hz, 1H), 7.77 (s, 1H), 7.56-7.73 (m, 3H), 7.51 (d, J=7.3 Hz, 1H), 7.43 (d, J=8.0 Hz, 2H), 7.28 (br. s., 5H), 5.70 (br. s., 1H), 2.12-2.27 (m, 3H), 1.27-1.65 (m, 5H), 1.19 (d, J=2.8 Hz, 2H).

### Example 26

### {3′-[4-Methyl-5-((R)-1-phenyl-ethoxycarbonylamino)-[1,2,3]triazol-1-yl]-biphenyl-4-yl}-acetic acid

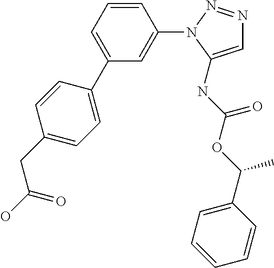 [SMILES: C[C@@H](OC(=O)Nc1cnnn1-c1cccc(-c2ccc(CC(=O)O)cc2)c1)c1ccccc1]

### Step 1: {3′-[4-Methyl-5-((R)-1-phenyl-ethoxycarbonylamino)-[1,2,3]triazol-1-yl]-biphenyl-4-yl}-acetic acid ethyl ester

In a 40 mL vial, 1-[4-(4,4,5,5-tetramethyl-[1,3,2]dioxaborolan-2-yl)-phenyl]-acetic acid ethyl ester (311 mg, 1.07 mmol), [3-(3-bromo-phenyl)-5-methyl-3H-[1,2,3]triazol-4-yl]-carbamic acid (R)-1-phenyl-ethyl ester (430 mg, 1.07 mmol), 2-dicyclohexyphosphino-2′,6′-dimethoxybiphenyl (SPhos) (132 mg, 0.321 mmol), and Pd(OAc)₂ (36.1 mg, 0.161 mmol) were combined with toluene (12 mL) (previously purged with nitrogen for 20 min) to give a light yellow solution. To this was added tripotassium phosphate (682 mg, 3.21 mmol) dissolved in water (4 mL) (previously purged with nitrogen for 20 min). The vial's atmosphere was replaced with nitrogen, sealed, heated in oil bath at 100° C. accidentally for 2.5 days (intended 4 h) and cooled to room temperature in 1 h. The reaction was filtered, diluted with EtOAc (50 mL) and washed with water/brine (100/50 mL) and brine (150 ml). The aqueous layers were extracted with EtOAc (2×150 mL). The organic layers were combined, dried over MgSO₄, filtered, concentrated, dissolved in minimal DCM, and purified by flash chromatography (silica gel, 0% to 100% EtOAc in hexanes). Appropriate fractions combined, concentrated, and dried from DCM/hexanes yielding {3′-[4-methyl-5-((R)-1-phenyl-ethoxycarbonylamino)-[1,2,3]triazol-1-yl]-biphenyl-4-yl}-acetic acid ethyl ester (96.7 mg, 18.6% yield) as a white solid. LC/MS calcd. for C₂₈H₂₈N₄O₄ (m/e) 484, obsd. 485 (M+H, ES⁺).

### Step 2: {3′-[4-Methyl-5-((R)-1-phenyl-ethoxycarbonylamino)-[1,2,3]triazol-1-yl]-biphenyl-4-yl}-acetic acid

In a 100 mL round-bottomed flask, {3′-[4-methyl-5-((R)-1-phenyl-ethoxycarbonylamino)[1,2,3]triazol-1-yl]-biphenyl-4-yl}-acetic acid ethyl ester (90 mg, 0.186 mmol) was combined with THF (5 mL) to give a colorless solution. To this was dripped in LiOH (78 mg, 1.86 mmol) in water (1 mL) with heat to partially dissolve. The reaction was stirred at room temperature for 17 h. The reaction was diluted with water, concentrated, diluted with more water and acidified with 1 N HCl. The resulting precipitate was filtered, washed with water and hexanes, and dried over house vacuum yielding 1-{3′-[4-methyl-5-((R)-1-phenyl-ethoxycarbonylamino)-[1,2,3]triazol-1-yl]-biphenyl-4-yl}-cyclopropanecarboxylic acid (64.7 mg, 76.3% yield) as a white solid. LC/MS calcd. for C₂₆H₂₄N₄O₄ (m/e) 456, obsd. 457 (M+H, ES⁺). ¹H NMR (DMSO-d₆) δ: 12.41 (br. s., 1H), 9.72 (br. s., 1H), 7.85 (d, J=7.8 Hz, 1H), 7.78 (s, 1H), 7.59-7.73 (m, 3H), 7.51 (d, J=7.5 Hz, 1H), 7.39 (d, J=8.0 Hz, 2H), 7.06-7.34 (m, 5H), 5.69 (br. s., 1H), 3.65 (s, 2H), 2.19 (s, 3H), 1.44 (br. s., 3H).

### Example 27

### (3-Biphenyl-4-yl-5-methyl-3H-[1,2,3]triazol-4-yl)-carbamic acid (R)-1-phenyl-ethyl ester

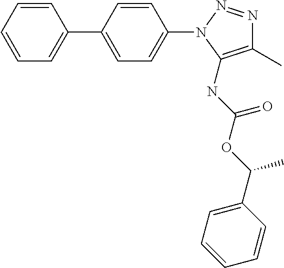 [SMILES: Cc1nnn(-c2ccc(-c3ccccc3)cc2)c1NC(=O)O[C@H](C)c1ccccc1]

In a 20 mL vial, phenylboronic acid (6.9 mg, 0.057 mmol), [3-(4-bromo-phenyl)-5-methyl-3H-[1,2,3]triazol-4-yl]-carbamic acid tert-butyl ester (18.9 mg, 0.0471 mmol), 2-dicyclohexyphosphino-2′,6′-dimethoxybiphenyl (SPhos) (7.7 mg, 0.019 mmol), tripotassium phosphate (30 mg, 0.14 mmol), and Pd(OAc)₂ (2.0 mg, 0.0089 mmol) were combined with toluene (4 mL) and water (1 mL) (previously purged with nitrogen for 20 min) to give a light yellow suspension. The vial's atmosphere was replaced with nitrogen, sealed, heated in dry block at 100° C. for 3.5 h, and cooled to room temperature overnight. The reaction was filtered through celite, rinsed with EtOAc, concentrated, dissolved in minimal DCM, and purified by flash chromatography (silica gel, 0% to 100% EtOAc in hexanes). Appropriate fractions combined, concentrated, and dried from DCM/hexanes yielding (3-biphenyl-4-yl-5-methyl-3H-[1,2,3]triazol-4-yl)-carbamic acid (R)-1-phenyl-ethyl ester (12.4 mg, 66.1% yield) as a white solid. LC/MS calcd. for C₂₄H₂₂N₄O₂ (m/e) 398, obsd. 399 (M+H, ES⁺). ¹H NMR (DMSO-d₆) δ: 9.67 (br. s., 1H), 7.86 (d, J=7.5 Hz, 2H), 7.76 (d, J=7.3 Hz, 2H), 7.49-7.68 (m, 4H), 7.41-7.49 (m, 1H), 6.92-7.40 (m, 5H), 5.70 (br. s., 1H), 2.18 (s, 3H), 1.49 (br. s., 3H).

### Example 28

### [3-(4′-Cyano-biphenyl-4-yl)-5-methyl-3H-[1,2,3]triazol-4-yl]-carbamic acid (R)-1-phenyl-ethyl ester

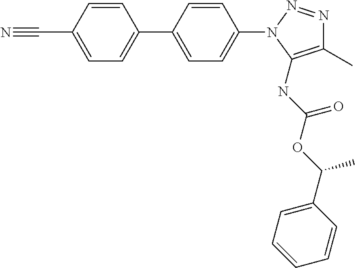 [SMILES: Cc1nnn(-c2ccc(-c3ccc(C#N)cc3)cc2)c1NC(=O)O[C@H](C)c1ccccc1]

In a 20 mL vial, 4-cyanophenylboronic acid (20.1 mg, 0.137 mmol), [3-(4-bromo-phenyl)-5-methyl-3H-[1,2,3]triazol-4-yl]-carbamic acid tert-butyl ester (50 mg, 0.125 mmol), 2-dicyclohexyphosphino-2′,6′-dimethoxybiphenyl (SPhos) (15.3 mg, 0.0374 mmol), tripotassium phosphate (79.4 mg, 0.374 mmol), and Pd(OAc)₂ (4.2 mg, 0.0187 mmol) were combined with toluene (2 mL) and water (0.5 mL) (previously purged with nitrogen for 20 min) to give a light yellow suspension. The vial's atmosphere was replaced with nitrogen, sealed, heated in dry block at 100° C. for 4 h, and cooled to room temperature overnight. The reaction was filtered and rinsed with water (5 mL) and EtOAc (60 mL). The filtrated was washed with water (50 mL) and brine (50 mL). The aqueous layer was extracted with EtOAc (60 ml). The organic layer washed with same brine. The organic layers were combined, concentrated, dissolved in minimal DCM, and purified by flash chromatography (silica gel, 0% to 50% EtOAc in hexanes). Appropriate fractions combined, concentrated, and dried from DCM/hexanes yielding [3-(4′-cyano-biphenyl-4-yl)-5-methyl-3H-[1,2,3]triazol-4-yl]-carbamic acid (R)-1-phenyl-ethyl ester (20 mg, 0.047 mmol, 38% yield) as a white solid. LC/MS calcd. for C₂₅H₂₁N₄O₂ (m/e) 423, obsd. 424 (M+H, ES⁺). ¹H NMR (DMSO-d₆) δ: 9.72 (br. s., 1H), 7.86-8.07 (m, 6H), 7.65 (d, J=8.3 Hz, 2H), 6.80-7.48 (m, 5H), 5.69 (br. s., 1H), 2.18 (s, 3H), 1.48 (br. s., 3H).

### Example 29

### (R)-1-Phenyl-ethyl-1-(4′-(1-(1H-tetrazol-5-yl)cyclopropyl)biphenyl-4-yl)-4-methyl-1H-1,2,3-triazol-5-ylcarbamate

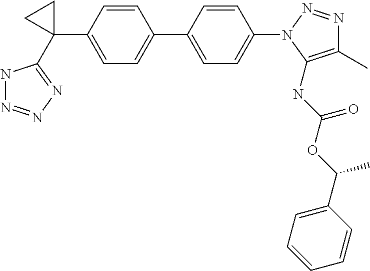 [SMILES: Cc1nnn(-c2ccc(-c3ccc(C4(c5nnn[nH]5)CC4)cc3)cc2)c1NC(=O)O[C@H](C)c1ccccc1]

### Step 1: (R)-1-Phenyl-ethyl-1-(4′-(1-cyanocyclopropyl)biphenyl-4-yl)-4-methyl-1H-1,2,3-triazol-5-ylcarbamate

To a mixture of (R)-1-phenylethyl 4-methyl-1-(4-(4,4,5,5-tetramethyl-1,3,2-dioxaborolan-2-yl)phenyl)-1H-1,2,3-triazol-5-ylcarbamate (485 mg, 1.08 mmol), 1-(4-bromophenyl)cyclopropanecarbonitrile (360 mg, 1.62 mmol), palladium(II) acetate (36.4 mg, 0.16 mmol), 2-dicyclohexylphosphino-2′,6′-dimethoxybiphenyl (133 mg, 0.33 mmol), and potassium phosphate tribasic (689 mg, 3.25 mmol) in a vial were added toluene (9 mL) and water (2.0 mL) at room temperature under nitrogen atmosphere. Then, the cap was closed and the resulting light brown suspension was heated to 105° C. and stirred for 3 h by which time TLC analysis indicated the presence of new spots. Then, the reaction mixture was cooled and it was diluted with water. The organic compound was extracted into EA (2×50 mL) and the combined extracts were washed with brine solution and dried over anhydrous MgSO₄. Filtration and concentration gave the crude residue which was purified by using an ISCO (80 g) column chromatography eluting with 0-100% EA in hexanes. The desired fractions were combined and the solvent was removed under vacuum to isolate (R)-1-phenyl-ethyl-(4′-(1-cyanocyclopropyl)biphenyl-4-yl)-4-methyl-1H-1,2,3-triazol-5-ylcarbamate (190 mg, 38% yield) as a white solid. LC/MS calcd. for C₂₈H₂₅N₅O₂ (m/e) 463, obsd. 464.8 [M+H, ES⁺].

### Step 2: (R)-1-Phenyl-ethyl-1-(4′-(1-(1H-tetrazol-5-yl)cyclopropyl)biphenyl-4-yl)-4-methyl-1H-1,2,3-triazol-5-ylcarbamate

To a solution of (R)-1-phenylethyl 1-(4′-(1-cyanocyclopropyl)biphenyl-4-yl)-4-methyl-1H-1,2,3-triazol-5-ylcarbamate (50 mg, 0.11 mmol) in toluene (5 mL) were added di-n-butyltin oxide (5.37 mg, 0.22 mmol) and azidotrimethylsilane (12.4 mg, 14.3 μL, 0.11 mol) at room temperature under nitrogen atmosphere. The resulting cloudy solution was heated to 100° C. and stirred for 15 h by which time LCMS and TLC analysis indicated the absence of starting material. Then, it was cooled to room temperature and poured into brine solution and EA. The two layers were separated and the aqueous layer was extracted with EA one more time. The combined extracts were washed with brine solution and dried over anhydrous MgSO₄. Filtration and concentration gave the crude product which was purified using an ISCO (40 g) column chromatography eluting with 0-100% EA in hexanes and 10% methanol in dichloromethane. The desired product came with 10% methanol in dichloromethane and the fractions were combined and the solvent was removed under vacuum to obtain (R)-1-phenyl-ethyl-(4′-(1-(1H-tetrazol-5-yl)cyclopropyl)biphenyl-4-yl)-4-methyl-1H-1,2,3-triazol-5-ylcarbamate as a white solid (25 mg, 46% yield). LC/MS calcd. for C₂₈H₂₆N₈O₂ (m/e) 506, obsd. 507.1 [M+H, ES⁺]. ¹H NMR (DMSO-d₆) δ: 16.08 (br. s., 1H), 9.20-9.84 (m, 1H), 7.85 (d, J=7.0 Hz, 2H), 7.73 (d, J=8.3 Hz, 2H), 7.52-7.65 (m, 2H), 7.46 (d, J=8.3 Hz, 2H), 7.34 (br. s., 5H), 5.52-5.84 (m, 1H), 2.17 (s, 3H), 1.51-1.63 (m, 4H), 1.15-1.35 (m, 3H).

### Example 30

### {3-[4′-(1-Methanesulfonylaminocarbonyl-cyclopropyl)-biphenyl-4-yl]-5-methyl-3H[1,2,3]triazol-4-yl}-carbamic acid (R)-1-phenyl-ethyl ester

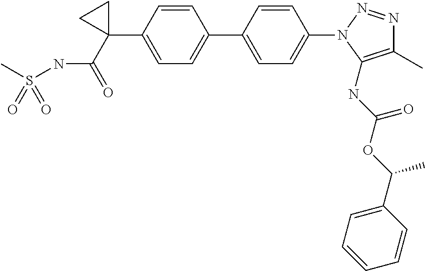 [SMILES: Cc1nnn(-c2ccc(-c3ccc(C4(C(=O)NS(C)(=O)=O)CC4)cc3)cc2)c1NC(=O)O[C@H](C)c1ccccc1]

In a 50 mL round-bottomed flask, 1-{4′-[4-methyl-5-((R)-1-phenyl-ethoxycarbonylamino)[1,2,3]triazol-1-yl]-biphenyl-4-yl}-cyclopropanecarboxylic acid (50 mg, 0.201 mmol) was combined with DCM (1 ml) and DMF (drop) under nitrogen to give a white suspension. To this was added oxalyl chloride (26.3 mg, 18.1 μl, 0.207 mmol) drop wise in two portions with 10 min in between additions. The reaction was stirred at room temperature for 1.5 h. The reaction was concentrated, dried from DCM/Toluene and DCM/hexanes, and dissolved in THF (1.00 ml). In a 5 mL vial, methanesulfonamide (29.6 mg, 311 μmol) was combined with DCM (1 ml) under nitrogen to give a colorless solution. To this was added NaH (60% dispersion in mineral oil, 7.46 mg, 0.311 mmol) and the white suspension was stirred at room temperature for 1.5 h. The acyl chloride THF solution (with rinsed with THF, 1×1 mL) was added drop wise to the sulfonamide mixture. The reaction was stirred at room temperature for 1 day. The reaction was stored in the refrigerator for 3 days. Additional NaH (60% dispersion in mineral oil, 7.46 mg, 0.311 mmol) was added and the reaction was stirred at room temperature for 1 day. Additional NaH (60% dispersion in mineral oil, 7.46 mg, 0.311 mmol) was added and the reaction was stirred at room temperature for 1 day. The reaction was diluted with EtOAc and wash with water and brine, dried over MgSO₄, filtered, concentrated, dissolved in minimal DCM, and purified by flash chromatography (silica gel, 0% to 5% MeOH in DCM). Appropriate fractions combined, concentrated, and dried from DCM/hexanes yielding {3-[4′-(1-methanesulfonylaminocarbonyl-cyclopropyl)-biphenyl-4-yl]-5-methyl-3H-[1,2,3]triazol-4-yl}-carbamic acid (R)-1-phenyl-ethyl ester (5.6 mg, 9.7% yield) as a white solid. LC/MS calcd. for C₂₉H₂₉N₅O₅S (m/e) 559, obsd. 560 (M+H, ES⁺). ¹H NMR (DMSO-d₆) δ: 11.24 (br. s., 1H), 9.68 (br. s., 1H), 7.85 (d, J=7.0 Hz, 2H), 7.72 (d, J=8.3 Hz, 2H), 7.59 (d, J=7.8 Hz, 2H), 7.45 (d, J=8.3 Hz, 2H), 7.34 (br. s., 5H), 5.70 (br. s., 1H), 3.23 (s, 3H), 2.17 (s, 3H), 1.29-1.73 (m, 5H), 1.23 (br. s., 2H).

### Example 31

### 1-{4′-[3-((R)-1-Phenyl-ethoxycarbonylamino)-[1,2,4]triazol-4-yl]-biphenyl-4-yl}-cyclopropanecarboxylic acid

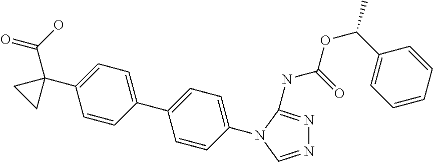 [SMILES: C[C@@H](OC(=O)Nc1nncn1-c1ccc(-c2ccc(C3(C(=O)O)CC3)cc2)cc1)c1ccccc1]

### Step 1: 1-Bromo-4-isothiocyanatobenzene

In a 250 mL round-bottomed flask, calcium carbonate (6.11 g, 61.0 mmol, Eq: 2.1) and 4-bromoaniline (5 g, 29.1 mmol) were combined with dichloromethane (25 ml) and water (25.0 ml) to give a light brown suspension. The reaction mixture was cooled to 0° C. and thiophosgene (3.68 g, 2.45 ml, 32.0 mmol, Eq: 1.1) was added dropwise over 4 min. The reaction was stirred at 0° C. for 30 min then at 25° C. for 19 h. The reaction mixture was filtered through celite and the filter cake was washed with dichloromethane. The aqueous layer was back-extracted with dichloromethane (1×25 mL). The organic layers were combined, washed with H₂O (1×25 mL), saturated NaCl (1×20 mL), dried over Na₂SO₄ and concentrated in vacuo. The light brown solid was dried under vacuum to afford 5.43 g (87%) of the desired product. ¹H NMR (DMSO-d₆) δ ppm 7.55-7.74 (m, 2H), 7.28-7.50 (m, 2H).

### Step 2: (4-Bromophenyl)-thiourea

In a 500 mL round-bottomed flask, 1-bromo-4-isothiocyanatobenzene (1.5 g, 7.01 mmol) was combined with 0.4M ammonia in THF (52.5 mL, 21.0 mmol, Eq: 3) to give a yellow solution. The reaction was stirred at 25° C. overnight. The crude reaction mixture was concentrated in vacuo to afford the desired product as a light brown solid. (M+H)⁺=230.9/233.0 (m/e).

### Step 3: N-(4-Bromophenyl)-hydrazinecarboximidamide nitrate

In a 250 mL round-bottomed flask, 1-(4-bromophenyl)thiourea (1.62 g, 7.01 mmol) was combined with methanol (50 ml) to give a light brown suspension. MeI (1.09 g, 482 μl, 7.71 mmol, Eq: 1.1) was added and the reaction mixture was stirred at 25° C. for 17 h. The crude reaction mixture was concentrated in vacuo to yield a light brown powder. The material was used without further purification.

In a 250 mL round-bottomed flask, 1-(4-bromophenyl)-2-methyl-isothiourea hydroiodide (2.61 g, 7.00 mmol) was combined with water (10 mL) and ethanol (10.0 mL) to give a light brown solution. Hydrazine monohydrate (525 mg, 509 μL, 10.5 mmol, Eq: 1.5) was added and the reaction was stirred at 25° C. for 20 h. The crude reaction mixture was concentrated in vacuo to about half volume and silver nitrate (1.19 g, 7.00 mmol) was added with vigorous stirring. The gray/brown solid was filtered through Celite and the filter cake was washed twice with boiling water. The filtrate was concentrated in vacuo to give a thick yellow oil. The oil was dried under vacuum with slight heating to afford 2.27 g (111%) of the desired material. The product was used without further purification. (M+H)⁺=229.1/231.0 (m/e)

### Step 4: 4-(4-Bromophenyl)-4H-[1,2,4]triazol-3-ylamine

In a 500 mL round-bottomed flask, N′-(4-bromophenyl)-hydrazinecarboximidamide nitrate (2.27 g, 7.77 mmol) and formic acid (715 mg, 596 μL, 15.5 mmol, Eq: 2) were combined to give a yellow solution. The reaction mixture was heated to 120° C. for 3.5 h. The reaction was cooled and basified with 3M NaOH. The mixture was diluted with 150 ml dichloromethane and stirred vigorously. The insoluble solid was filtered and the phases were separated. The organic phase was dried over Na₂SO₄ and filtered. The aqueous phase was discarded. The filtered solid was combined with the dried organic phase and concentrated in vacuo. The residue was taken up in refluxing ethanol and filtered hot to remove a small amount of white insoluble solid. The light brown filtrate was stripped to a tan powder and dried under vacuum to afford 1.665 g (90%) of the desired material. (M+H)⁺=239.0/240.9 (m/e). ¹H NMR (DMSO-d₆) δ ppm 8.20 (s, 1H), 7.66-7.81 (m, 2H), 7.34-7.54 (m, 2H), 5.86 (s, 2H).

### Step 5: 1-[4′-(3-Amino-[1,2,4]triazol-4-yl)-biphenyl-4-yl]-cyclopropanecarboxylic acid methyl ester

In a 20 mL sealed tube, 4-(4-bromophenyl)-4H-1,2,4-triazol-3-amine (349 mg, 1.46 mmol), 4-(1-(methoxycarbonyl)cyclopropyl)phenylboronic acid (450 mg, 2.04 mmol, Eq: 1.4) and 2M Na₂CO₃ (2.19 ml, 4.38 mmol, Eq: 3) were combined with dioxane (6 ml) to give a light yellow suspension. PdCl₂(dppf) (95.4 mg, 117 μmol, Eq: 0.08) was added and the reaction was purged with argon. The reaction mixture was sealed and heated to 100° C. for 24 h under argon. The reaction was cooled and diluted with EtOAc and water. The mixture was filtered and the filtrate was washed with water and brine. The organic layer was dried over Na₂SO₄, combined with the filtered solid and concentrated in vacuo. Celite was added to the residue and the mixture was triturated with refluxing methanol. The mixture was filtered and the filter cake was washed twice with refluxing methanol. The filtrate was stripped in vacuo and the crude material was purified by flash chromatography (silica gel, 80 g, 0% to 10% methanol in dichloromethane) to afford 257 mg (53%) of the desired product as a light brown powder. (M+H)⁺=335.1 (m/e). ¹H NMR (DMSO-d₆) δ ppm 8.24 (s, 1H), 7.79-7.86 (m, 2H), 7.62-7.70 (m, 2H), 7.53-7.60 (m, 2H), 7.41-7.49 (m, 2H), 5.86 (s, 2H), 3.58 (s, 3H), 1.42-1.61 (m, 2H), 1.16-1.35 (m, 2H).

### Step 6: (R)-1-Phenylethyl 1H-imidazole-1-carboxylate

In a 250 mL round-bottomed flask, (R)-1-phenylethanol (2.01 g, 16.5 mmol) and carbonyl diimidazole (2.67 g, 16.5 mmol, Eq: 1.00) were combined with ethyl acetate (40 ml) to give a colorless solution. The reaction mixture was refluxed for 20 h under argon, cooled and diluted with EtOAc. The mixture was washed with H₂O (2×40 mL), saturated NaCl (1×20 mL), dried over Na₂SO₄ and concentrated in vacuo. The material crystallized upon standing to afford 3.42 g (96%) of the desired product as off white needles. ¹H NMR (DMSO-d₆) δ ppm 8.42 (s, 1H), 7.65 (dd, J=1.8, 1.3 Hz, 1H), 7.45-7.54 (m, 2H), 7.22-7.45 (m, 3H), 7.09 (dd, J=1.6, 0.9 Hz, 1H), 6.05 (q, J=6.6 Hz, 1H), 1.66 (d, J=6.6 Hz, 3H).

### Step 7: 1-{4′-[3-((R)-1-Phenyl-ethoxycarbonylamino)-)-[1,2,4]triazol-4-yl]-biphenyl-4-yl}-cyclopropanecarboxylic acid methyl ester

In a 250 round-bottomed flask, methyl 1-(4′-(3-amino-4H-1,2,4-triazol-4-yl)biphenyl-4-yl)cyclopropanecarboxylate (115 mg, 344 μmol) was combined with THF (6 ml) to give a light brown suspension. 1M LiHMDS in THF (447 μl, 447 μmol, Eq: 1.3) was added and the brown solution was stirred at 25° C. under argon for 15 min. (R)-1-phenylethyl 1H-imidazole-1-carboxylate (112 mg, 516 μmol, Eq: 1.5) was added in 1 ml THF and the reaction mixture was stirred for 15 min at 25° C. The reaction was quenched with water and diluted with 10% methanol in dichloromethane. Na₂SO₄ was added and the mixture was filtered through Celite and the brown filtrate was concentrated in vacuo. The crude material was purified by flash chromatography (silica gel, 24 g, 0% to 10% methanol in dichloromethane) to afford 85 mg (51%) of the desired product as an off white solid. (M+H)⁺=483.1 (m/e). ¹H NMR (DMSO-d₆) δ ppm 10.01 (s, 1H), 8.87 (s, 1H), 7.76-7.93 (m, 2H), 7.58-7.75 (m, 2H), 7.38-7.57 (m, 4H), 7.12-7.38 (m, 5H), 5.62 (d, J=6.8 Hz, 1H), 3.58 (s, 3H), 1.47-1.60 (m, 2H), 1.34 (d, J=5.6 Hz, 2H), 1.15-1.31 (m, 3H).

### Step 8: 1-{4′-[3-((R)-1-Phenyl-ethoxycarbonylamino)-[1,2,4]triazol-4-yl]-biphenyl-4-yl}-cyclopropanecarboxylic acid

In a 250 mL round-bottomed flask, 1-{4′-[3-((R)-1-phenyl-ethoxycarbonylamino)[1,2,4]triazol-4-yl]-biphenyl-4-yl}-cyclopropanecarboxylic acid methyl ester (110 mg, 228 μmol) was combined with tetrahydrofuran (5 mL) and methanol (1 mL) to give a yellow solution. 1M LiOH (2 mL, 2.00 mmol, Eq: 8.77) was added and the reaction was stirred at 25° C. for 17 hrs. The crude reaction mixture was concentrated in vacuo, acidified with 1M HCl and diluted with EtOAc. The phases were separated and the organic layer was washed with H₂O (1×15 mL), saturated NaCl (1×15 mL), dried over Na₂SO₄ and concentrated in vacuo. The crude material was purified by flash chromatography (silica gel, 12 g, 0% to 10% methanol in dichloromethane) to afford 86 mg (80%) of the desired product as a white solid. (M+H)⁺=469.2 (m/e). ¹H NMR (DMSO-d₆) δ ppm 12.39 (br. s., 1H), 10.01 (br. s., 1H), 8.87 (br. s., 1H), 7.81 (d, J=8.3 Hz, 2H), 7.65 (d, J=8.3 Hz, 2H), 7.39-7.59 (m, 4H), 7.10-7.39 (m, 5H), 5.62 (d, J=6.3 Hz, 1H), 1.45-1.54 (m, 2H), 1.40 (br. s., 1H), 1.09-1.37 (m, 4H).

### Examples 32 and 33

### (R)-1-(4′-(4-Methyl-5-((1-phenylethoxy)carbonylamino)-1H-1,2,3-triazol-1-yl)biphenyl-4-yl)cyclobutanecarboxylic acid (Example 32)

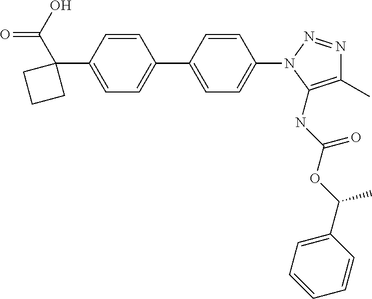 [SMILES: Cc1nnn(-c2ccc(-c3ccc(C4(C(=O)O)CCC4)cc3)cc2)c1NC(=O)O[C@H](C)c1ccccc1]

### (R)-2-{4′-[4-Methyl-5-(-1-phenylethoxycarbonylamino)-[1,2,3]triazol-1-yl]-biphenyl-4-yl}-pent-4-enoic acid (Example 33)

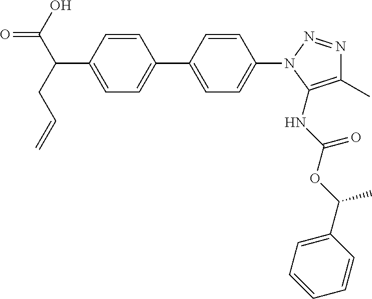 [SMILES: C=CCC(C(=O)O)c1ccc(-c2ccc(-n3nnc(C)c3NC(=O)O[C@H](C)c3ccccc3)cc2)cc1]

### Step 1: 1-(4-Bromophenyl)cyclobutane carboxylic acid ethyl ester and 2-(4-bromophenyl)-pent-4-enoic acid ethyl ester

To a solution of 2-(4-bromophenyl)acetic acid ethyl ester (5.98 g, 24.6 mmol) in DMF (60 mL) was cooled to 0° C. and then the solid sodium hydride (2.17 g, 54.4 mmol) was added in five portions in a period of 10 minutes. During the addition, it was a vigorous reaction with foaming and the reaction mixture was turned to yellow suspension. Additional 10 mL of DMF was used to wash the sodium hydride. The resulting yellow suspension was stirred for 20 minutes and the neat 1,3-dibromopropane (5.46 g, 2.75 mL, 27.1 mmol) was added at this temperature. After 5 minutes, the cooling bath was removed and the reaction mixture was allowed to warm to room temperature. During this period, the reaction mixture was turned to a colorless cloudy solution and it was stirred for 1 h. Then, the reaction mixture was poured into a 0.1 N HCl and the organic compound was extracted into EA (2×100 mL). The combined extracts were washed with water and brine solution and dried over anhydrous MgSO₄. Filtration of the drying agent and concentration of the filtrate gave the crude white suspension which was purified using an ISCO (120 g) column chromatography eluting with EA in hexanes (0-15%). Both compounds, 1-(4-bromophenyl)cyclobutane carboxylic acid ethyl ester and 2-(4-bromophenyl)-pent-4-enoic acid ethyl ester, were isolated as a mixture.

### Step 2: (R)-1-(4′-(4-Methyl-5-((1-phenylethoxy)carbonylamino)-1H-1,2,3-triazol-1-yl)biphenyl-4-yl)cyclobutanecarboxylic acid ethyl ester and (R)-2-{4′-[4-methyl-5-(1-phenyl-ethoxycarbonylamino)-[1,2,3]triazol-1-yl]-biphenyl-4-yl}-pent-4-enoic acid ethyl ester

To a suspension of (R)-1-phenylethyl 4-methyl-1-(4-(4,4,5,5-tetramethyl-1,3,2-dioxaborolan-2-yl)phenyl)-1H-1,2,3-triazol-5-ylcarbamate (1.34 g, 3 mmol), 1-(4-bromophenyl)cyclobutane carboxylic acid ethyl ester and 2-(4-bromophenyl)-pent-4-enoic acid ethyl ester (1.02 g, 3.6 mmol), palladium(II) acetate (135 mg, 0.6 mmol), 2-dicyclohexylphosphino-2′,6′-dimethoxybiphenyl (493 mg, 1.2 mmol), and potassium phosphate tribasic (1.91 g, 9.0 mmol) in a 100 mL RB flask were added toluene (18 mL) and water (4.0 mL) at room temperature under nitrogen atmosphere. Then, the resulting light brown suspension was heated to 105° C. and stirred for 3 h by which time TLC analysis indicated the absence of starting material. Then, the black reaction mixture was cooled to room temperature and diluted with water. The organic compound was extracted into EA (2×100 mL) and the combined extracts were washed with brine solution and dried over anhydrous MgSO₄. Filtration of the drying agent and concentration of the filtrate gave the crude residue which was purified by using an ISCO (120 g) column chromatography eluting with EA in hexanes (0-100%) to obtain (R)-1-(4′-(4-methyl-5-((1-phenylethoxy)carbonylamino)-1H-1,2,3-triazol-1-yl)biphenyl-4-yl)cyclobutanecarboxylic acid ethyl ester and (R)-2-{4′-[4-methyl-5-(1-phenyl-ethoxycarbonylamino)-[1,2,3]triazol-1 yl]-biphenyl-4-yl}-pent-4-enoic acid ethyl ester as a mixture. LC/MS calcd. for C₃₁H₃₂N₄O₄ (m/e) 524, obsd. 525.3 [M+H, ES⁺].

### Step 3: ((R)-1-(4′-(4-Methyl-5-((1-phenylethoxy)carbonylamino)-1H-1,2,3-triazol-1-yl)biphenyl-4-yl)cyclobutanecarboxylic acid and (R)-2-{4′-[4-methyl-5-(1-phenyl-ethoxycarbonylamino)[1,2,3]triazol-1-yl]-biphenyl-4-yl}-pent-4-enoic acid

To a solution of a mixture of obtained (R)-1-(4′-(4-methyl-5-((1-phenylethoxy)carbonylamino)-1H-1,2,3-triazol-1-yl)biphenyl-4-yl)cyclobutanecarboxylic acid ethyl ester and (R)-2-{4′-[4-methyl-5-(1-phenyl-ethoxycarbonylamino)-[1,2,3]triazol-1-yl]-biphenyl-4-yl}-pent-4-enoic acid ethyl ester (120 mg, 0.229 mmol) in THF (6.0 mL) and EtOH (6.0 mL) was added an excess of 1 N sodium hydroxide (2.29 mL, 2.29 mmol) solution in water at room temperature. The resulting light yellow solution was stirred for 2 days at room temperature at which time TLC analysis indicated the absence of starting material. Then, the solvent was removed under vacuum and the basic aqueous layer was neutralized with 1 N HCl. The resulting white cloudy solution was extracted with EA (2×50 mL) and the combined extracts were washed with brine solution. Dried and removed the solvent to afford the crude mixture which was purified using DAICEL OI column (3×25 cm, 40% methanol and CO₂, 70 mL/min and the peaks were collected at 220 nM. Peak 1 was collected and the solvent was removed to obtain (R)-2-{4′-[4-methyl-5-(1-phenyl-ethoxycarbonylamino)-[1,2,3]triazol-1-yl]-biphenyl-4-yl}-pent-4-enoic acid (12 mg, 10.5% yield, Example 33). ¹H NMR (CHLOROFORM-d) δ: 7.29-7.61 (m, 8H), 7.19 (s, 6H), 5.44-5.83 (m, 2H), 4.92-5.14 (m, 2H), 3.67 (t, J=6.4 Hz, 1H), 2.81 (dt, J=14.2, 7.2 Hz, 1H), 2.41-2.60 (m, 1H), 2.24 (s, 3H), 1.12-1.30 (m, 3H). And the peak 2 was collected and the solvent was removed to obtain ((R)-1-(4′-(4-methyl-5-((1-phenylethoxy)carbonylamino)-1H-1,2,3-triazol-1-yl)biphenyl-4-yl)cyclobutanecarboxylic acid (26 mg, 23% yield, Example 32). ¹H NMR (CHLOROFORM-d) δ: 7.29-7.55 (m, 8H), 7.19 (s, 6H), 5.69 (br. s., 1H), 2.78-2.91 (m, 2H), 2.45-2.59 (m, 2H), 2.24 (s, 3H), 1.99-2.11 (m, 1H), 1.87 (td, J=10.0, 4.5 Hz, 1H), 1.10-1.35 (m, 3H). LC/MS calcd. for C₂₉H₂₈N₄O₄ (m/e) 496, obsd. 497.3 [M+H, ES⁺].

### Example 34

### (R)-2-(4-(4-(4-Methyl-5-((1-phenylethoxy)carbonylamino)-1H-1,2,3-triazol-1-yl)phenyl)cyclohexyl)acetic acid

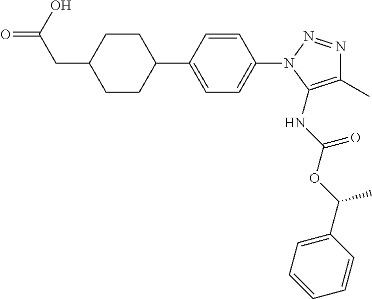 [SMILES: Cc1nnn(-c2ccc(C3CCC(CC(=O)O)CC3)cc2)c1NC(=O)O[C@H](C)c1ccccc1]

### Step 1: 2-(4-Idocyclohexyl)-acetic acid ethyl ester

To a mixture of ethyl 2-(4-hydroxycyclohexyl)acetate (3 g, 16.1 mmol), iodine (6.13 g, 24.2 mmol), imidazole (1.64 g, 24.2 mmol), and triphenylphosphine (6.34 g, 24.2 mmol) was added dichloromethane (100 mL) at room temperature under nitrogen atmosphere. The resulting brown suspension was stirred for 15 h at which time TLC analysis indicated the absence of starting material. Then, the solvent was removed under vacuum and most of the residue was dissolved in EA (˜500 mL) and some of the residue was not dissolved which was found to be Ph₃P═O by ¹H NMR. The EA solution was washed two times with a solution of water and methanol (3:1) to remove the remaining triphenylphosphineoxide and then washed with brine solution. The organic layer was dried over anhydrous MgSO₄, filtration, and concentration gave the crude residue which was purified using an ISCO (120 g) column chromatography eluting with EA in hexanes (0-50%). The desired fractions were combined and the solvent was removed under vacuum to obtain 2-(4-iodocyclohexyl)acetic acid ethyl ester (3.39 g, 71.1% yield) as a viscous light yellow oil. 1H NMR of this product indicated that it contained ˜30-40% of elimination side product (olefin) and it was not separable on TLC.

### Step 2: 1-[4-(4-(2-Ethoxy-2-oxoethyl)cyclohexyl)phenyl)-5-methyl-1H-1,2,3-triazole-carboxylic acid tert-butyl ester

In a 3-neck 50 mL RB flask, equipped with an additional funnel and a thermometer, was charged with zinc dust, 99.9% (490 mg, 7.5 mmol) at room temperature under nitrogen atmosphere. Then, the flask was purged with nitrogen under vacuum and THF (2 mL) was added to cover the zinc dust. 1,2-Dibromoethane (60.6 mg, 27.8 μL, 0.322 mmol) was added and the mixture was heated with heat gun until evolution of ethylene gas ceased. Then, the suspension was cooled to room temperature and chlorotrimethylsilane (35.0 mg, 40.8 μL, 0.322 mmol) was added and the mixture was stirred for 15 min at room temperature. Then, a solution of 2-(4-iodocyclohexyl)acetic acid ethyl ester (740 mg, 2.5 mmol) in THF (2 mL and 1 mL for washing) was added drop-wise for 5 minutes. After addition, the reaction mixture was heated to ˜60° C. with oil bath and stirred for 3 h by which time TLC analysis of the hydrolyzed reaction mixture indicated the absence of starting material. Then, the heating was stopped and the excess zinc dust was allowed to settle (15 h) to give a top layer as a colorless solution.

In another 2-neck 25 mL RB flask, palladium(II) acetate (24.9 mg, 0.111 mmol) and 2-dicyclohexylphosphino-2′,6′-dimethoxybiphenyl (91.0 mg, 0.222 mmol) were charged and the flask was purged with nitrogen gas. Then, THF (1 mL) was added and the resulting light brown suspension was stirred for 5 min before the addition of a solution of 1-(4-bromophenyl)-4-methyl-1H-1,2,3-triazole-5-carboxylic acid tert-butyl ester (150 mg, 0.444 mmol) in THF (3 mL) at room temperature under nitrogen atmosphere. Then, the above prepared colorless zinc solution was added to this mixture. After the addition, it turned to a dark brown solution which was then heated to 60° C. and stirred for 8 h at which time TLC analysis of the hydrolyzed reaction mixture indicated the absence of starting material. Then, it was cooled to room temperature and diluted with saturated ammonium chloride solution and EA. The two layers were separated and the aqueous layer was extracted with EA. The combined organic extracts were washed with brine solution and dried over anhydrous MgSO₄. Filtration of the drying agent and concentration of the filtrate gave the crude light yellow residue which was purified using an ISCO (80 g) column eluting with EA in hexanes (0-60%). The desired fractions were combined and the solvent was removed under vacuum to obtain 1-[4-(4-(2-ethoxy-2-oxoethyl)cyclohexyl)phenyl)-5-methyl-1H-1,2,3-triazole-carboxylic acid tert-butyl ester (55 mg, 29% yield) as a light brown oil. LC/MS calcd. for C₂₄H₃₃N₃O₄ (m/e) 427, obsd. 428.1 [M+H, ES⁺].

### Step 3: 1-[4-(4-(2-Ethoxy-2-oxoethyl)cyclohexyl)phenyl)-5-methyl-1H-1,2,3-triazole-carboxylic acid

To a light yellow solution of 1-(4-(4-(2-ethoxy-2-oxoethyl)cyclohexyl)phenyl)-4-methyl-1H-1,2,3-triazole-5-carboxylic acid tert-butyl ester (96 mg, 0.225 mmol) in dichloromethane (5 mL) was added an excess of TFA (2.56 g, 1.73 mL, 22.5 mmol) at room temperature under nitrogen atmosphere. The resulting light yellow solution was stirred for 20 h at which time TLC analysis indicated the absence of starting material. Then, the solvent was removed under vacuum and the residue was azeotrophed with toluene. The residue was dried under high vacuum to obtain 1-[4-(4-(2-ethoxy-2-oxoethyl)cyclohexyl)phenyl)-5-methyl-1H-1,2,3-triazole-carboxylic acid (85 mg, 97% yield) as a light brown solid. LC/MS calcd. for C₂₀H₂₅N₃O₄ (m/e) 371, obsd. 372.1 [M+H, ES⁺].

### Step 4: (R)-2-(4-(4-(4-Methyl-5-((1-phenylethoxy)carbonylamino)-1H-1,2,3-triazol-1-yl)phenyl)cyclohexyl)acetic acid ethyl ester

To a light brown solution with few solids of 1-(4-(4-(2-ethoxy-2-oxoethyl)cyclohexyl)phenyl)-4-methyl-1H-1,2,3-triazole-5-carboxylic acid (85 mg, 0.229 mmol) in toluene (5 mL) was added triethylamine (46.3 mg, 63.8 μL, 0.458 mmol) at room temperature. To the resulting solution were added diphenylphosphoryl azide (69.3 mg, 54.2 μL, 0.252 mmol) followed by (R)-1-phenylethanol (30.8 mg, 30.4 μL, 0.252 mmol) at room temperature. The resulting solution was heated with oil bath to 81° C. and stirred for 1 h at which time TLC analysis indicated the presence of a new spot. Then, the reaction mixture was cooled to room temperature and the solvent was removed under vacuum. The crude residue (˜450 mg) was suspended in dichloromethane and filtered. The filtrate was loaded onto an ISCO (40 g) column chromatography eluting with EA in hexanes (0-100%). The desired fractions were combined and the solvent was removed under vacuum to obtain (R)-2-(4-(4-(4-methyl-5-((1-phenylethoxy)carbonylamino)-1H-1,2,3-triazol-1-yl)phenyl)cyclohexyl)acetic acid ethyl ester (50 mg, 45% yield) as a white solid. LC/MS calcd. for C₂₈H₃₄N₄O₄ (m/e) 490, obsd. 491.3 [M+H, ES⁺].

### Step 5: (R)-2-(4-(4-(4-Methyl-5-((1-phenylethoxy)carbonylamino)-1H-1,2,3-triazol-1-yl)phenyl)cyclohexyl)acetic acid

To a colorless solution of (R)-2-(4-(4-(4-methyl-5-((1-phenylethoxy)carbonylamino)-1H-1,2,3-triazol-1-yl)phenyl)cyclohexyl)acetic acid ethyl ester (46 mg, 0.94 mmol) in THF (5 mL) and EtOH (5 mL) was added an excess of 1 M solution of sodium hydroxide (2.81 mL, 2.81 mmol) in water. The resulting colorless solution was stirred for 15 h at room temperature at which time LC/MS and TLC analysis indicated the absence of starting material. Then, the solvent was removed under vacuum and the basic aqueous layer was neutralized with 1 N HCl. The resulting white solids were collected by filtration and washed with water and hexanes. After air drying, (R)-2-(4-(4-(4-methyl-5-((1-phenylethoxy)carbonylamino)-1H-1,2,3-triazol-1-yl)phenyl)cyclohexyl)acetic acid (35 mg, 80.7% yield) was isolated as a white solid. ¹H NMR (DMSO-d₆) δ: 12.05 (s, 1H), 9.15-9.74 (m, 1H), 6.97-7.64 (m, 9H), 5.70 (br. s., 1H), 2.54-2.72 (m, 1H), 2.07-2.26 (m, 5H), 1.62-1.91 (m, 5H), 1.36-1.59 (m, 4H), 1.04-1.32 (m, 3H). LC/MS calcd. for C₂₆H₃₀N₄O₄ (m/e) 462, obsd. 463.3 [M+H, ES⁺].

### Example 35

### {3-[4′-(1-Methanesulfonylaminocarbonyl-cyclopropyl)-biphenyl-4-yl]-5-methyl-3H[1,2,3]triazol-4-yl}-carbamic acid (R)-1-(3-trifluoromethyl-phenyl)-ethyl ester

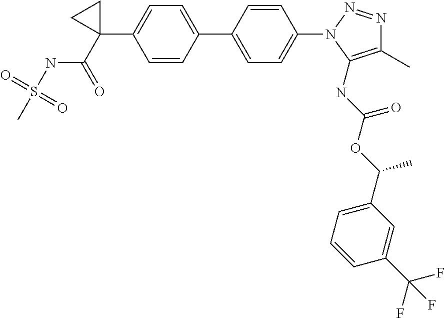 [SMILES: Cc1nnn(-c2ccc(-c3ccc(C4(C(=O)NS(C)(=O)=O)CC4)cc3)cc2)c1NC(=O)O[C@H](C)c1cccc(C(F)(F)F)c1]

### Step 1: N-[1-(4-Bromo-phenyl)-cyclopropanecarbonyl]-methanesulfonamide

In a 100 mL round-bottomed flask, 1-(4-bromo-phenyl)-cyclopropanecarboxylic acid (4 g, 16.6 mmol) was combined with DCM (15 mL) and 3 drops of DMF to give a white suspension. To this was added drop wise a clear solution of oxalyl chloride (6.96 g, 4.8 mL, 54.8 mmol) dissolved in DCM (6 mL). After 10 min, the mixture became clear and the reaction was stirred at room temperature for 2 hr. The reaction was concentrated, dried from toluene and hexanes, and stored in a freezer overnight. In a 200 mL round-bottomed flask, NaH (60% mineral dispersion, 876 mg, 36.5 mmol) was washed with hexanes and the resulting solid was diluted with DMF (6 mL) to give a white suspension. The suspension was cooled in an ice bath and methanesulfonamide (3.16 g, 33.2 mmol) dissolved in DMF (6 mL) was added drop wise under nitrogen. After addition (5 min) the ice bath was removed and the reaction was warmed to room temperature overnight. The reaction was cooled in an ice bath, the acid chloride previous prepared and dissolved in DMF (6 mL) was added drop wise, and the reaction was warmed to room temperature overnight. The reaction was diluted with 0.2 N HCl (200 mL) and extracted with EtOAc (2×100 mL). The organic layers were washed with brine, combined, dried, over MgSO₄, and concentrated. The crude material was dissolved in minimal DCM and purified by flash chromatography (silica gel, 0% to 60% EtOAc in hexanes, 0.5% AcOH). The appropriate fractions were combined, concentrated, and dried from DCM/hexanes yielding N-[1-(4-bromo-phenyl)-cyclopropanecarbonyl]-methanesulfonamide (2.74 g, 51.9% yield), as a white solid. LC/MS calcd. for C₁₁H₁₂BrNO₃S (m/e) 317/319, obsd. 318/320 (M+H, ES⁺).

### Step 2: N-{1-[4-(4,4,5,5-Tetramethyl-[1,3,2]dioxaborolan-2-yl)-phenyl]-cyclopropanecarbonyl}-methanesulfonamide

In a 350 mL reaction vial containing N-[1-(4-bromo-phenyl)-cyclopropanecarbonyl]-methanesulfonamide (2.71 g, 8.52 mmol) was added bis-pinacolatodiboron (3.24 g, 12.8 mmol) and potassium acetate (2.51 g, 25.6 mmol, Eq: 3) and 1,4 dioxane (63.8 mL) to give a white suspension. The mixture was purged with nitrogen for 20 min and then PdCl₂(dppf)CH₂Cl₂ (701 mg, 859 μmol) was added. The vial was sealed and heated in an oil bath at 80° C. for 16 hr. The reaction was diluted with EtOAc (150 mL), filtered, rinsed with 0.2 M HCl (200 mL) and EtOAc (50 mL). The combined filtrate was mixed vigorously, filtered, and separated. The aqueous layer was extracted once with EtOAc (150 mL). The organic layers were washed with brine, combined, dried over MgSO₄, filtered, concentrated, and dried from DCM/hexanes as a brown solid (4 g). The crude material was supported on Celite and purified by flash chromatography (silica gel, 0 to 60% EtOAc in hexanes, 0.5% AcOH). The appropriate fractions were combined, concentrated, and dried from DCM/Hexanes, yielding N-{1-[4-(4,4,5,5-tetramethyl-[1,3,2]dioxaborolan-2-yl)-phenyl]-cyclopropanecarbonyl}-methanesulfonamide (2.75 g, 88.4% yield), as a white solid. LC/MS calcd. for C₁₇H₂₄BNO₅S (m/e) 365, obsd. 366 (M+H, ES⁺).

### Step 3: {3-[4′-(1-Methanesulfonylaminocarbonyl-cyclopropyl)-biphenyl-4-yl]-5-methyl-3H-[1,2,3]triazol-4-yl}-carbamic acid (R)-1-(3-trifluoromethyl-phenyl)-ethyl ester

In a 8 mL vial, [3-(4-bromo-phenyl)-5-methyl-3H-[1,2,3]triazol-4-yl]-carbamic acid (R)-1-(3-trifluoromethyl-phenyl)-ethyl ester (47 mg, 100 μmol), N-{1-[4-(4,4,5,5-tetramethyl-[1,3,2]dioxaborolan-2-yl)-phenyl]-cyclopropanecarbonyl}-methanesulfonamide (40.2 mg, 110 μmol), DPPF (8.33 mg, 15.0 μmol) and PdCl₂(dppf)CH₂Cl₂ (12.3 mg, 15.0 μmol) were combined with DMF (1 mL) (previous purged with nitrogen for 20 min) to give a light brown/red solution. To this was added 2N Na₂CO₃ (200 μL, 401 μmol) (previous purged with nitrogen for 20 min) and a precipitate formed. The resulting red mixture was purged with nitrogen for 1 min. The vial was sealed, placed in a dry block, and heated at 80° C. for 2 hr. The reaction was diluted with EtOAc (50 mL) and 0.1 N HCl (50 mL), mixed, filtered, and separated. The aqueous layer was extracted with EtOAc (50 mL). The organic layers were washed with brine, combined, dried over MgSO₄, filtered, concentrated, and dried from DCM/hexanes as a yellow film (120 mg). The crude material was supported on Celite and purified by flash chromatography (silica gel, 0% to 60% EtOAc in hexanes, 0.5% AcOH). Appropriate fractions were combined, concentrated, dried from DCM/hexanes and DCM yielding {3-[4′-(1-methanesulfonylaminocarbonyl-cyclopropyl)-biphenyl-4-yl]-5-methyl-3H[1,2,3]triazol-4-yl}-carbamic acid (R)-1-(3-trifluoromethyl-phenyl)-ethyl ester (32 mg, 50.9% yield) as a light yellow solid. LC/MS calcd. for C₃₀H₂₈F₃N₅O₅S (m/e) 627, obsd. 628 (M+H, ES⁺). ¹H NMR (DMSO-d₆) δ: 11.23 (br. s., 1H), 9.80 (br. s., 1H), 7.85 (d, J=6.5 Hz, 2H), 7.49-7.77 (m, 8H), 7.45 (d, J=8.3 Hz, 2H), 5.68-5.95 (m, 1H), 3.23 (s, 3H), 2.17 (br. s., 3H), 1.44-1.64 (m, 4H), 1.23 (br. s., 3H).

### Example 36

### Calcium Flux Assay Using Fluorometric Imaging Plate Reader (FLIPR)

Cell Culture Conditions: The ChemiScreen Calcium-optimized stable cell line containing the human recombinant LPA1 Lysophospholipid receptor was purchased from Chemicon International, Inc./Millipore. The cells were cultured in DMEM-high glucose supplemented with 10% fetal bovine serum, 2 mM glutamine, 100 U/mL penicillin/100 μg/mL streptomycin, 1× non-essential amino acids, 10 mM HEPES and 0.25 mg/mL Geneticin. Cells were harvested with trypsin-EDTA and counted using ViaCount reagent. The cell suspension volume was adjusted to 2.0×10⁵ cells/mL with complete growth media. Aliquots of 50 μL were dispensed into 384 well black/clear tissue culture treated plates (BD) and the microplates were placed in a 37° C. incubator overnight. The following day plates were used in the assay.

Dye Loading and Assay: Loading Buffer (FLIPR Calcium-4, Molecular Devices) was prepared by dissolving the contents of one bottle into 100 mL Hank's Balanced Salt Solution containing 20 mM HEPES and 2.5 mM probenecid. Plates were loaded onto Biotek plate washer and growth media was removed and replaced with 20 μL of Hank's Balanced Salt Solution containing 20 mM HEPES and 2.5 mM probenecid, followed by 25 μL of Loading Buffer. The plates were then incubated for 30 minutes at 37° C.

During the incubation, test compounds were prepared by adding 90 μL of HBSS/20 mM HEPES/0.1% BSA buffer to 2 μL of serially diluted compounds. To prepare serial dilutions, 10 mM stocks of compounds were prepared in 100% DMSO. The compound dilution plate was set up as follows: well #1 received 29 μL of stock compound and 31 μL DMSO; wells 2-10 received 40 μL of DMSO; mixed and transferred 20 μL of solution from well #1 into well #2; continued with 1:3 serial dilutions out 10 steps; transferred 2 μL of diluted compound into duplicate wells of 384 well “assay plate” and then added the 90 μL of buffer.

After incubation, both the cell and “assay” plates were brought to the FLIPR and 20 μL of the diluted compounds were transferred to the cell plates by the FLIPR. Compound addition was monitored by FLIPR to detect any agonist activity of the compounds. Plates were then incubated for 30 minutes at room temperature protected from light. After the incubation, plates were returned to the FLIPR and 20 μL of 4.5× concentrated agonist was added to the cell plates. During the assay, fluorescence readings were taken simultaneously from all 384 wells of the cell plate every 1.5 seconds. Five readings were taken to establish a stable baseline, then 20 μL of sample was rapidly (30 μL/sec) and simultaneously added to each well of the cell plate. The fluorescence was continuously monitored before, during and after sample addition for a total elapsed time of 100 seconds. Responses (increase in peak fluorescence) in each well following agonist addition was determined. The initial fluorescence reading from each well, prior to ligand stimulation, was used as zero baseline value for the data from that well. The responses were expressed as % inhibition of the buffer control. The IC₅₀ value, defined as the concentration of a compound required for 50% inhibition of the buffer control, was calculated by fitting the percent inhibition data for 10 concentrations to a sigmoidal dose-response (4 parameter logistic) model using Genedata Condoseo program [model 205, F(x)=(A+(B−A)/(1+((C/x)̂D))))] and the results shown in Table 1 below:

TABLE 1
LPA1 and LPA3 antagonist activities

| | LPA1 IC₅₀(μM) or | LPA3 IC₅₀(μM) or |
|---|---|---|
| Example # | (inhibition % @μM) | (inhibition % @μM) |
| 1 | 0.025 | >30 |
| 2 | >30 (40% @ 30) | >30 |
| 3 | >30 | >30 |
| 4 | >30 | >30 |
| 5 | 0.035 | >30 |
| 6 | 0.112 | 25.9 (55.2% @ 30) |
| 7 | 0.174 | 6.86 |
| 8 | >30 | >30 |
| 9 | 0.217 | >30 |
| 10 | 0.398 | >30 |
| 11 | >30 | >30 |
| 12 | 0.134 | >30 |
| 13 | 0.161 | >30 |
| 14 | 0.985 | >30 |
| 15 | 0.022 | (46.3% @ 30) |
| 16 | 0.245 | >30 |
| 17 | 0.043 | 21.73 (63.7% @ 30) |
| 18 | 1.228 (79.8% @ 30) | >30 |
| 19 | 0.412 | 4.82 |
| 20 | 21.23 (58.3% @ 30) | 14.3 (72.5% @ 30) |
| 21 | 0.036 | >30 (22% @ 30) |
| 22 | >30 | >30 |
| 23 | 0.796 (80.9% @ 30) | >30 |
| 24 | >30 | >30 |
| 25 | >30 | >30 |
| 26 | >30 | >30 |
| 27 | >30 | >30 |
| 28 | >30 | >30 |
| 29 | 0.023 | >30 |
| 30 | 0.033 | >30 |
| 31 | >30 (11% @ 30) | >30 |
| 32 | 0.174 | >30 |
| 33 | 0.088 | >30 |
| 34 | 9.478 | >30 |
| 35 | 4.534 | 5.736 |

It is to be understood that the invention is not limited to the particular embodiments of the invention described above, as variations of the particular embodiments may be made and still fall within the scope of the appended claims.

1-2. (canceled)

3. A compound of formula (I), (Ia), (Ib) or (Ic): 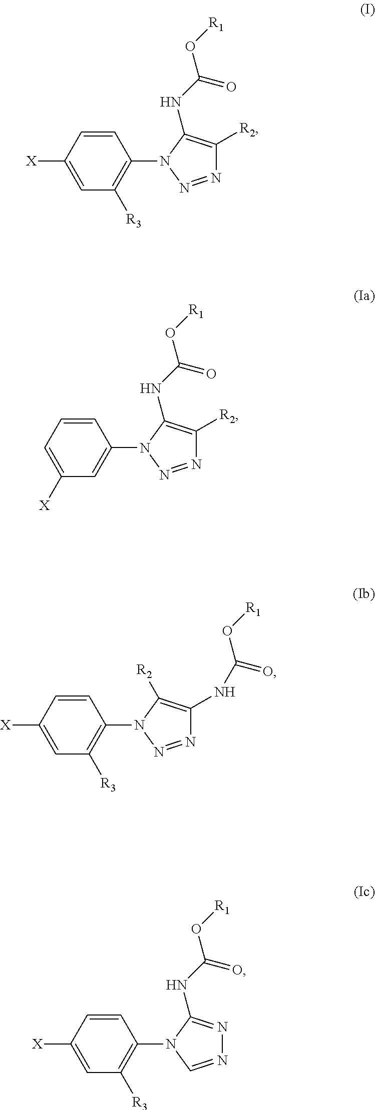
wherein:
R₁ is lower alkyl or indanyl, said lower alkyl being unsubstituted or substituted with cycloalkyl, unsubstituted phenyl or phenyl substituted with halogen or —CF₃;
R₂ is hydrogen or lower alkyl;
R₃ is hydrogen, fluorine or —OCH₃;
X is cycloalkyl acetic acid or 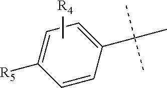
R₄ is hydrogen or halogen;
R₅ is hydrogen, cyano, tetrazole-cyclopropyl, methanesulfonylaminocarbonyl-cyclopropyl or 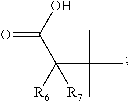
R₆ and R₇ are, independently of each other, hydrogen, lower alkyl or lower alkenyl; or
R₆ and R₇, together with the carbon to which they are attached, form a cycloalkyl group,
or a pharmaceutically acceptable salt thereof.

4. The compound according to claim 3, wherein R₁ is unsubstituted lower alkyl.

5. The compound according to claim 3, wherein R₁ is dimethylpropyl, butyl or isopropyl.

6. The compound according to claim 3, wherein R₁ is lower alkyl substituted with cycloalkyl, unsubstituted phenyl or phenyl substituted with halogen or —CF₃.

7. The compound according to claim 6, wherein R₁ is —CH(CH₃)-phenyl, —CH(CH₃)-fluorophenyl, —CH(CH₃)-trifluoromethylphenyl, ethyl-cyclopropyl or ethyl-cyclobutyl.

8. The compound according to claim 3, wherein R₂ is lower alkyl.

9. The compound according to claim 3, wherein R₂ is methyl.

10. The compound according to claim 3, wherein R₃ is hydrogen.

11. The compound according to claim 3, wherein X is cyclohexyl acetic acid.

12. The compound according to claim 3, wherein X is

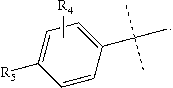

13. The compound according to claim 12, wherein R₄ is hydrogen or fluorine.

14. The compound according to claim 12, wherein R₅ is hydrogen, cyano, tetrazole-cyclopropyl or methanesulfonylaminocarbonyl-cyclopropyl.

15. The compound according to claim 12, wherein R₅ is

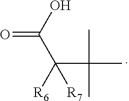

16. The compound according to claim 12, wherein R₆ and R₇ are, independently of each other, hydrogen or methyl.

17. The compound according to claim 12, wherein R₆ and R₇, together with the carbon to which they are attached, form a cyclopropyl group.

18. The compound according to claim 3, wherein R₁ is lower alkyl or indanyl, said lower alkyl being unsubstituted or substituted with cycloalkyl or unsubstituted phenyl; R₂ is hydrogen or lower alkyl; R₃ is hydrogen, fluorine or —OCH₃; X is cycloalkyl acetic acid or 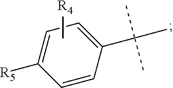
wherein R₄ is hydrogen or halogen and R₅ is hydrogen, cyano, tetrazole-cyclopropyl, methanesulfonylaminocarbonyl-cyclopropyl or 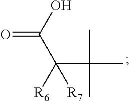
wherein R₆ and R₇ are, independently of each other, hydrogen or lower alkyl; or R₆ and R₇, together with the carbon to which they are attached, form a cycloalkyl group, or a pharmaceutically acceptable salt thereof.

19. The compound according to claim 3, wherein R₁ is lower alkyl being substituted with unsubstituted phenyl; R₂ is hydrogen or lower alkyl; R₃ is hydrogen, fluorine or —OCH₃; X is cycloalkyl acetic acid or 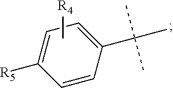
wherein R₄ is hydrogen or halogen and R₅ is hydrogen, cyano, tetrazole-cyclopropyl, methanesulfonylaminocarbonyl-cyclopropyl or 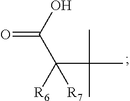
wherein R₆ and R₇ are, independently of each other, hydrogen or lower alkyl; or R₆ and R₇, together with the carbon to which they are attached, form a cycloalkyl group, or a pharmaceutically acceptable salt thereof.

20. The compound according to claim 3, wherein R₁ is lower alkyl or indanyl, said lower alkyl being unsubstituted or substituted with cycloalkyl, unsubstituted phenyl or phenyl substituted with halogen or —CF₃; R₂ is ethyl; R₃ is hydrogen, fluorine or —OCH₃; X is cycloalkyl acetic acid or 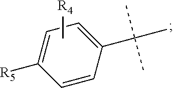
wherein R₄ is hydrogen or halogen and R₅ is hydrogen, cyano, tetrazole-cyclopropyl, methanesulfonylaminocarbonyl-cyclopropyl or 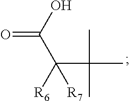
wherein R₆ and R₇ are, independently of each other, hydrogen or lower alkyl; or R₆ and R₇, together with the carbon to which they are attached, form a cycloalkyl group, or a pharmaceutically acceptable salt thereof.

21. The compound according to claim 3, wherein R₁ is lower alkyl or indanyl, said lower alkyl being unsubstituted or substituted with cycloalkyl, unsubstituted phenyl or phenyl substituted with halogen or —CF₃; R₂ is hydrogen or lower alkyl; R₃ is hydrogen, fluorine or —OCH₃; X is cycloalkyl acetic acid or 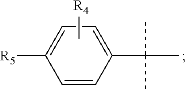
wherein R₄ is hydrogen or halogen and R₅ is 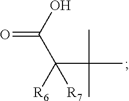
wherein R₆ and R₇, together with the carbon to which they are attached, form a cycloalkyl group, or a pharmaceutically acceptable salt thereof.

22. The compound according to claim 3, wherein R₁ is lower alkyl being substituted with unsubstituted phenyl; R₂ is hydrogen or lower alkyl; R₃ is hydrogen, fluorine or —OCH₃; X is cycloalkyl acetic acid or 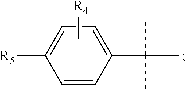
wherein R₄ is hydrogen or halogen and R₅ is 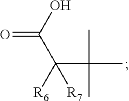
wherein R₆ and R₇, together with the carbon to which they are attached, form a cycloalkyl group, or a pharmaceutically acceptable salt thereof.

23. The compound according to claim 3, wherein R₁ is lower alkyl or indanyl, said lower alkyl being unsubstituted or substituted with cycloalkyl, unsubstituted phenyl or phenyl substituted with halogen or —CF₃; R₂ is hydrogen or lower alkyl; R₃ is hydrogen, fluorine or —OCH₃; X is cycloalkyl acetic acid or 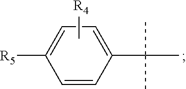
wherein R₄ is hydrogen or halogen and R₅ is methanesulfonylaminocarbonyl-cyclopropyl, or a pharmaceutically acceptable salt thereof.

24. The compound of formula (Ia) according to claim 3, wherein R₁ is lower alkyl being substituted with unsubstituted phenyl; R₂ is lower alkyl; X is 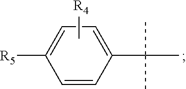
wherein R₄ is hydrogen and R₅ is 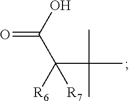
R₆ and R₇ are hydrogen or R₆ and R₇, together with the carbon to which they are attached, form a cycloalkyl group, or a pharmaceutically acceptable salt thereof.

25. The compound of formula (Ib) according to claim 3, wherein R₁ is lower alkyl being substituted with unsubstituted phenyl; R₂ is lower alkyl; R₃ is hydrogen; X is 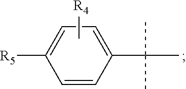
wherein R₄ is hydrogen and R₅ is 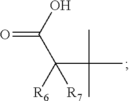
wherein R₆ and R₇ are hydrogen or R₆ and R₇, together with the carbon to which they are attached, form a cycloalkyl group, or a pharmaceutically acceptable salt thereof.

26. The compound of formula (Ic) according to claim 3, wherein R₁ is lower alkyl being substituted with unsubstituted phenyl; R₃ is hydrogen; X is 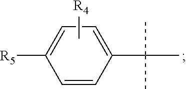
wherein R₄ is hydrogen and R₅ is 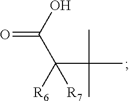
wherein R₆ and R₇, together with the carbon to which they are attached, form a cycloalkyl group, or a pharmaceutically acceptable salt thereof.

27. The compound according to claim 3, wherein said compound is:
1-{4′-[4-Methyl-5-((R)-1-phenyl-ethoxycarbonylamino)-[1,2,3]triazol-1-yl]-biphenyl-4-yl}-cyclopropanecarboxylic acid;
{4′-[4-Methyl-5-((R)-1-phenyl-ethoxycarbonylamino)-[1,2,3]triazol-1-yl]-biphenyl-4-yl}-acetic acid;
1-{4′-[5-Methyl-4-((R)-1-phenyl-ethoxycarbonylamino)-[1,2,3]triazol-1-yl]-biphenyl-4-yl}-cyclopropanecarboxylic acid;
{4′-[5-Methyl-4-((R)-1-phenyl-ethoxycarbonylamino)-[1,2,3]triazol-1-yl]-biphenyl-4-yl}-acetic acid;
1-(4′-{5-[(R)-1-(2-Fluoro-phenyl)-ethoxycarbonylamino]-4-methyl-[1,2,3]triazol-1-yl}-biphenyl-4-yl)-cyclopropanecarboxylic acid;
1-(4′-{4-Methyl-5-[(R)-1-(2-trifluoromethyl-phenyl)-ethoxycarbonylamino]-[1,2,3]triazol-1-yl}-biphenyl-4-yl)-cyclopropanecarboxylic acid;
1-(4′-{4-Methyl-5-[(R)-1-(3-trifluoromethyl-phenyl)-ethoxycarbonylamino]-[1,2,3]triazol-1-yl}-biphenyl-4-yl)-cyclopropanecarboxylic acid;
1-{4′-[5-((R)-Indan-1-yloxycarbonylamino)-4-methyl-[1,2,3]triazol-1-yl]-biphenyl-4-yl}-cyclopropanecarboxylic acid;
1-{4′-[5-((R)-1,2-Dimethyl-propoxycarbonylamino)-4-methyl-[1,2,3]triazol-1-yl]-biphenyl-4-yl}-cyclopropanecarboxylic acid;
1-{4′-[5-((R)-sec-Butoxycarbonylamino)-4-methyl-[1,2,3]triazol-1-yl]-biphenyl-4-yl}-cyclopropanecarboxylic acid;
1-[4′-(5-iso-Propoxycarbonylamino-4-methyl-[1,2,3]triazol-1-yl)-biphenyl-4-yl]-cyclopropanecarboxylic acid;
1-{4′-[5-(1-Cyclopropyl-ethoxycarbonylamino)-4-methyl-[1,2,3]triazol-1-yl]-biphenyl-4-yl}-cyclopropanecarboxylic acid;
1-{4′-[5-(1-Cyclobutyl-ethoxycarbonylamino)-4-methyl-[1,2,3]triazol-1-yl]-biphenyl-4-yl}-cyclopropanecarboxylic acid;
1-[4′-(5-tert-Butoxycarbonylamino-4-methyl-[1,2,3]triazol-1-yl)-biphenyl-4-yl]-cyclopropanecarboxylic acid;
1-{3-Fluoro-4′-[4-methyl-5-((R)-1-phenyl-ethoxycarbonylamino)-[1,2,3]triazol-1-yl]-biphenyl-4-yl}-cyclopropanecarboxylic acid;
1-{3′-Methoxy-4′-[4-methyl-5-((R)-1-phenyl-ethoxycarbonylamino)-[1,2,3]triazol-yl]-biphenyl-4-yl}-cyclopropanecarboxylic acid;
1-{4′-[4-Ethyl-5-((R)-1-phenyl-ethoxycarbonylamino)-[1,2,3]triazol-1-yl]-biphenyl-4-yl}-cyclopropanecarboxylic acid;
{4′-[4-Ethyl-5-((R)-1-phenyl-ethoxycarbonylamino)-[1,2,3]triazol-1-yl]-biphenyl-4-yl}-acetic acid;
1-(4′-{4-Ethyl-5-[(R)-1-(3-trifluoromethyl-phenyl)-ethoxycarbonylamino]-[1,2,3]triazol-1-yl}-biphenyl-4-yl)-cyclopropanecarboxylic acid;
{4′-[4-Ethyl-5-((R)-1-(3-trifluoromethyl-phenyl-ethoxycarbonylamino)[1,2,3]triazol-1-yl]-biphenyl-4-yl}-acetic acid;
1-{4′-[5-((R)-1-Phenyl-ethoxycarbonylamino)-[1,2,3]triazol-1-yl]-biphenyl-4-yl}-cyclopropanecarboxylic acid;
{4′-[5-((R)-1-Phenyl-ethoxycarbonylamino)-[1,2,3]triazol-1-yl]-biphenyl-4-yl}-acetic acid;
2-Methyl-2-{4′-[4-methyl-5-((R)-1-phenyl-ethoxycarbonylamino)-[1,2,3]triazol-1-yl]-biphenyl-4-yl}-propionic acid;
(R)-1-(4′-(4-Methyl-5-((1-phenylethoxy)carbonylamino)-1H-1,2,3-triazol-1-yl)biphenyl-3-yl)cyclopropanecarboxylic acid;
1-{3′-[4-Methyl-5-((R)-1-phenyl-ethoxycarbonylamino)-[1,2,3]triazol-1-yl]-biphenyl-4-yl}-cyclopropanecarboxylic acid;
{3′-[4-Methyl-5-((R)-1-phenyl-ethoxycarbonylamino)-[1,2,3]triazol-1-yl]-biphenyl-4-yl}-acetic acid;
(3-Biphenyl-4-yl-5-methyl-3H-[1,2,3]triazol-4-yl)-carbamic acid (R)-1-phenyl-ethyl ester;
[3-(4′-Cyano-biphenyl-4-yl)-5-methyl-3H-[1,2,3]triazol-4-yl]-carbamic acid (R)-1-phenyl-ethyl ester;
(R)-1-Phenyl-ethyl-1-(4′-(1-(1H-tetrazol-5-yl)cyclopropyl)biphenyl-4-yl)-4-methyl 1H-1,2,3-triazol-5-ylcarbamate;
{3-[4′-(1-Methanesulfonylaminocarbonyl-cyclopropyl)-biphenyl-4-yl]-5-methyl-3H[1,2,3]triazol-4-yl}-carbamic acid (R)-1-phenyl-ethyl ester;
1-{4′-[3-((R)-1-Phenyl-ethoxycarbonylamino)-[1,2,4]triazol-4-yl]-biphenyl-4-yl}-cyclopropanecarboxylic acid;
(R)-1-(4′-(4-Methyl-5-((1-phenylethoxy)carbonylamino)-1H-1,2,3-triazol-1-yl)biphenyl-4-yl)cyclobutanecarboxylic acid;
(R)-2-{4′-[4-Methyl-5-(-1-phenylethoxycarbonylamino)-[1,2,3]triazol-1-yl]-biphenyl-4-yl}-pent-4-enoic acid;
(R)-2-(4-(4-(4-Methyl-5-((1-phenylethoxy)carbonylamino)-1H-1,2,3-triazol-1-yl)phenyl)cyclohexyl)acetic acid; or
{3-[4′-(1-Methanesulfonylaminocarbonyl-cyclopropyl)-biphenyl-4-yl]-5-methyl-3H[1,2,3]triazol-4-yl}-carbamic acid (R)-1-(3-trifluoromethyl-phenyl)-ethyl ester.

28. (canceled)

29. A pharmaceutical composition, comprising a therapeutically effective amount of a compound in according to claim 3 and a therapeutically inert carrier.

30-32. (canceled)

33. A method for the treatment or prophylaxis of pulmonary fibrosis, which method comprises the step of administering an effective amount of a compound according to claim 3 to a patient in need thereof.

34. (canceled)
